(19) United States
(12) Patent Application Publication
(10) Pub. No.: US 2014/0358198 A1
(43) Pub. Date: Dec. 4, 2014

(54) ORGANIC SENSITIZERS FOR UP-CONVERSION

(71) Applicant: Merck Patent GmbH, Darmstadt (DE)

(72) Inventors: Herwig Buchholz, Frankfurt am Main (DE); Junyou Pan, Frankfurt am Main (DE); Susanne Heun, Bad Soden (DE)

(21) Appl. No.: 14/363,969

(22) PCT Filed: Dec. 5, 2012
(86) PCT No.: PCT/EP2012/005017

§ 371 Date: Jun. 9, 2014

(30) Foreign Application Priority Data
Dec. 13, 2011 (DE) 10 2011 121 022.2

(51) Int. Cl. A61N 5/06; H01L 51/50; C09K 11/06

(52) U.S. Cl. CPC A61N 5/0616; C09K 11/06; H01L 51/5036; C09K 2211/1011; A61N 2005/0663; A61N 2005/0653; A61N 2005/0661
USPC 607/88; 252/301.35; 252/301.16

(57) ABSTRACT

The invention relates to compositions, devices containing same, and the use thereof for up-conversion. Said compositions and devices are particularly suitable for generating low-energy radiation.

The present invention relates to the provision of novel compositions comprising sensitisers for up-conversion by means of TTA (triplet-triplet annihilation), and to electronic devices comprising these compositions.

Up-conversion (UpC) is very generally taken to mean the generation of high-energy excitons from low-energy excitons, where the low-energy excitons are generated either by electrical, electromagnetic or optical excitation and the energy of the high-energy excitons is released again at least partly in the form of photons. UpC has already been observed in a number of organic materials (T. Kojei et al., Chem. Phys. Lett. 1998, 298, 1; G. S. He et al., Appl. Phys. Lett. 1996, 68, 3549; R. Schroeder et al., J. Chem. Phys. 2002, 116, 3449; J. M. Lupton, Appl. Phys. Lett. 2002, 80, 186; C. Bauer et al., Adv. Mater. 2002, 14, 673).

In order to achieve UpC, various processes are employed, for example the simultaneous absorption of two or more low-energy photons using coherent light sources (lasers). However, very high light intensities in the range from MW/cm² to GW/cm² are required for this purpose, since the two low-energy photons have to be absorbed virtually simultaneously. This is a non-linear optical effect, which generally proceeds with relatively low conversion efficiency. Another process proposes sequential multiphoton absorption. Here too, very high intensities are required, since states which are already excited have to be excited further with the photons arriving later. Since the use of these mechanisms is associated with considerable effort and nevertheless generally only results in low energy densities of doubled radiation, they represent a hindrance to practical use. However, this would be very desirable, since often only the higher-energy part of the spectrum (blue) is effective for applications, but the stability of high-energy blue states is significantly lower than that of low-energy red states.

UpC has been described, for example, in a system comprising a methyl-substituted conductor polymer (MeLPPP) doped with platinum octaethylporphyrin (PtOEP) as sensitiser (S. A. Bagnich, H. Bässler, Chem. Phys. Lett. 2003, 381, 464) and on polyfluorene, doped with metal(II) octaethylporphyrin (P. E. Keivanidis et al., Adv. Mater. 2003, 15, 2095). The presence of the metal complex enabled the pump intensities of the laser to be reduced by five orders of magnitude compared with up-conversion on simple polyfluorene systems containing no metal complexes. However, the efficiency of the up-conversion is low, the ratio of the integrated photoluminescence of the polyfluorene on excitation of the metal complex compared with direct excitation of the polyfluorene has been determined as 1:5000 for palladium porphyrin. The use of platinum porphyrin enabled the efficiency to be improved by a factor of 18, giving approximately a ratio of 1:300. However, the emission of the metal complex remains clearly perceptible in these systems and is thus an interfering loss channel.

If it were possible to use an efficient process for UpC, this would have far-reaching consequences for numerous applications. Thus, a possible application is in the area of organic solar cells (O-SC). Hitherto, usually only visible (typically blue and green) and UV components of the incident light contribute to the generation of free charge carriers here, since the bonding energy of hole and electron has to be overcome in this process. With the aid of UpC, however, red or even infrared light can also contribute to the device efficiency if higher-energy blue or green light can be generated therefrom, which can then be absorbed again or can generate high-energy excitons via Förster transfer. It may even be possible to generate free charge carriers directly.

A further application is in the area of organic light-emitting diodes, for example for the generation of blue or white light by simultaneous emission of blue and red/yellow. Existing applications which would profit from more efficient UpC are, for example, in the area of organic blue lasers, which can be pumped by means of commercially available green or red lasers. Thus, scattering and linear absorption can be reduced and the photostability of the material can be increased. A further possible application is, for example, in crosslinking reactions, where the use of green light enables the generation of UV light, whose action on a sensitiser is able to initiate the crosslinking reaction. Still a further possible application are switches in the case of which only a certain wavelength at which a relatively narrow-band sensitiser absorbs triggers the emission of blue light. Also possible are systems for optical data storage, biological or medical applications, etc.

A further highly promising process for achieving UpC is triplet-triplet annihilation (TTA; Figure 1; Cheng et al., Phys. Chem. Chem. Phys., 12, 66 (2010); Baluschev et al. Appl. Phys. Lett. 90, 181103 (2007); J. E. Auckett et al. J. Phys: Conference Series 185 (2009) 012002). A sensitiser (I) is excited from the ground state S₀ into an excited singlet state (S₁) by means of the energy Eᵢₙ. Intersystem crossing (ISC), i.e. the transition into the first excited triplet state T₁ with spin inversion, occurs. Energy transfer then takes place from T₁ of the sensitiser to the T₁ level of the acceptor (II) (TTET—triplet-triplet energy transfer), with phosphorescence hν₁ from T₁ of the sensitiser being possible as a competing process. Finally, a bimolecular impact between two acceptors, both of which are in the excited T₁ state, results in the first acceptor being converted into the excited Sₙ state and the other acceptor being converted into the electronic ground state S₀ (T₁+T₁→Sₙ+S₀). After relaxation (IC—internal conversion) from Sₙ to S₁, the emission hνₒᵤₜ of the acceptor occurs from the S₁ state. A crucial advantage of TTA up-conversion (TTA-UpC) is that it is independent of the formation history and, so long as the molecules are located sufficiently close to one another, the occupation density of the states.

The sensitisers employed for TTA-UpC are also usually organic metal complexes, since the presence of a heavy atom considerably increases the intersystem crossing rate (ISC) owing to spin-orbit coupling. Owing to this strong spin-orbit coupling, however, the emitting conversion probability from T₁ to Sₒ (phosphorescence) is also increased, which results in a reduction in the efficiency of TTA-UpC and an additional, non-up-converted emission. To date, only few organic sensitisers which contain no heavy atoms are known for TTA-UpC. In a recently published paper by J. Zhao et al. (RSC Advances, 1, 937 (2011)), organic sensitisers which utilise an iodine substitution instead of the typical metal ion have been published for use in photoluminescence. These are molecules based on BODIPY, which on the one hand utilise the known high absorption of this class of fluorescence dyes, but, owing to the increased ISC rate, suppress the fluorescence quantum yield. However, the substitution by iodine is undesired for use in organic electroluminescent devices. Furthermore, the authors disclose few further organic sensitisers (2,3-butanedione, acridone and diphenyl ketone) which are suitable for UpC in the context of photoluminescence, but are unsuitable for electroluminescent applications owing to their low boiling points or electronic instability.

Owing to the relatively low UpC efficiency of the systems to date, UpC processes have, irrespective of the underlying physical principle, hitherto played no role in optoelectronics. In order to utilise red light in organic solar cells, a significant proportion of blue light would have to be formed therein in order thus additionally to generate free charge carriers. In OLEDs, triplet states form in large number owing to the spin statistics, but these are generally non-luminescent and thus represent a loss channel (exception are the above-mentioned components with organometallic heavy-atom complexes in the emission layer). With the aid of efficient UpC, these nonemitting states could be converted into emitting higher-energy singlet states and thus nevertheless contribute to the device efficiency.

It would therefore be desirable for the application to achieve a significant further increase in the TTA-UpC and further to increase the efficiency of the existing systems and sensitisers. This was the object on which the invention was based.

It has now been found, surprisingly, that certain organic sensitisers without heavy atoms are particularly suitable for TTA-UpC, exhibit very good efficiencies and have no interfering residual emission. These are suitable both as sensitisers for photoluminescence and also for electroluminescence.

The present invention therefore relates to a composition for up-conversion, preferably for up-conversion in electroluminescent devices, comprising at least one sensitiser, which is a polymer, oligomer, dendrimer or small molecule, and at least one fluorescent organic emitter, characterised in that the sensitiser contains structural units selected from the following compounds having the general formulae (1) and (2), with the proviso that the emitter is not an organic metal complex

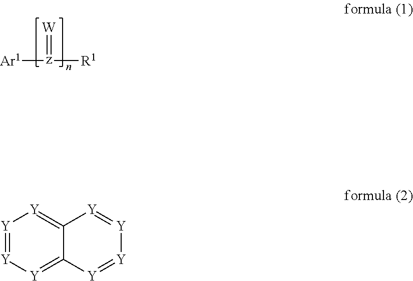

where the following applies to the symbols used:
n is either 1, 2 or 3, preferably 1 or 2 and very preferably 1;
W is, identically or differently on each occurrence, equal to O, S or Se, preferably O or S, very preferably O;
Ar¹ an aromatic or heteroaromatic ring or an aromatic or heteroaromatic ring system, where the rings may be substituted by one or more radicals R¹, with the proviso that Ar¹ contains at least 9 ring atoms;
Y is, identically or differently on each occurrence, equal to N or C(R¹), where at least 2 of the Y are equal to N, preferably at least two of the Y are equal to N and at least one Y is equal to C(R¹), where R¹ is not equal to H;
Z is, identically or differently on each occurrence, C, S or S(═O), preferably C;
R¹ is, identically or differently on each occurrence, H, D, F, Cl, Br, I, N(R²)₂, CN, Si(R²)₃, B(OR²)₂, C(═O)R², P(═O)(R²)₂, S(═O)R², S(═O)₂R², OSO₂R², a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which may be substituted by one or more radicals R², where one or more non-adjacent CH₂ groups may be replaced by R²C═CR², C═C, Si(R²)₂, Ge(R²)₂, Sn(R²)₂, C═O, C═S, C═Se, C═NR², P(═O)(R²), SO, SO₂, NR², O, S or CONR² and where one or more H atoms may be replaced by D, F, Cl, Br, I, CN or NO₂, or an aromatic or heteroaromatic ring system having 5 to 60 aromatic ring atoms, which may in each case be substituted by one or more radicals R², or an aryloxy, arylalkoxy or heteroaryloxy group having 5 to 60 aromatic ring atoms, which may be substituted by one or more radicals R², or a diarylamino group, diheteroarylamino group or arylheteroarylamino group having 10 to 40 aromatic ring atoms, which may be substituted by one or more radicals R², or a combination of two or more of these groups or a crosslinkable group Q;
R² is, identically or differently on each occurrence, H, D, F, Cl, Br, I, N(R³)₂, CN, NO₂, Si(R³)₃, B(OR³)₂, C(═O)R³, P(═O)(R³)₂, S(═O)R³, S(═O)₂R³, OSO₂R³, a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which may be substituted by one or more radicals R³, where one or more non-adjacent CH₂ groups may be replaced by R³C═CR³, C═C, Si(R³)₂, Ge(R³)₂, Sn(R³)₂, C═O, C═S, C═Se, C═NR³, P(═O)(R³), SO, SO₂, NR³, O, S or CONR³ and where one or more H atoms may be replaced by D, F, Cl, Br, I, CN or NO₂, or an aromatic or heteroaromatic ring system having 5 to 60 aromatic ring atoms, which may in each case be substituted by one or more radicals R³, or an aryloxy, arylalkoxy or heteroaryloxy group having 5 to 60 aromatic ring atoms, which may be substituted by one or more radicals R³, or a diarylamino group, diheteroarylamino group or arylheteroarylamino group having 10 to 40 aromatic ring atoms, which may be substituted by one or more radicals R³, or a combination of two or more of these groups; two or more adjacent radicals R² here may form a mono- or polycyclic, aliphatic or aromatic ring system with one another;
R³ is, identically or differently on each occurrence, H, D, F or an aliphatic, aromatic and/or heteroaromatic hydrocarbon radical having 1 to 20 C atoms, in which, in addition, one or more H atoms may be replaced by F; two or more substituents R³ here may also form a mono- or polycyclic, aliphatic or aromatic ring system with one another.

“Crosslinkable group” in the sense of the present invention denotes a functional group which is capable of reacting irreversibly. A crosslinked material, which is insoluble, is thereby formed. The crosslinking can usually be supported by heat or by UV, microwave, X-ray or electron radiation. Examples of crosslinkable groups Q are units which contain a double bond, a triple bond, a precursor which is capable of forming a double or triple bond in situ, or a heterocyclic addition-polymerisable radical. Preferred radicals Q include vinyl, alkenyl, preferably ethenyl and propenyl, C₄₋₂₀-cycloalkenyl, azide, oxirane, oxetane, di(hydrocarbyl)amino, cyanate ester, hydroxyl, glycidyl ether, C₁₋₁₀-alkyl acrylate, C₁₋₁₀-alkyl methacrylate, alkenyloxy, preferably ethenyloxy, perfluoroalkenyloxy, preferably perfluoroethenyloxy, alkynyl, preferably ethynyl, maleimide, tri(C₁₋₄)-alkylsiloxy and tri(C₁₋₄)-alkylsilyl. Particular preference is given to vinyl and alkenyl.

A small molecule in the sense of the present invention is a molecule which is not a polymer, oligomer or dendrimer or a mixture (blend) thereof. In particular, small molecules differ from polymers, oligomers or dendrimers through the fact that they contain no recurring units. The molecular weight of small molecules is typically in the region of polymers and oligomers having few recurring units and below. The molecular weight of small molecules is preferably less than 4000 g/mol, very preferably less than 3000 g/mol and very particularly less than 2000 g/mol.

Polymers have 10 to 10000, preferably 20 to 5000 and very preferably 50 to 2000 recurring units. Oligomers have 2 to 9 recurring units. The branching index of polymers and oligomers is between 0 (linear polymer with no branching) and 1 (fully branched polymer). The term dendrimer herein is understood as described by M. Fischer et al. in Angew. Chem., Int. Ed. 1999, 38, 885.

The molecular weight (M_{W}) of polymers is preferably in the range between about 10000 and about 2000000 g/mol, very preferably between about 100000 and about 1500000 g/mol, and very particularly preferably between about 200000 and about 1000000 g/mol. M_{W} is determined by methods which are well known to the person skilled in the art, by means of gel permeation chromatography (GPC) with polystyrene as internal standard, for example.

A mixture (blend) is taken to mean a mixture which comprises at least one polymeric, dendritic or oligomeric component.

The triplet energy of a compound is taken to mean the energy difference between the lowest triplet state T₁ and the singlet ground state S₀.

The energy difference between T₁ and S₀, simply called triplet level T₁ hereinbelow, can be calculated both by means of spectroscopy and also by means of quantum-chemical simulation (time-dependent DFT). For organic compounds which contain no metal, T₁ can be measured by time-resolved spectroscopy at low temperatures as follows: 100 nm of a film produced, for example, by spin coating or of an amorphous vapour-deposited layer on quartz are excited by a tripled YAG laser (@ 355 nm) or an N₂ laser (@ 337 nm) at helium temperature (<10 K). The delayed photoluminescence is recorded by so-called “gated” detection after a certain time (for example 1 μs). The wavelength of the emission in the time window of delayed luminescence then corresponds to the transition from T₁ to S₀ and thus, converted into energy values, to the T₁ level of the system investigated. The simulation method for T₁ is described in greater detail in the examples. The correlation between measurement and simulation is known to be very good.

A sensitiser in the sense of this invention is taken to mean a compound which absorbs light in the region of the incident wavelength and is then converted into a triplet state by intersystem crossing (ISC), or which generates triplet states on electrical excitation through recombination of electrons and holes and can optionally subsequently transfer these to the acceptor molecule by triplet-triplet energy transfer (TTET). The triplet level of the sensitiser here is above the triplet level of the acceptor molecule.

Phosphorescence in the sense of the present application is taken to mean luminescence from an excited state having relatively high spin multiplicity, i.e. from a state having a spin quantum number S greater than or equal to 1. Phosphorescence in the sense of the present invention is preferably taken to mean luminescence in the case of which radiation is emitted from an excited triplet (S=1, 2S+1=3) and/or from an excited quintet (S=2, 2S+1=5) state, very preferably from an excited triplet state.

Fluorescence in the sense of the present invention is taken to mean luminescence from an excited singlet state, preferably from the first excited singlet state S₁.

An aryl group in the sense of this invention contains 6 to 40 C atoms; a heteroaryl group in the sense of this invention contains 1 to 39 C atoms and at least one heteroatom, with the proviso that the sum of C atoms and heteroatoms is at least 5. The heteroatoms are preferably selected from N, O and/or S. An aryl group or heteroaryl group here is taken to mean either a simple aromatic ring, i.e. benzene, or a simple heteroaromatic ring, for example pyridine, pyrimidine, thiophene, etc., or a condensed (anellated) aryl or heteroaryl group, for example naphthalene, anthracene, phenanthrene, quinoline, isoquinoline, etc. Aromatic groups which are linked to one another by a single bond, such as, for example, biphenyl, are, by contrast, not referred to as aryl or heteroaryl group, but instead as aromatic ring system.

An aromatic ring system in the sense of this invention contains 6 to 60 C atoms in the ring system. A heteroaromatic ring system in the sense of this invention contains 1 to 59 C atoms and at least one heteroatom in the ring system, with the proviso that the sum of C atoms and heteroatoms is at least 5. The heteroatoms are preferably selected from N, O and/or S. For the purposes of this invention, an aromatic or heteroaromatic ring system is intended to be taken to mean a system which does not necessarily contain only aryl or heteroaryl groups, but instead in which, in addition, a plurality of aryl or heteroaryl groups may be linked by a non-aromatic unit, such as, for example, a C, N or O atom. Thus, for example, systems such as fluorene, 9,9′-spirobifluorene, 9,9-diarylfluorene, triarylamine, diaryl ether, stilbene, etc., are also intended to be taken to be aromatic ring systems for the purposes of this invention, as are systems in which two or more aryl groups are interrupted, for example, by a short alkyl group. Furthermore, systems in which a plurality of aryl and/or heteroaryl groups are linked to one another by a single bond, such as, for example, biphenyl, terphenyl or bipyridine, are intended to be taken to be an aromatic or heteroaromatic ring system.

For the purposes of the present invention, an aliphatic hydrocarbon radical or an alkyl group or an alkenyl or alkynyl group, which may typically contain 1 to 40 or also 1 to 20 C atoms and in which, in addition, individual H atoms or CH₂ groups may be substituted by the above-mentioned groups, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl. An alkoxy group having 1 to 40 C atoms is preferably taken to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 C atoms is taken to mean, in particular, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups in accordance with the present invention may be straight-chain, branched or cyclic, where one or more non-adjacent CH₂ groups may be replaced by R¹C═CR¹, C═C, Si(R¹)₂, Ge(R¹)₂, Sn(R¹)₂, C═O, C═S, C═Se, C═NR¹, P(═O)(R¹), SO, SO₂, NR¹, O, S or CONR¹; furthermore, one or more H atoms may also be replaced by D, F, Cl, Br, I, CN or NO₂, preferably F, CI or CN, further preferably F or CN, particularly preferably CN.

An aromatic or heteroaromatic ring system having 5-60 aromatic ring atoms, which may also in each case be substituted by the above-mentioned radicals R¹ or a hydrocarbon radical and which may be linked to the aromatic or heteroaromatic ring system via any desired positions, is taken to mean, in particular, groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The fact that the sensitiser contains structural units selected from the following compounds having the general formulae (1) and (2) means that the sensitiser either corresponds precisely to the compounds of the formulae (1) and/or (2) or contains the structures of the formulae (1) and/or (2) as sub-structure. Thus, these may also be polymers, oligomers or dendrimers into which structures of the formulae (1) and/or (2) have been incorporated.

The substituents Ar¹ and R¹ in the compound of the formula (1) may also be connected to one another by covalent bonds in order to form, for example, a cyclic or polycyclic ring system.

It is preferred for the purposes of the present invention for the sensitiser to be a small molecule having the structure of the general formula (1) or (2).

In a preferred embodiment of the present invention, the sensitiser of the formula (1) is selected from the compounds of the formulae (3) to (7), very preferably from the compounds of the formulae (3), (4) and (5), very particularly preferably from the compounds of the formulae (3) and (5) and especially preferably from the compounds of the formula (3).

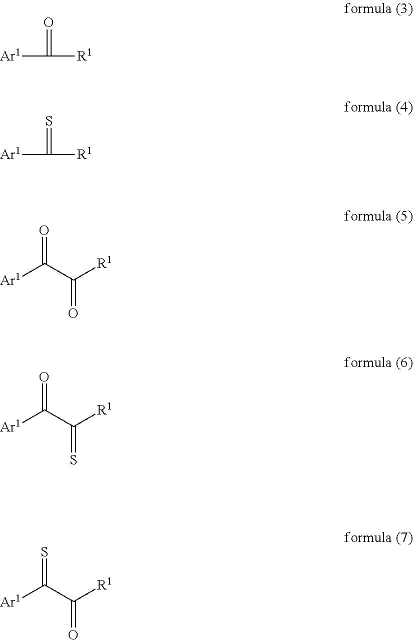

where the radicals Ar¹ and R¹ are defined as indicated above.

Preferred groups for Ar¹ are selected from the group of the aromatic or heteroaromatic rings or ring systems, for example from the group of the fluorenes, spriobifluorenes, phenanthrenes, indenofluorenes, carbazolene, indenocarbazolene, indolocarbazoles, dihydrophenanthrenes, naphtalenene, antharcenes, pyrenes, triazines and benzanthracenes, triarylamines, dibenzofuran, azaboroles, diazasiloles, diazaphospholes, azacarbazoles, benzidines, tetraaryl-para-phenylenediamines, triarylphosphines, phenothiazines, phenoxazines, dihydrophenazines, thianthrenes, dibenzoparadioxins, phenoxathiynes, azulenes, perylenylenes, biphenylylenes, terphenylylenes, tolanylenes, stilbenylenes, bisstyrylarylenes, benzothiadiazoles, quinoxalines, phenothiazines, phenoxazines, dihydrophenazines, bis(thiophenyl)arylenes, oligo(thiophenylene)s, phenazines, rubrenes, pentacenes, perylenes and of derivatives thereof.

Examples thereof are 4,5-dihydropyrenes, 4,5,9,10-tetrahydropyrenes and fluorenes as disclosed in U.S. Pat. No. 5,962,631, WO 2006/052457 A2 and in WO 2006/118345A1, 9,9′-spirobifluorenes, as disclosed in WO 2003/020790 A1, 9,10-phenanthrenes, as disclosed in WO 2005/104264 A1, 9,10-dihydrophenanthrenes, as disclosed in WO 2005/014689 A2, 5,7-dihydrodibenzoxepines and cis- and transindenofluorenes, as disclosed in WO 2004041901 A1 and WO 2004113412 A2, binaphthylenes, as disclosed in WO 2006/063852 A1 and further units, as disclosed in WO 2005/056633A1, EP 1344788A1, WO 2007/043495A1, WO 2005/033174 A1, WO 2003/099901 A1 and DE 102006003710.

Ar¹ in the formulae (1) to (7) is preferably selected from the groups of the following formulae (8) to (14)

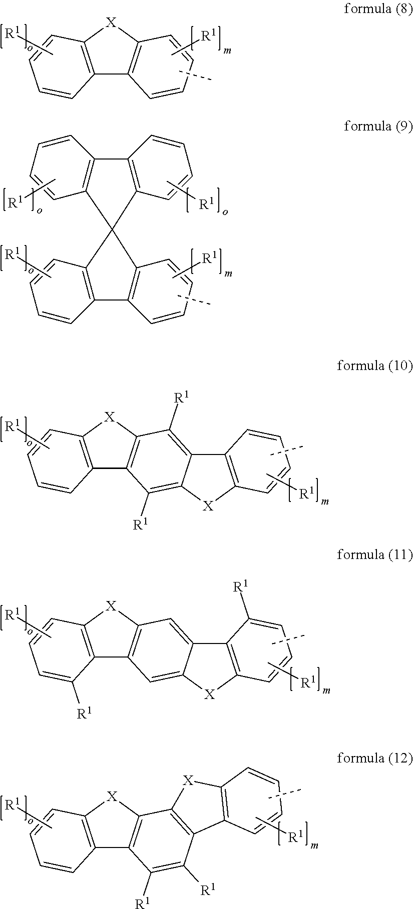

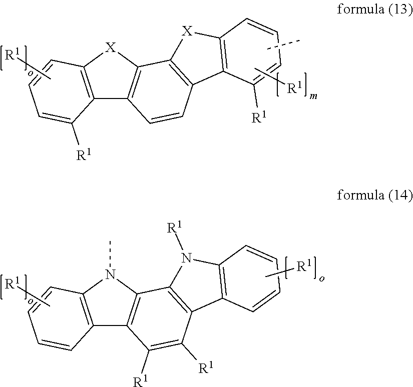

where R¹ has the same meaning as described above, the dashed bond represents the linking position, and furthermore:
X is, identically or differently on each occurrence, a divalent bridge selected from B(R¹), C(R¹)₂, Si(R¹)₂, C═O, C═NR¹, C═C(R¹)₂, O, S, S═O, SO₂, N(R¹), P(R¹) and P(═O)R¹;
m is on each occurrence, identically or differently, 0, 1, 2 or 3;
o is on each occurrence, identically or differently, 0, 1, 2, 3 or 4.

Particularly preferred groups Ar¹ are selected from the groups of the following formulae (15) to (23),

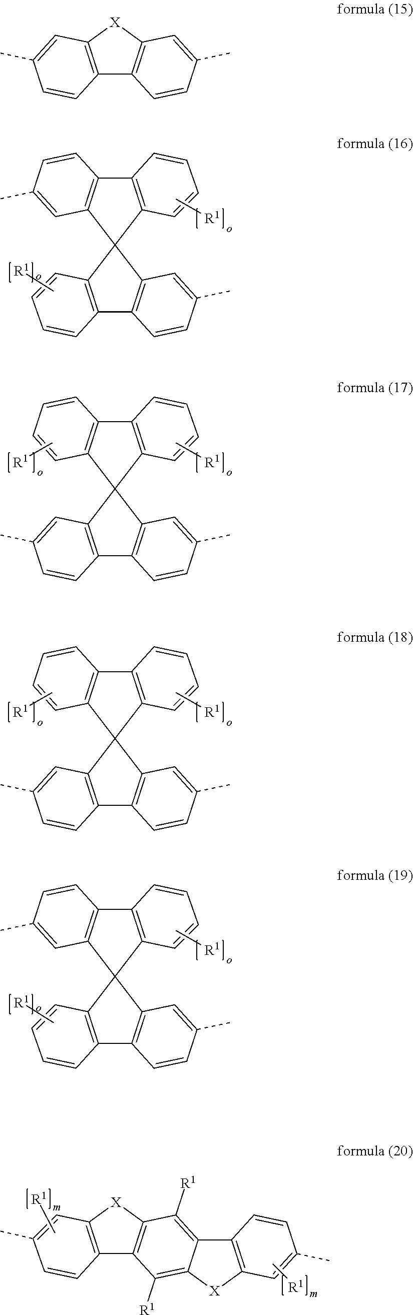

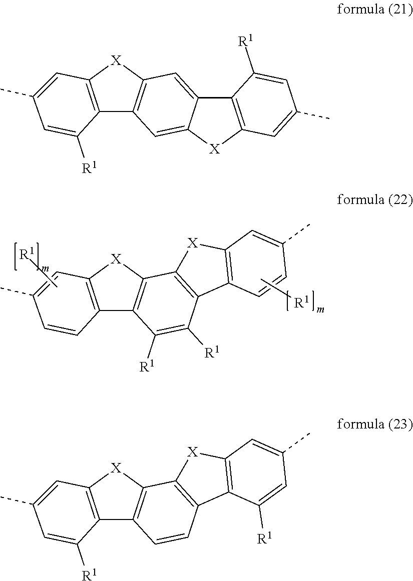

where the symbols and indices used have the same meaning as described above. X here is preferably selected, identically or differently, from C(R¹)₂, N(R¹), O and S, particularly preferably C(R¹)₂.

In a furthermore preferred embodiment of the present invention, R¹ in the compounds of the formulae (8) to (23) is equal to Ar¹. Furthermore preferably, X is selected, identically or differently, from C(R²)₂, N(R), O and S, particularly preferably C(R²)₂, where R² is defined as in formula (1) and (2).

In a furthermore preferred embodiment of the present invention, the sensitiser of the formula (2) is selected from the compounds of the general formula (24).

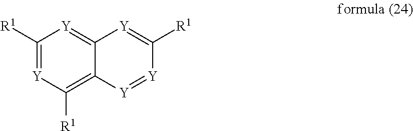

where Y and R¹ have the meanings indicated above.

The sensitiser furthermore very preferably has the structure of the following formulae (25) to (27), very particularly preferably formula (25).

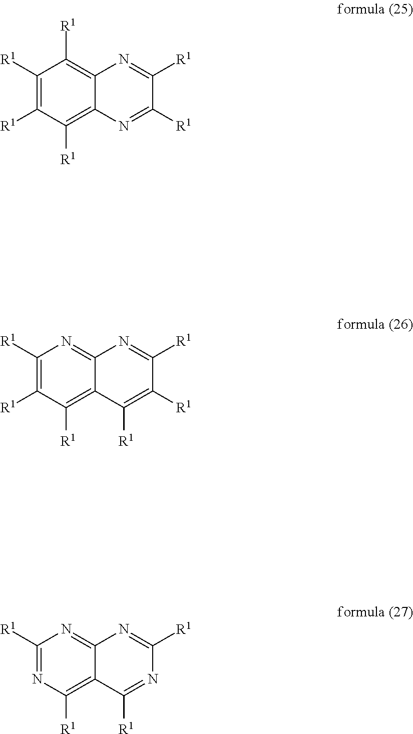

The sensitiser very particularly preferably has the structure of the following formulae (28) to (33), especial preference is given to the compounds of the formulae (28), (29) and (30), and even more preference is given to those of the formulae (28) and (29).

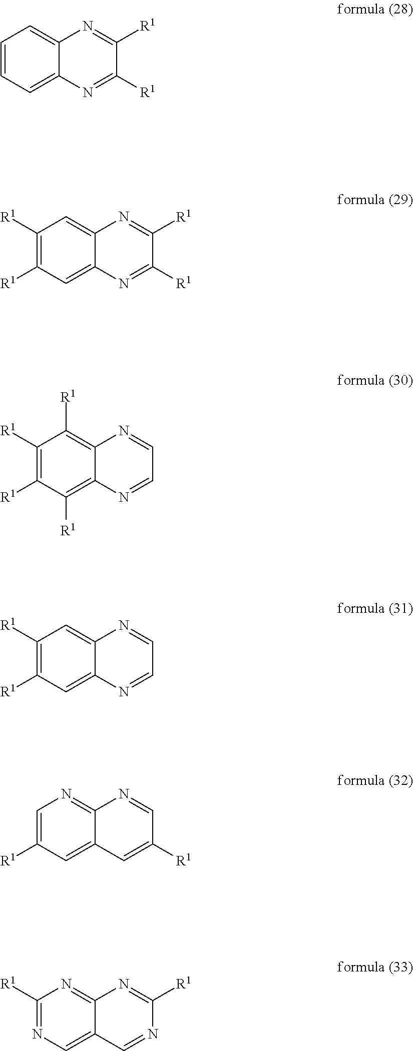

R¹ in the compounds of the formulae (1) to (33) is preferably, identically or differently on each occurrence, N(R²)₂, CN, Si(R²)₃, a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which may be substituted by one or more radicals R², where one or more non-adjacent CH₂ groups may be replaced by R²C═CR², C═C, Si(R²)₂, Ge(R²)₂, Sn(R²)₂, C═O, C═S, C═Se, C═NR², P(═O)(R²), SO, SO₂, NR², O, S or CONR² and where one or more H atoms may be replaced by D, F, Cl, Br, I, CN or NO₂, or an aromatic or heteroaromatic ring system having 5 to 60 aromatic ring atoms, which may in each case be substituted by one or more radicals R², or an aryloxy, arylalkoxy or heteroaryloxy group having 5 to 60 aromatic ring atoms, which may be substituted by one or more radicals R², or a diarylamino group, diheteroarylamino group or arylheteroarylamino group having 10 to 40 aromatic ring atoms, which may be substituted by one or more radicals R², or a combination of two or more of these groups or a crosslinkable group Q;

R¹ in the formulae (3) to (7) and (24) to (33) is very preferably selected, identically or differently on each occurrence, from formula (8) to (23) and one of the following formulae (34) to (222), where the compounds having the formulae (34) to (222) indicated may be substituted by one or more, identical or different radicals R², where R² has been defined above.

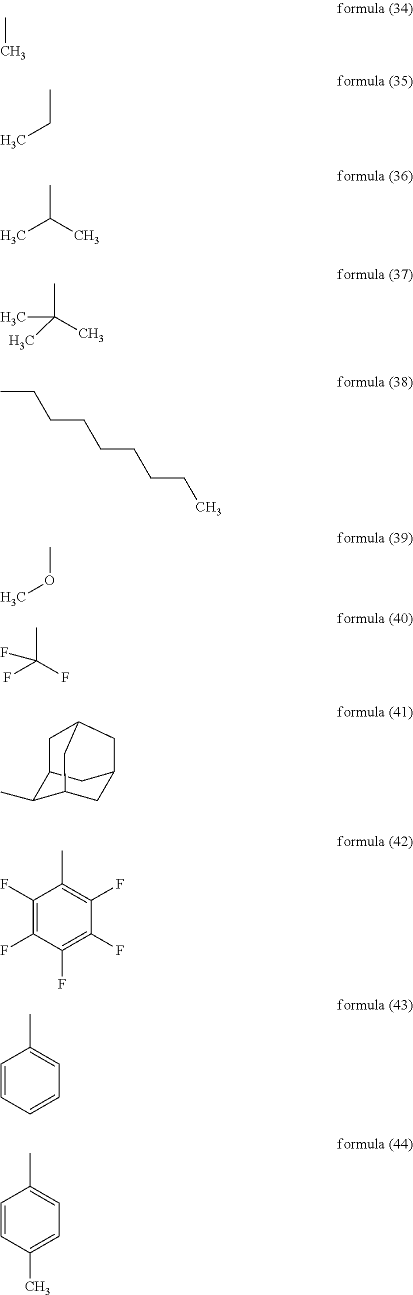

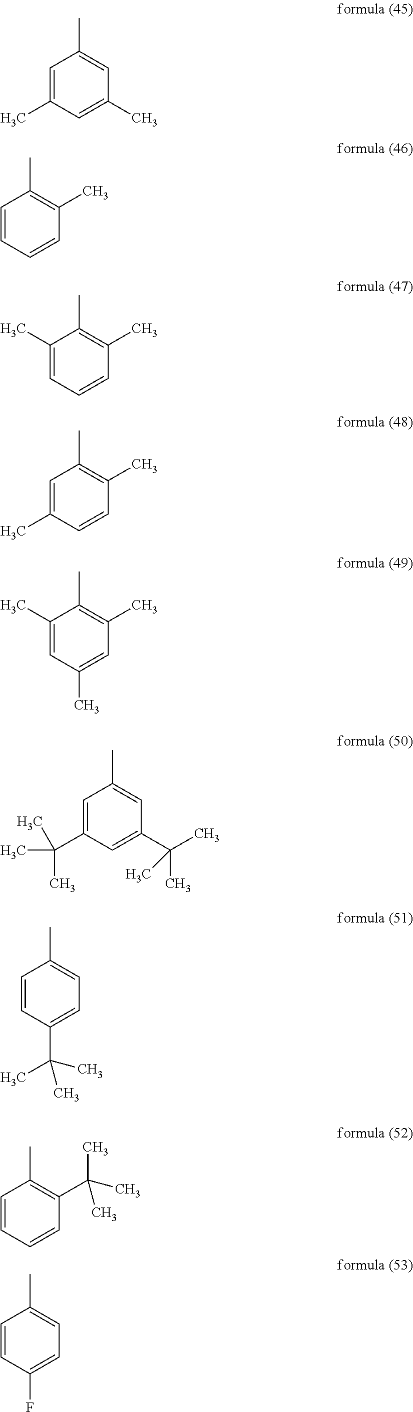

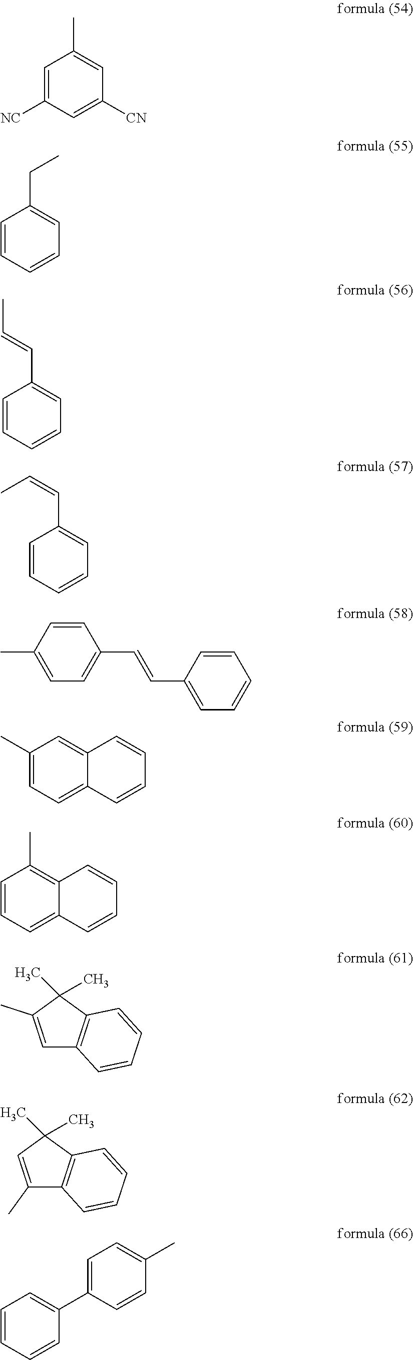

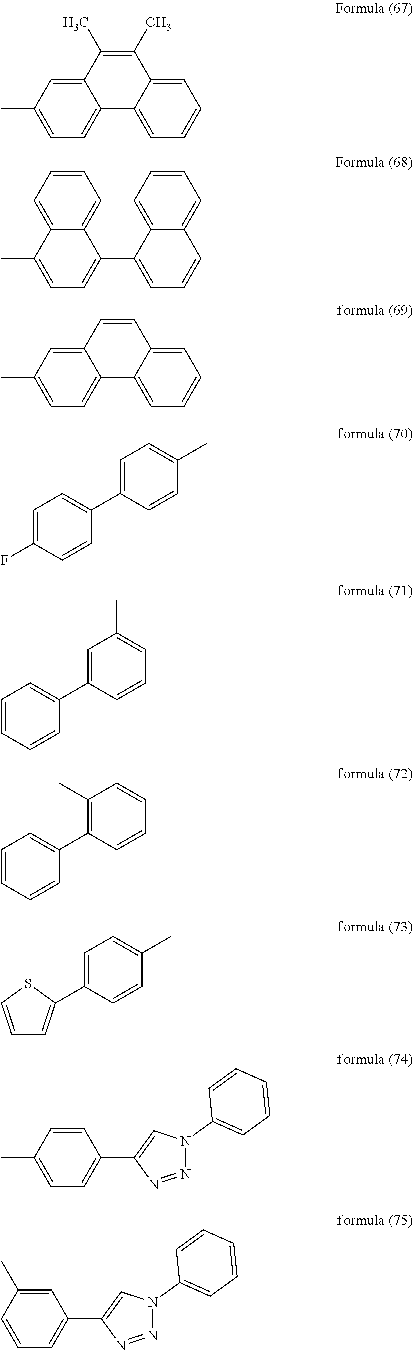

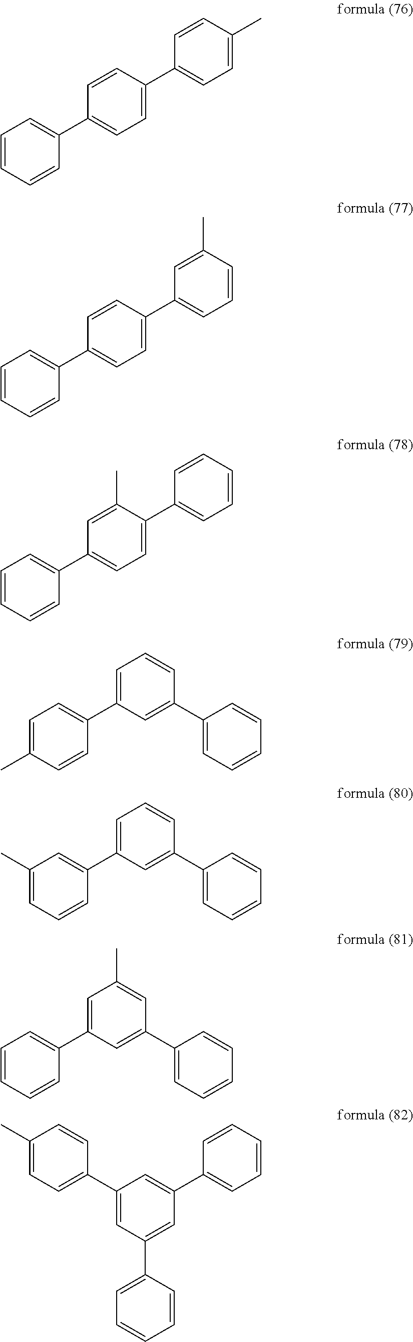

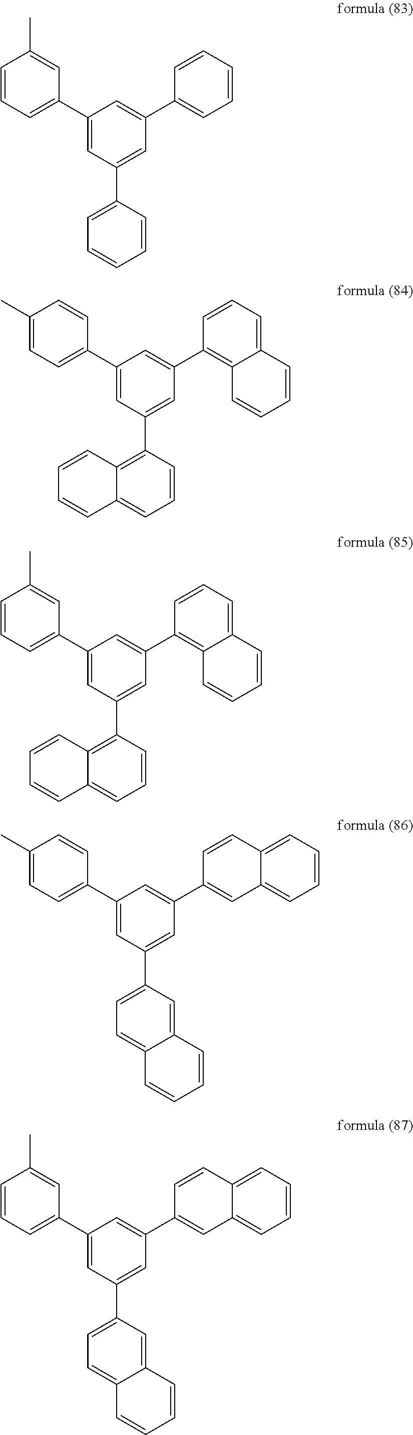

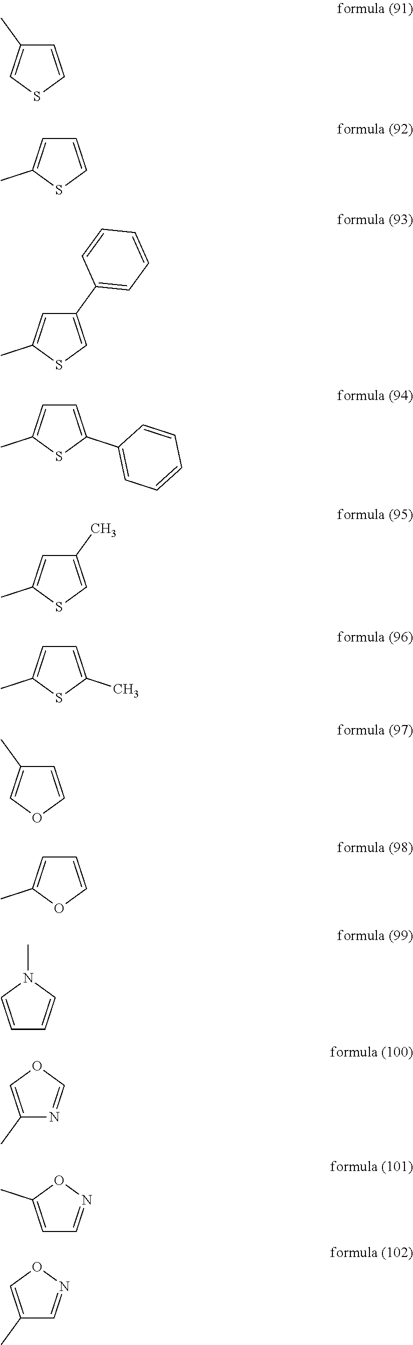

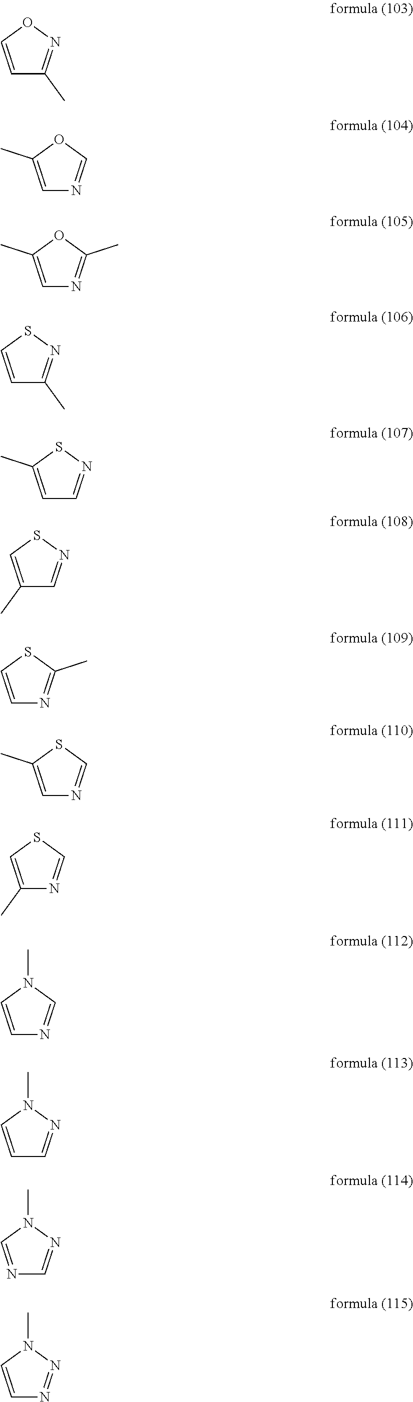

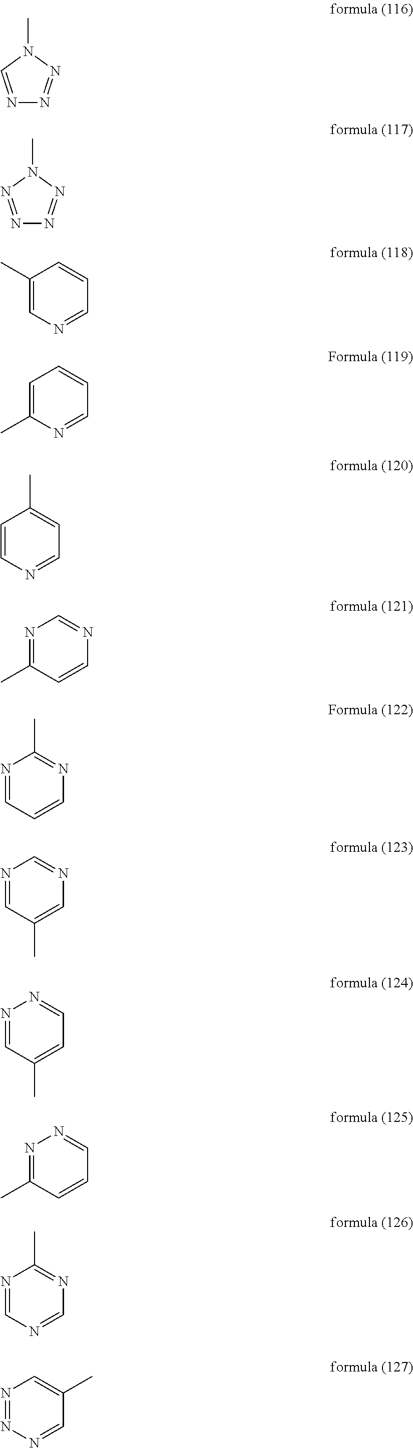

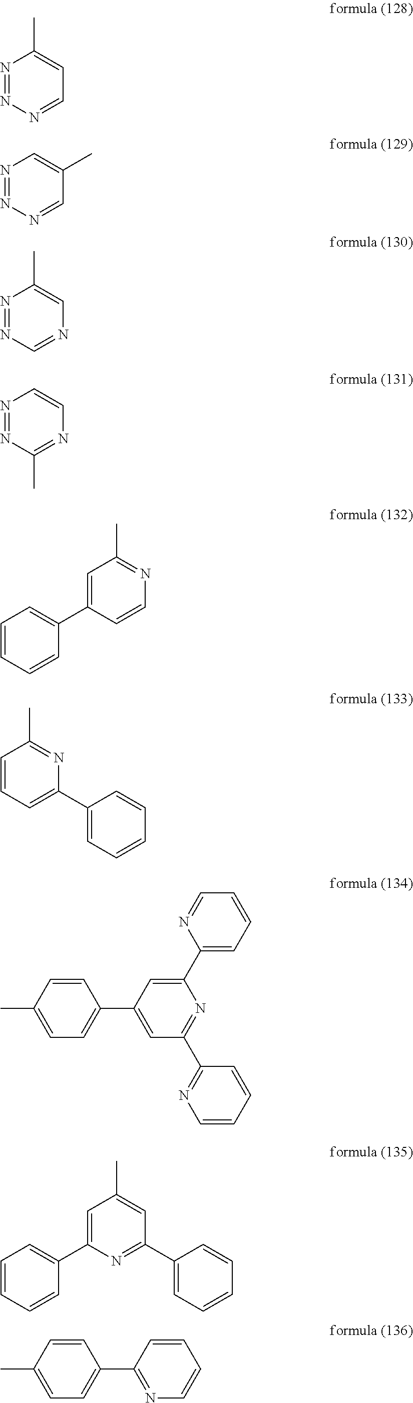

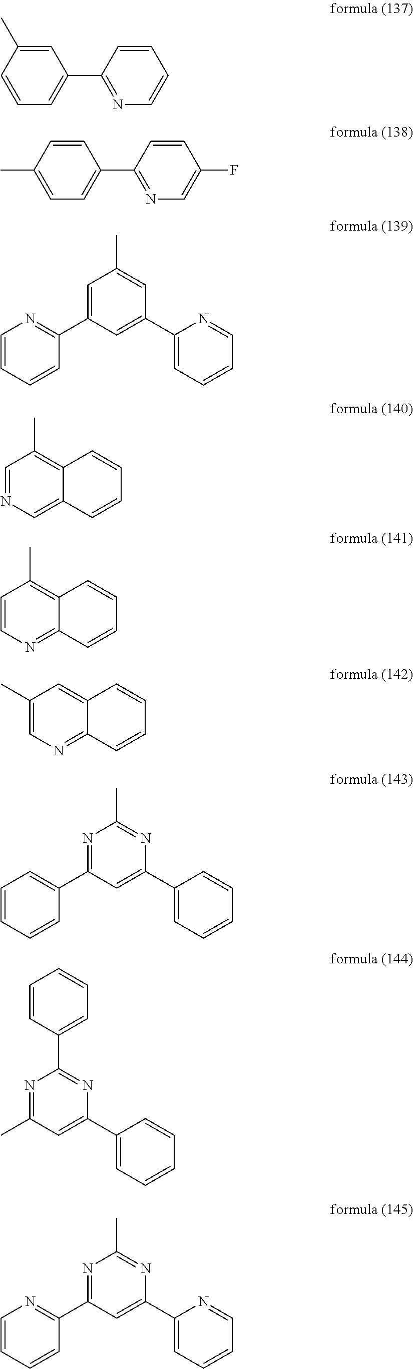

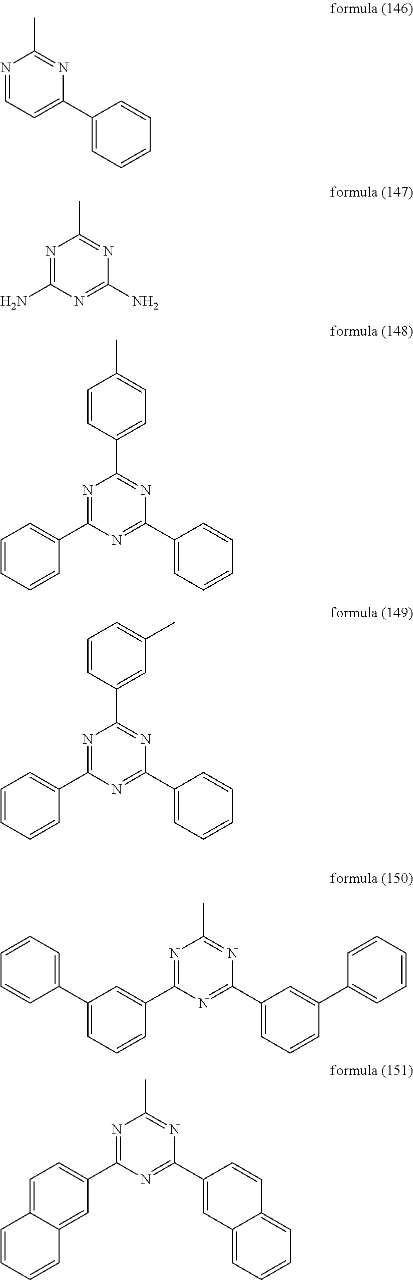

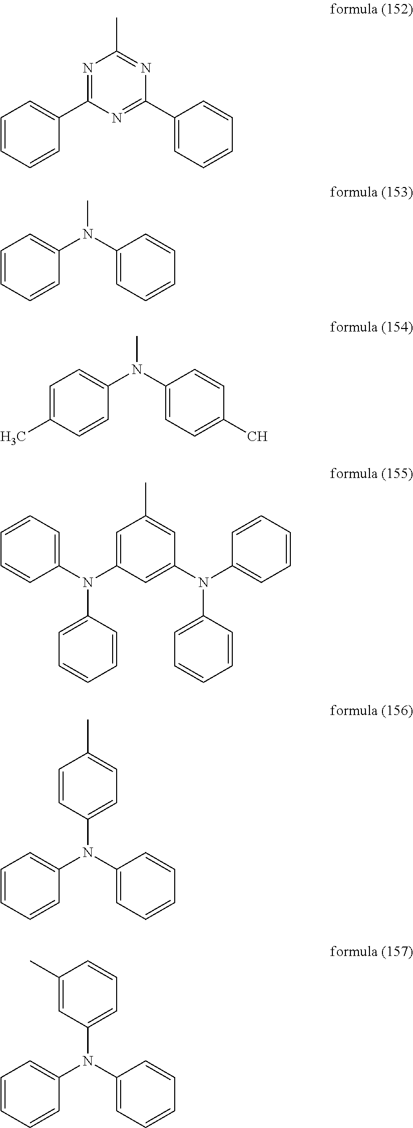

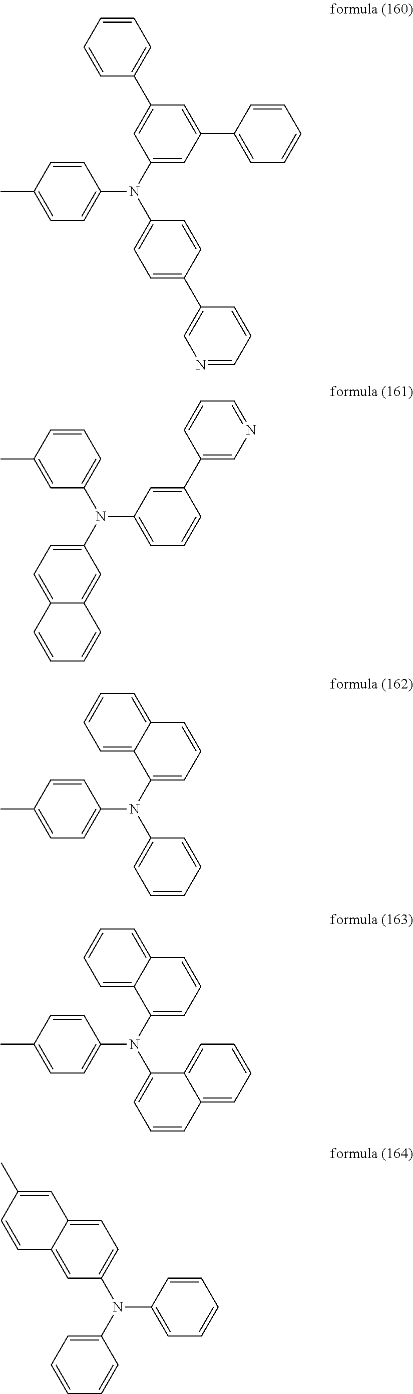

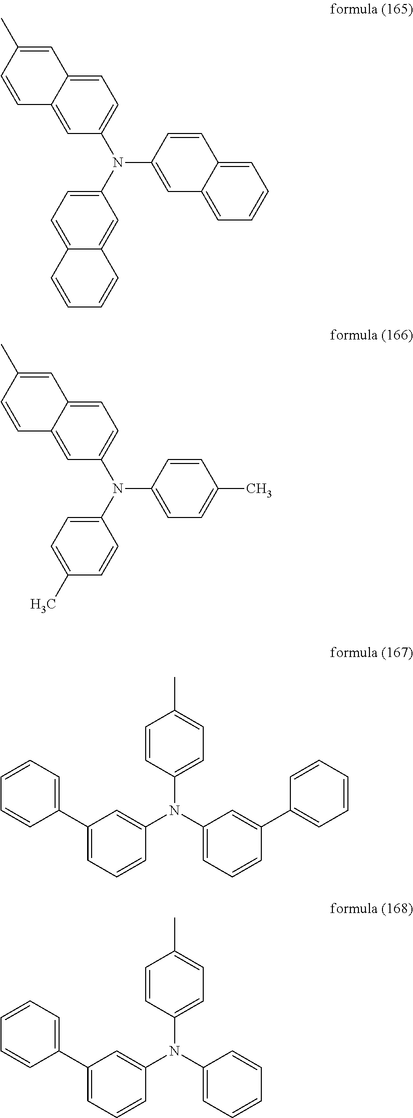 [SMILES: Cc1ccc2cc(N(c3ccc4ccccc4c3)c3ccc4ccccc4c3)ccc2c1 ; Cc1ccc(N(c2ccc(C)cc2)c2ccc3cc(C)ccc3c2)cc1 ; Cc1ccc(N(c2cccc(-c3ccccc3)c2)c2cccc(-c3ccccc3)c2)cc1 ; Cc1ccc(N(c2ccccc2)c2cccc(-c3ccccc3)c2)cc1]

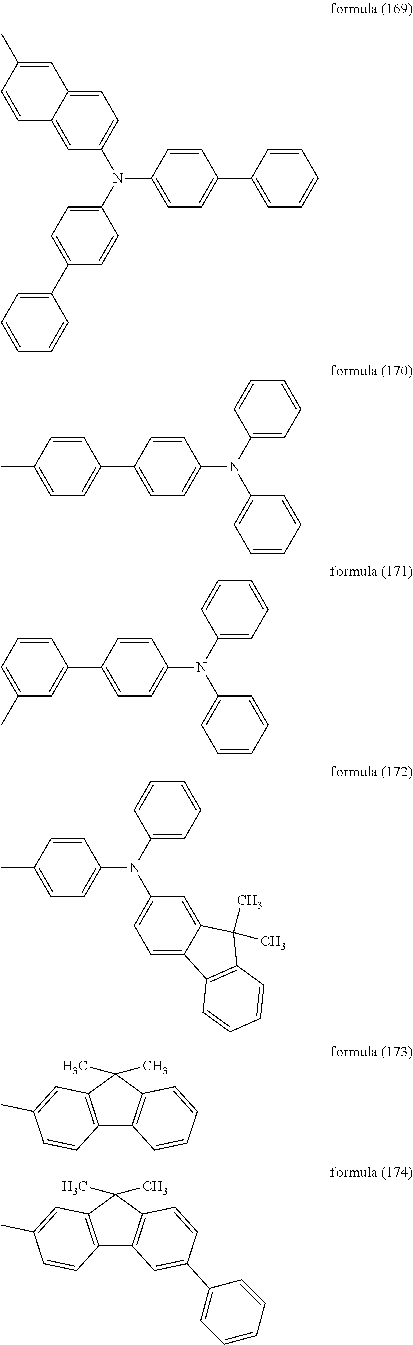

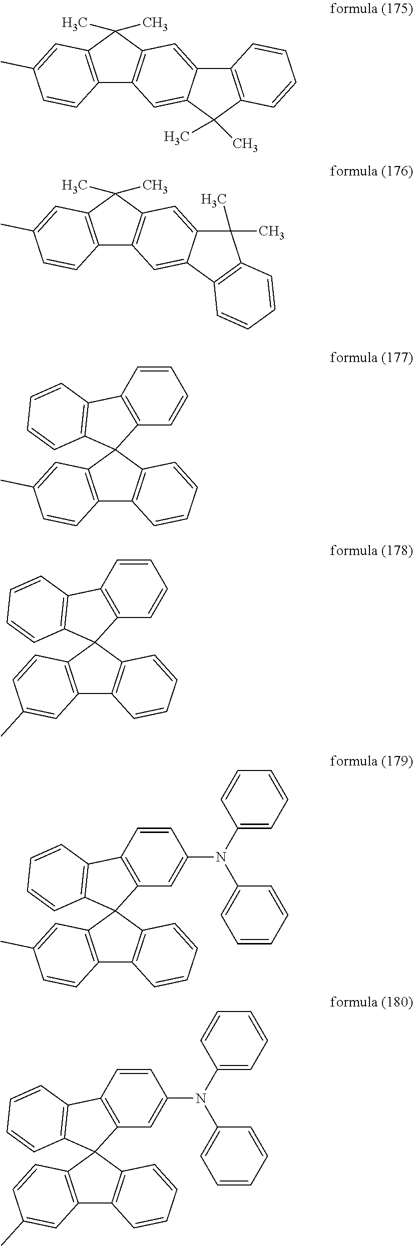

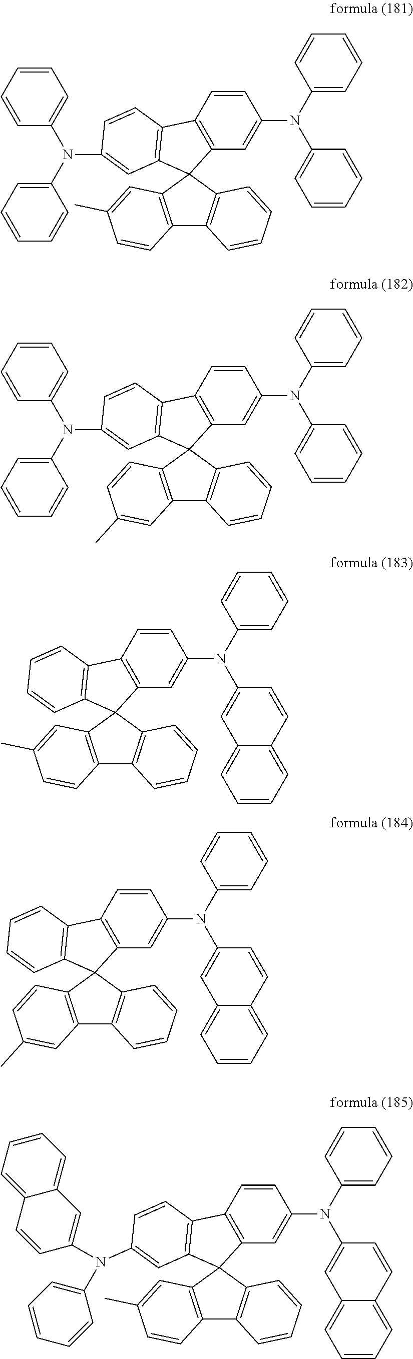

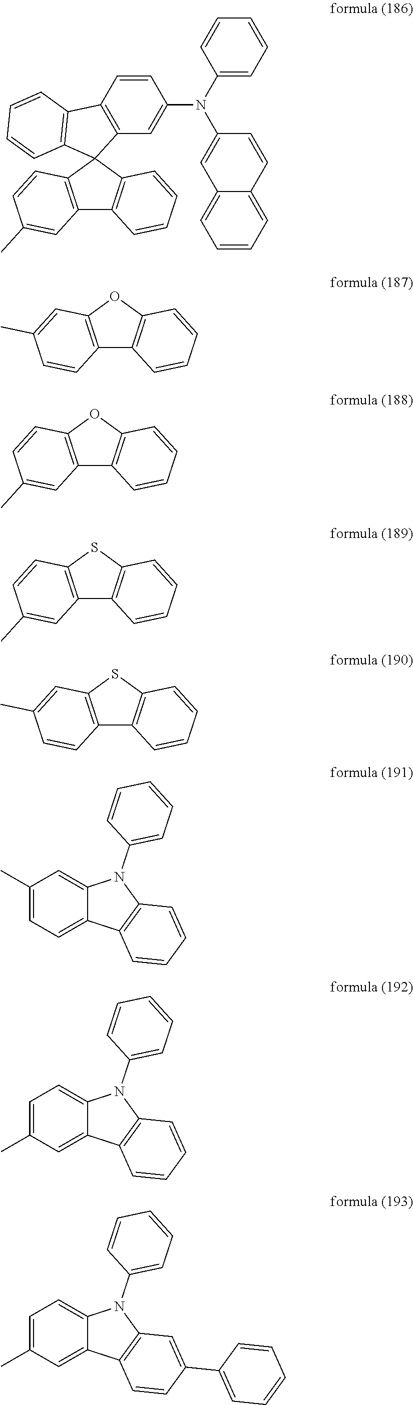

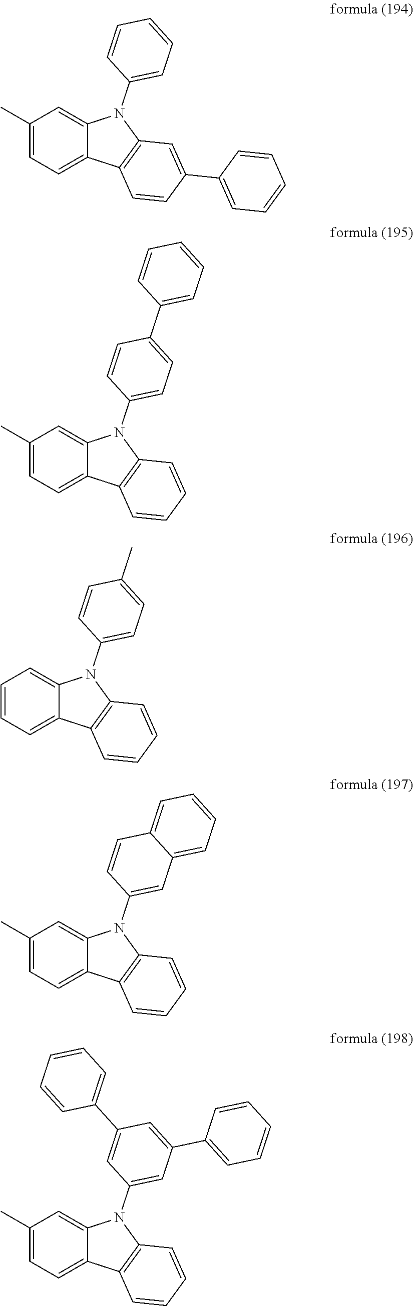

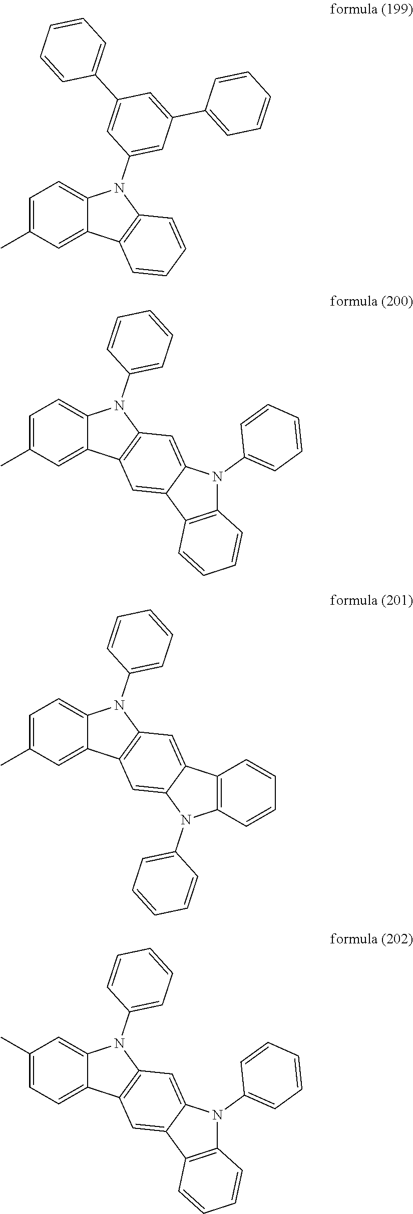

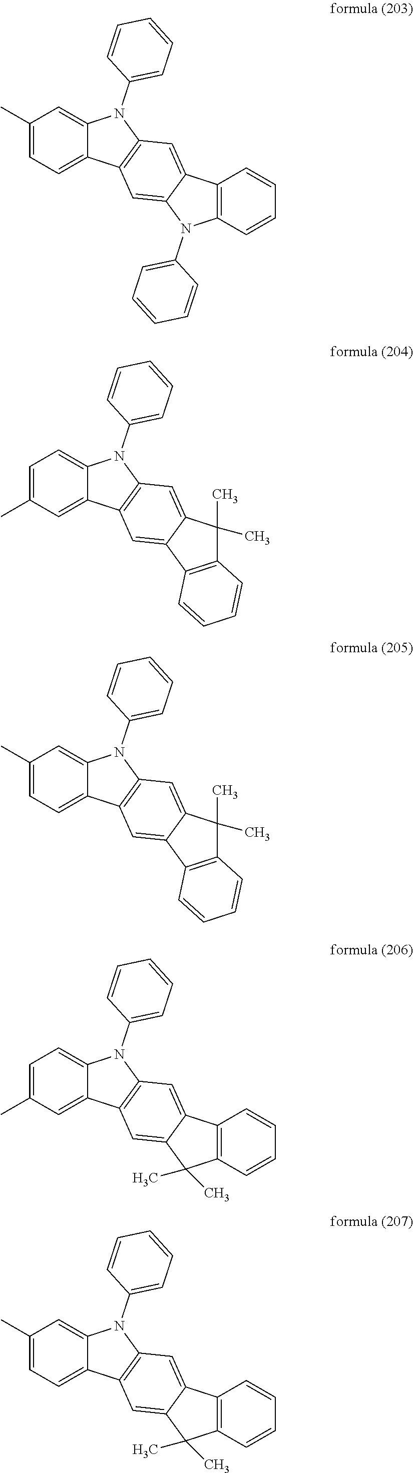

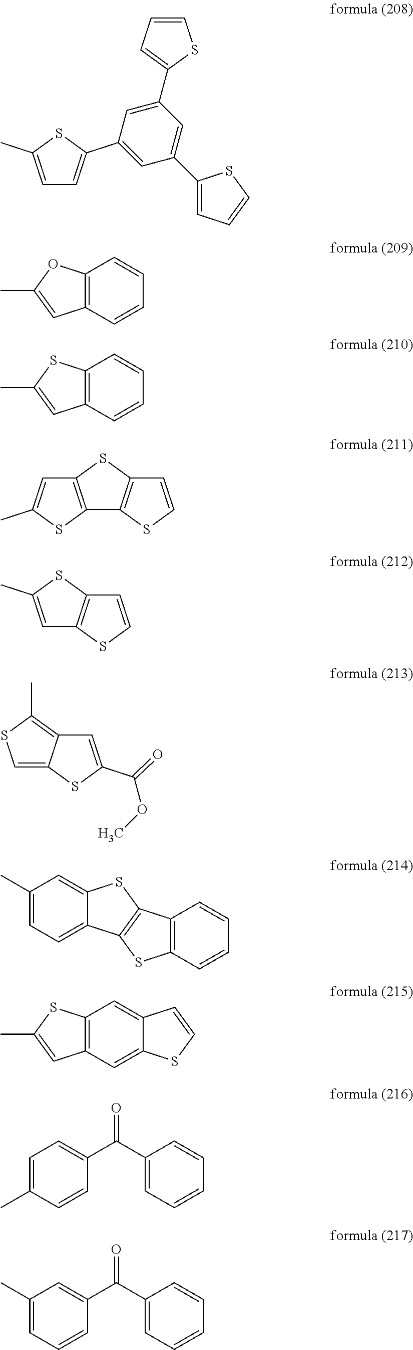

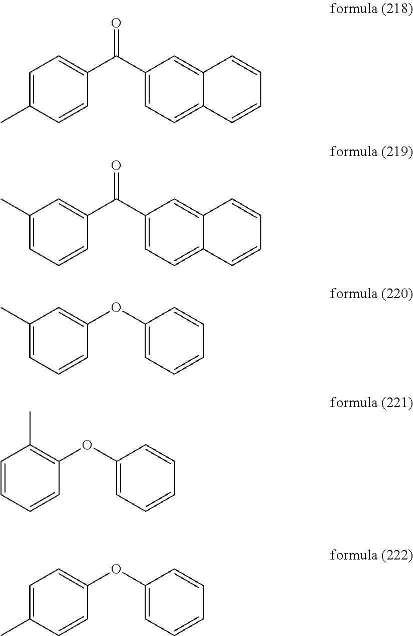

It is furthermore preferred for the purposes of the present invention for the sensitisers of the general formula (1) to have a symmetrical structure, i.e. for R¹ to be equal to Ar¹, with the proviso that both Ar¹ are now identical and each contain at least 9 ring atoms.

Some preferred compounds which can be used as sensitisers in composition for TTA-UpC in the sense of the present invention are disclosed below, without being limiting.

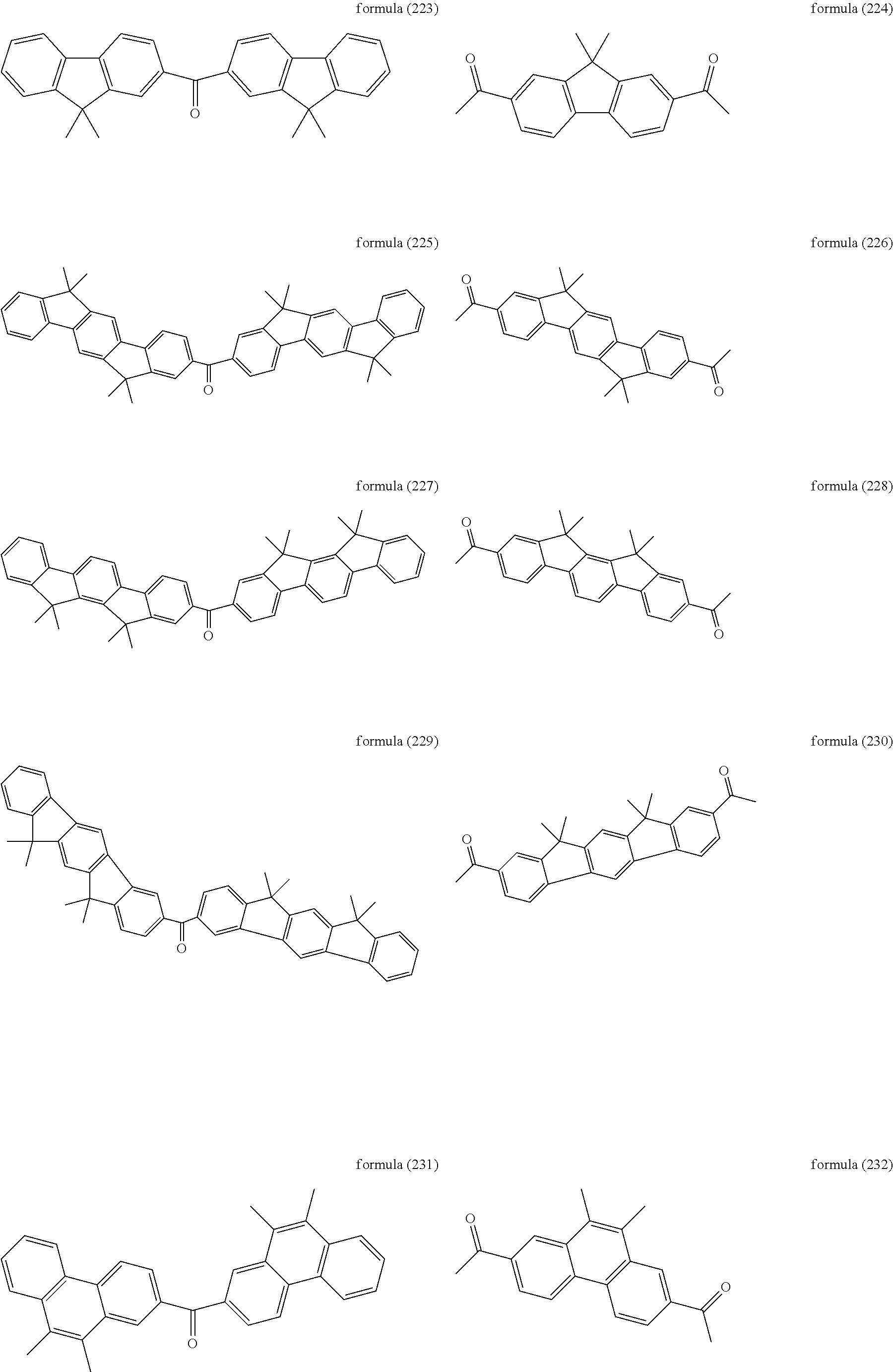

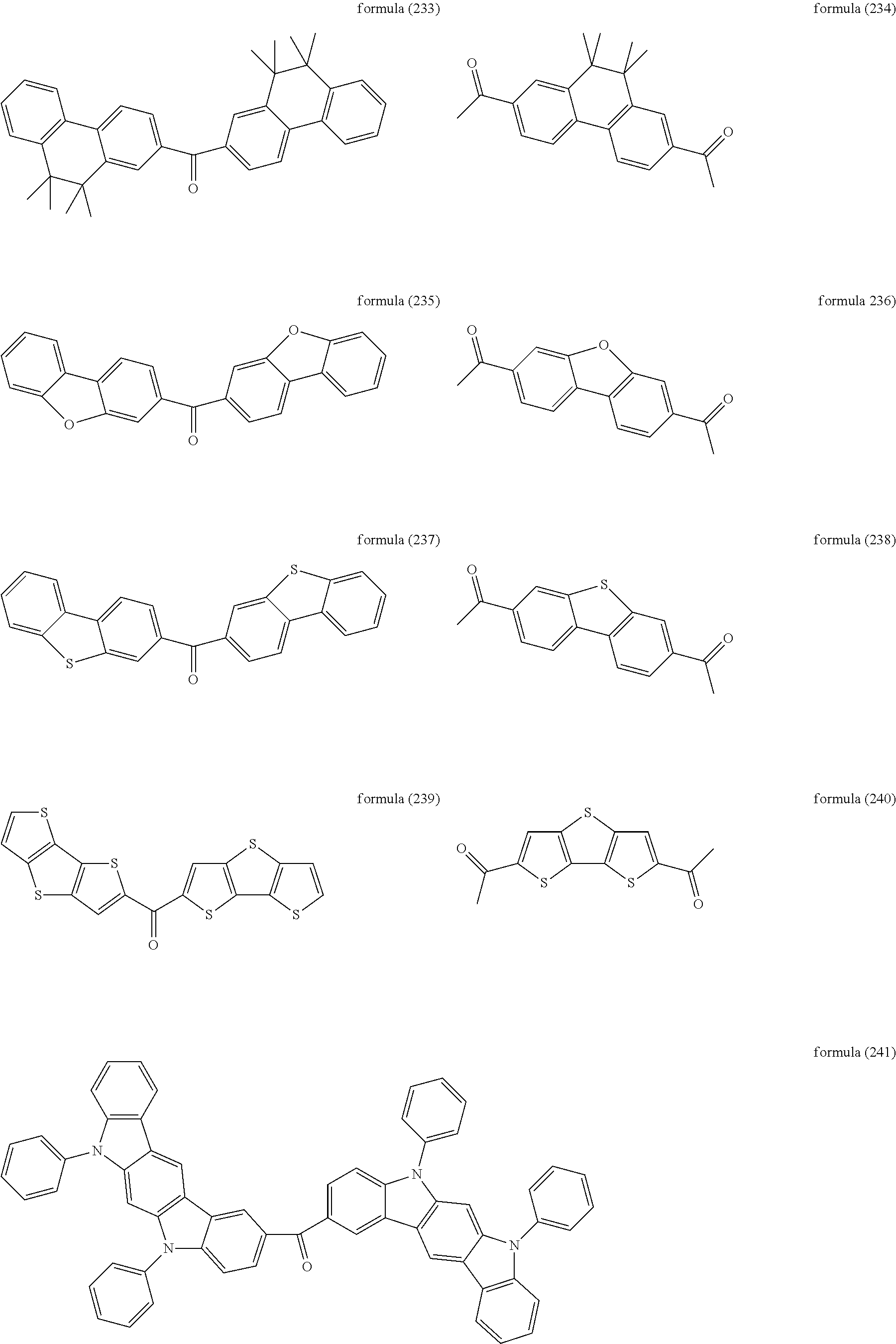

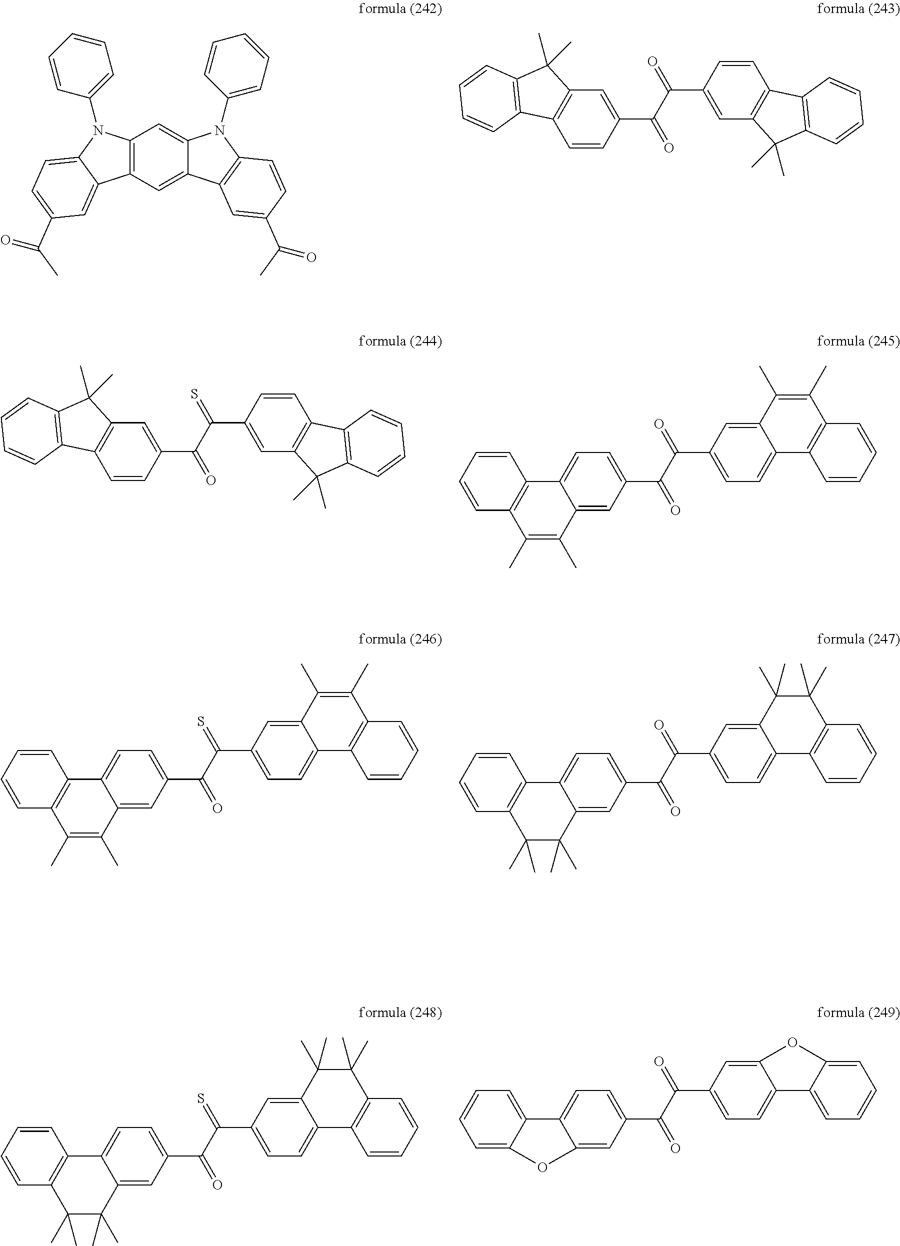

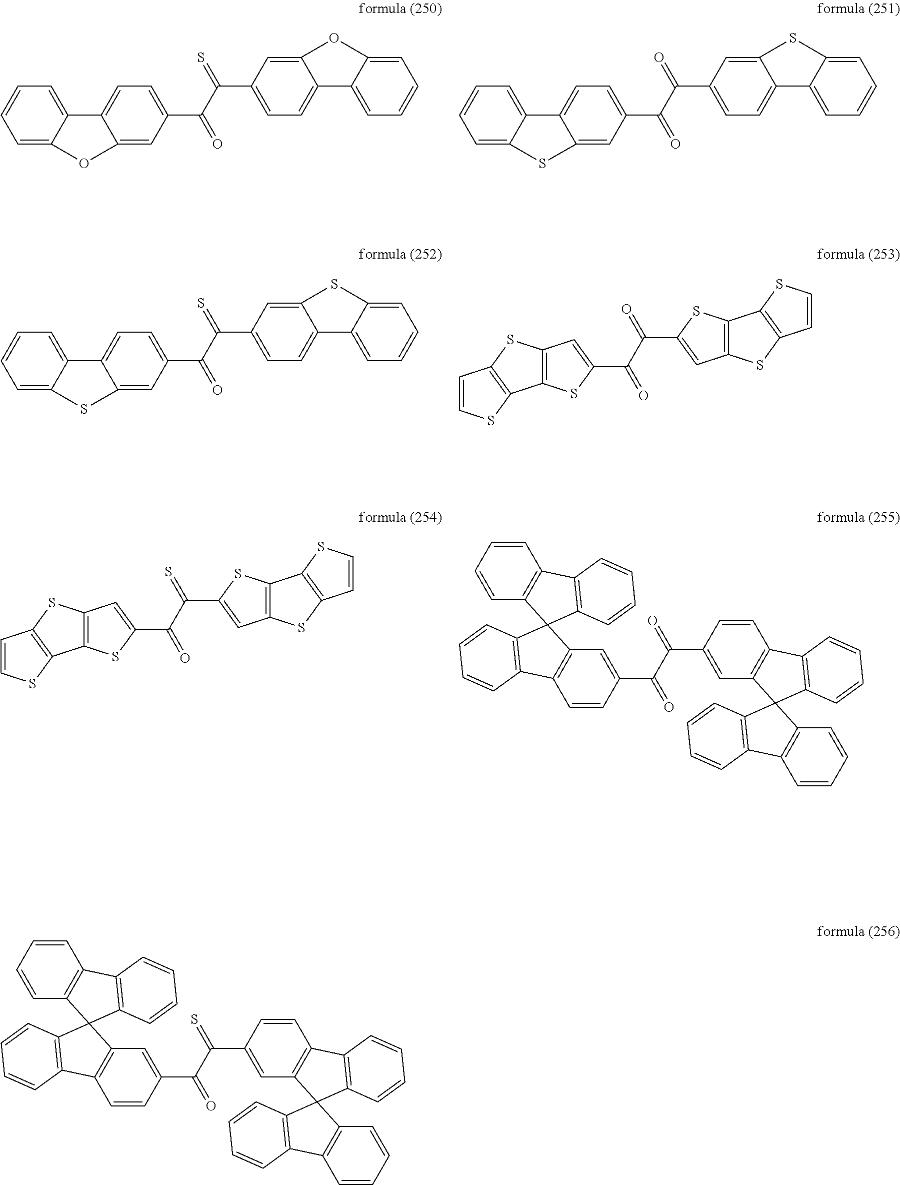

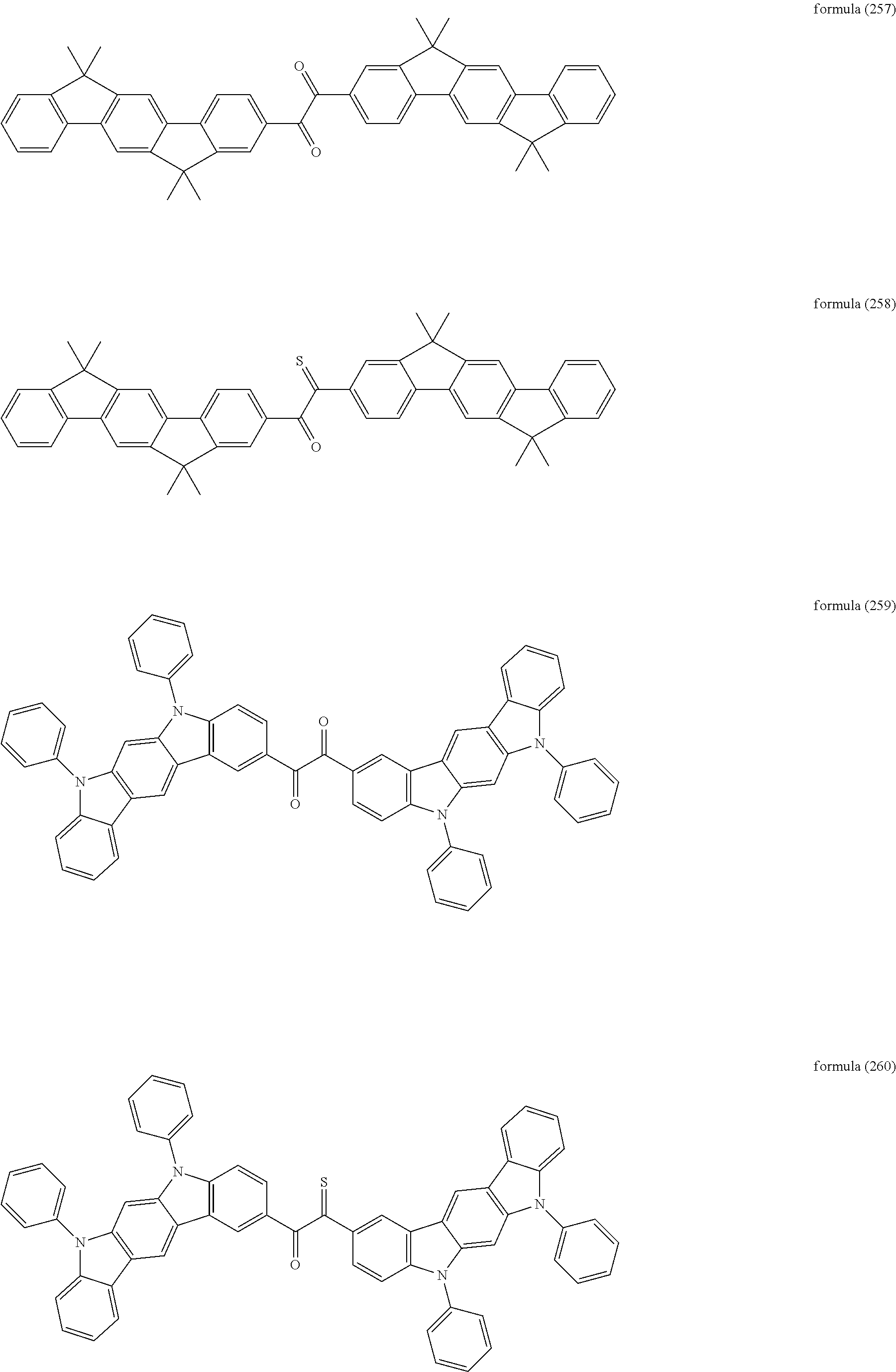 [SMILES: CC1(C)c2ccccc2-c2cc3c(cc21)-c1ccc(C(=O)C(=O)c2ccc4c(c2)C(C)(C)c2cc5c(cc2-4)C(C)(C)c2ccccc2-5)cc1C3(C)C ; CC1(C)c2ccccc2-c2cc3c(cc21)-c1ccc(C(=O)C(=S)c2ccc4c(c2)C(C)(C)c2cc5c(cc2-4)C(C)(C)c2ccccc2-5)cc1C3(C)C ; O=C(C(=O)c1ccc2c(c1)c1cc3c4ccccc4n(-c4ccccc4)c3cc1n2-c1ccccc1)c1ccc2c(c1)c1cc3c4ccccc4n(-c4ccccc4)c3cc1n2-c1ccccc1 ; O=C(C(=S)c1ccc2c(c1)c1cc3c4ccccc4n(-c4ccccc4)c3cc1n2-c1ccccc1)c1ccc2c(c1)c1cc3c4ccccc4n(-c4ccccc4)c3cc1n2-c1ccccc1]

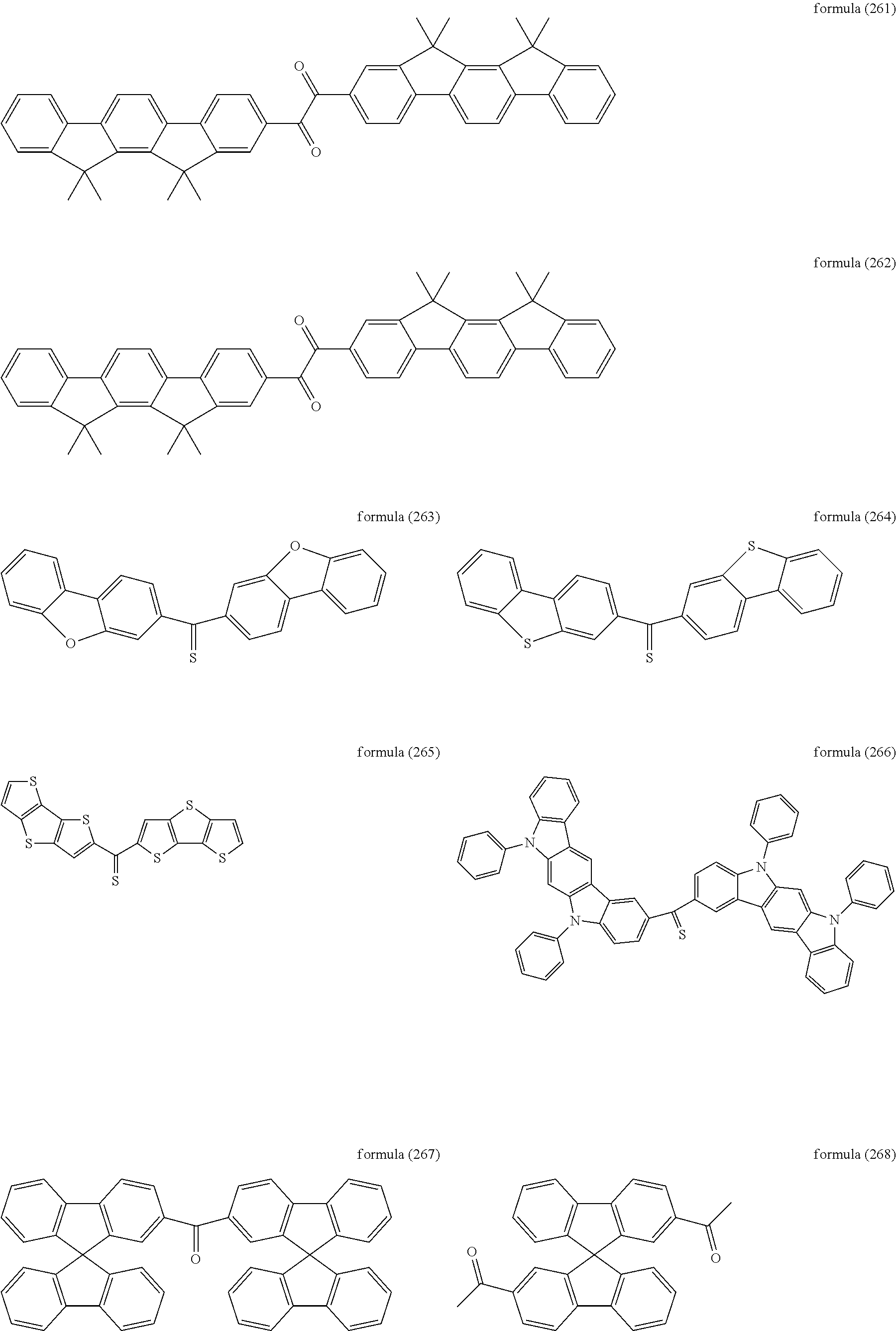

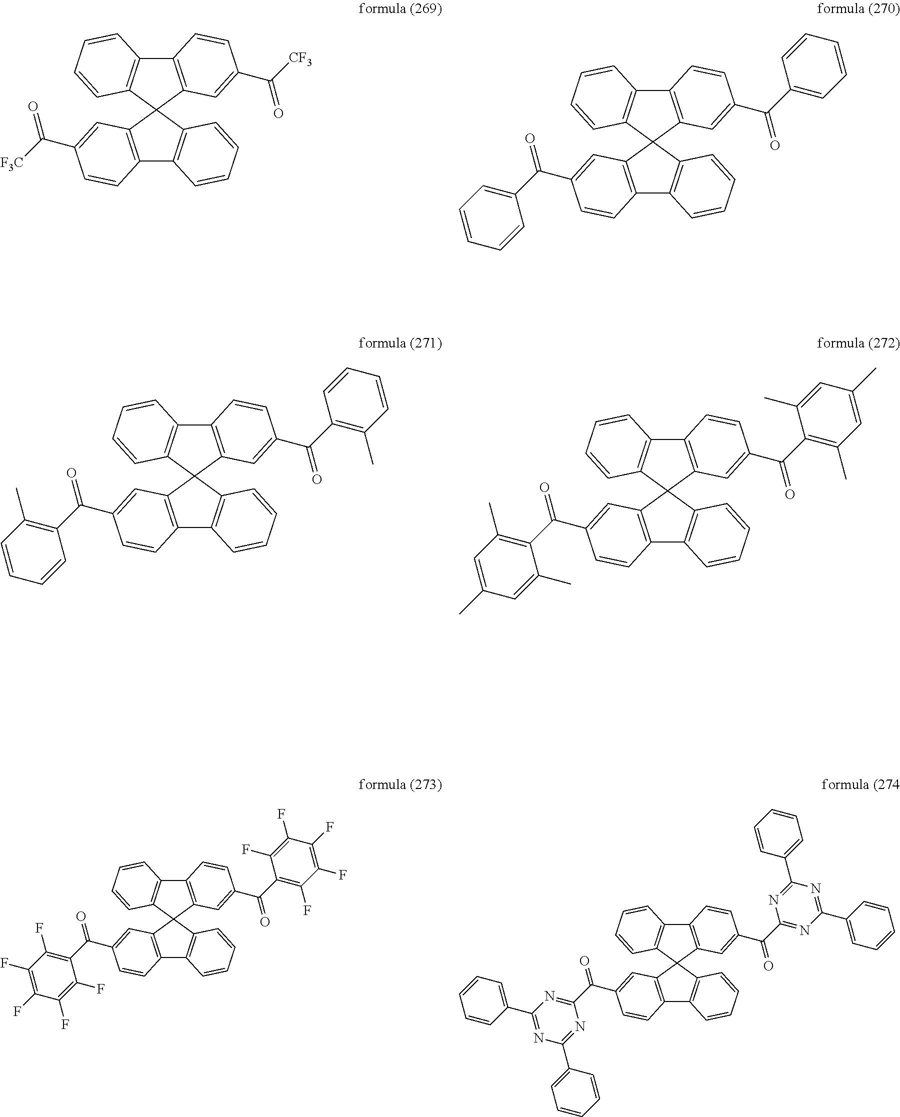

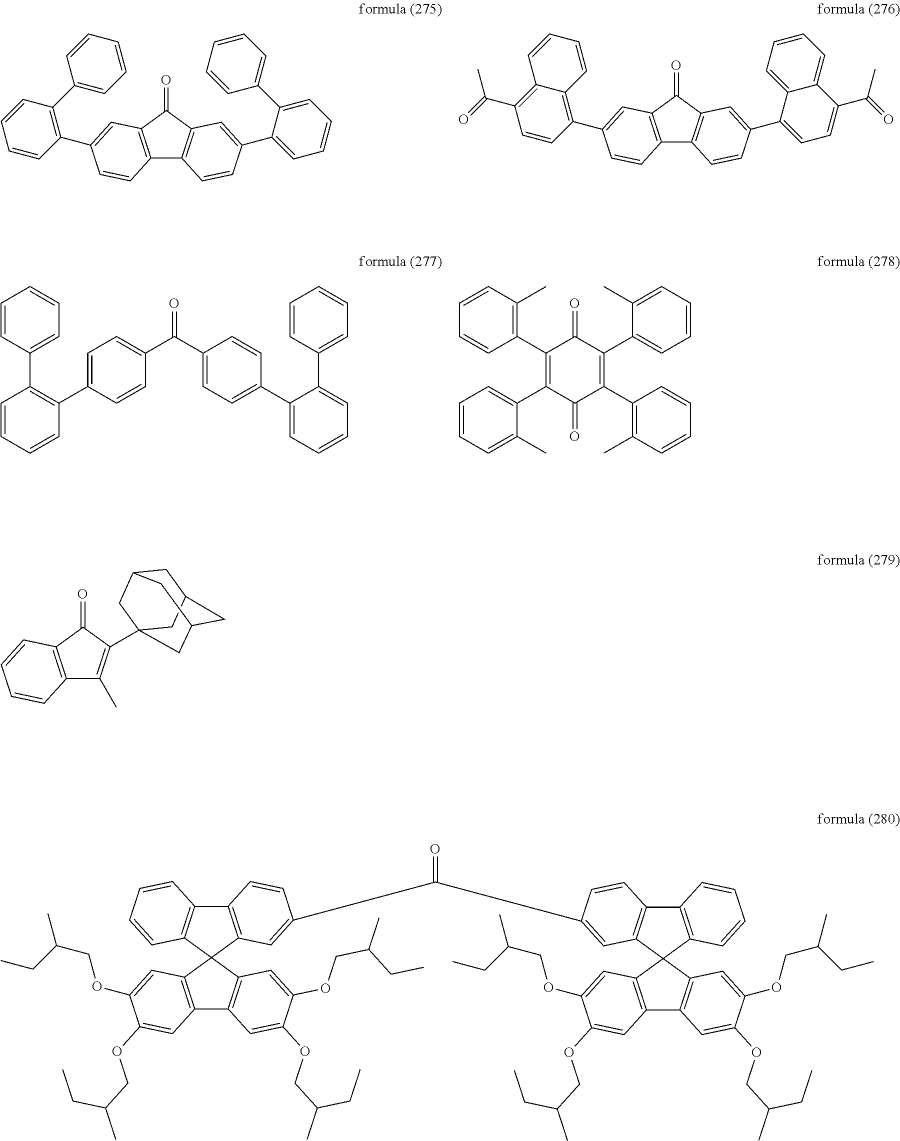 [SMILES: O=C1c2cc(-c3ccccc3-c3ccccc3)ccc2-c2ccc(-c3ccccc3-c3ccccc3)cc21 ; CC(=O)c1ccc(-c2ccc3c(c2)C(=O)c2cc(-c4ccc(C(C)=O)c5ccccc45)ccc2-3)c2ccccc12 ; O=C(c1ccc(-c2ccccc2-c2ccccc2)cc1)c1ccc(-c2ccccc2-c2ccccc2)cc1 ; Cc1ccccc1C1=C(c2ccccc2C)C(=O)C(c2ccccc2C)=C(c2ccccc2C)C1=O ; CC1=C(C23CC4CC(CC(C4)C2)C3)C(=O)c2ccccc21 ; CCC(C)COc1cc2c(cc1OCC(C)CC)C1(c3ccccc3-c3ccc(C(=O)c4ccc5c(c4)C4(c6ccccc6-5)c5cc(OCC(C)CC)c(OCC(C)CC)cc5-c5cc(OCC(C)CC)c(OCC(C)CC)cc54)cc31)c1cc(OCC(C)CC)c(OCC(C)CC)cc1-2]

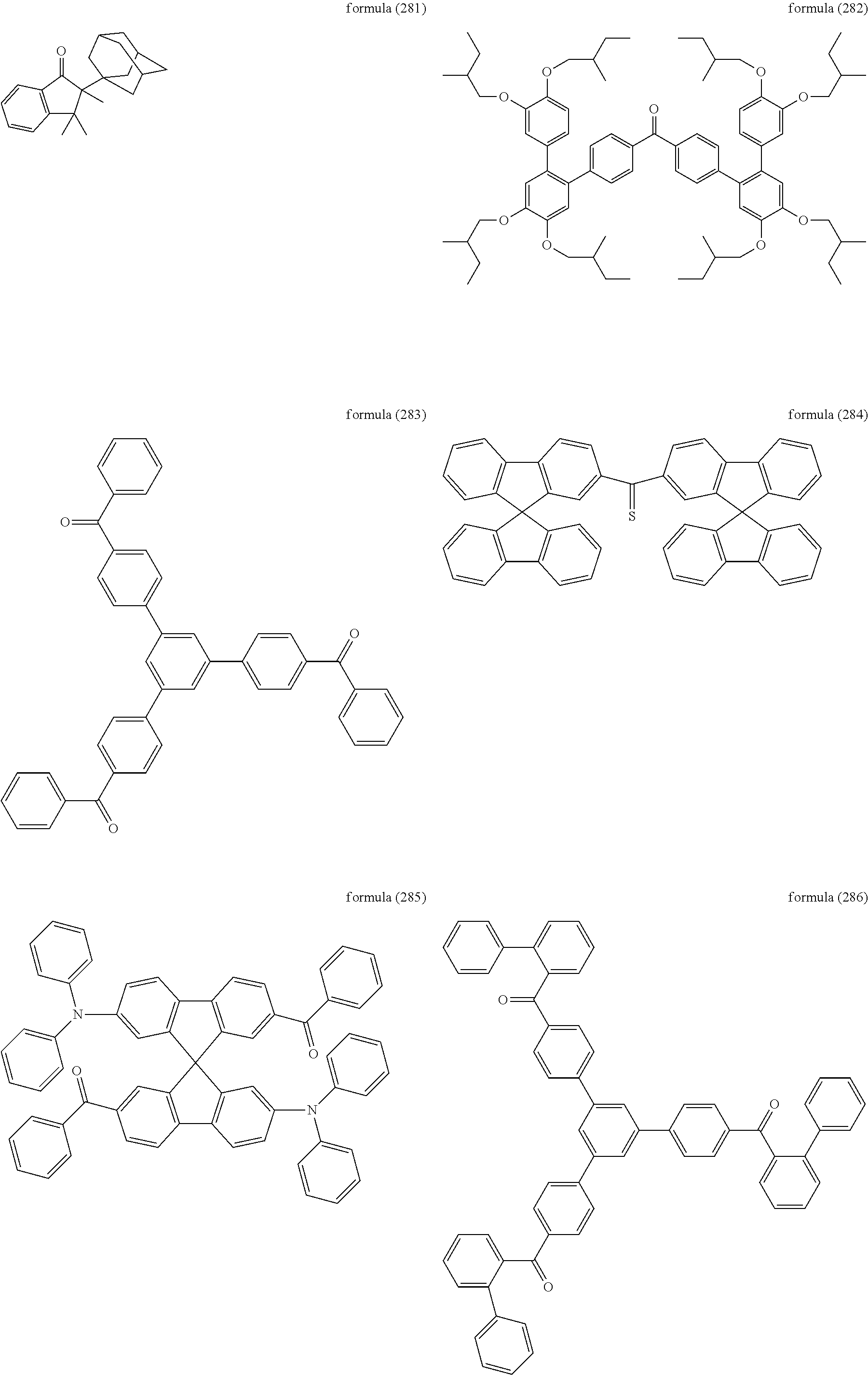

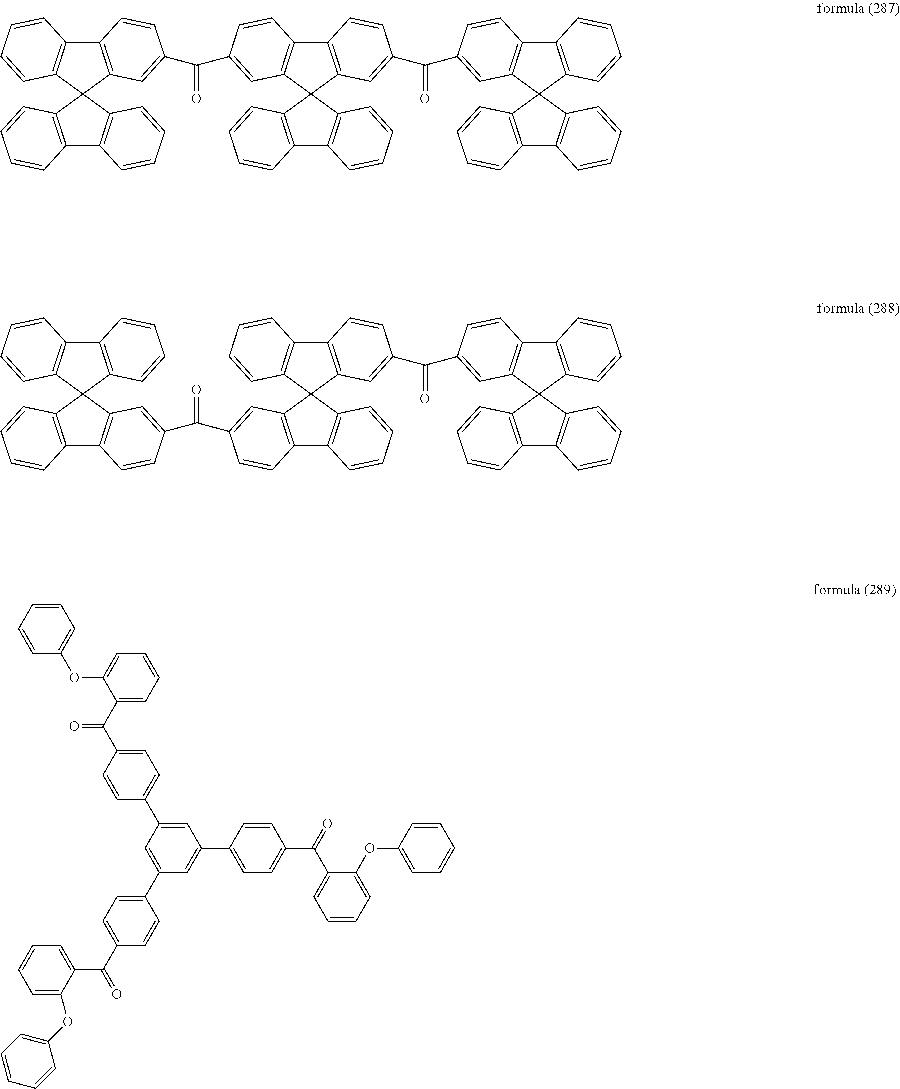 [SMILES: O=C(c1ccc2c(c1)C1(c3ccccc3-c3ccccc31)c1ccccc1-2)c1ccc2c(c1)C1(c3ccccc3-c3ccccc31)c1cc(C(=O)c3ccc4c(c3)C3(c5ccccc5-c5ccccc53)c3ccccc3-4)ccc1-2 ; O=C(c1ccc2c(c1)C1(c3ccccc3-c3ccccc31)c1ccccc1-2)c1ccc2c(c1)C1(c3ccccc3-2)c2ccccc2-c2ccc(C(=O)c3ccc4c(c3)C3(c5ccccc5-c5ccccc53)c3ccccc3-4)cc21 ; O=C(c1ccc(-c2cc(-c3ccc(C(=O)c4ccccc4Oc4ccccc4)cc3)cc(-c3ccc(C(=O)c4ccccc4Oc4ccccc4)cc3)c2)cc1)c1ccccc1Oc1ccccc1]

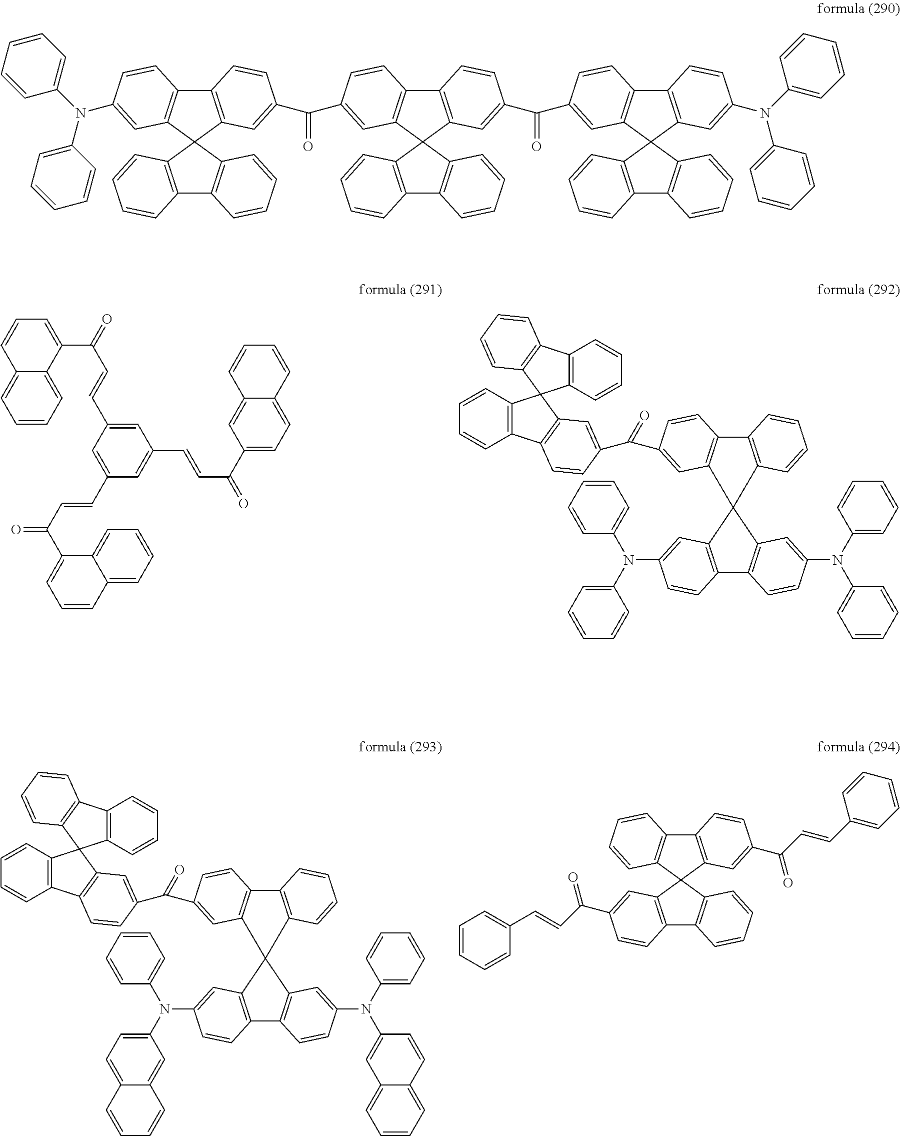

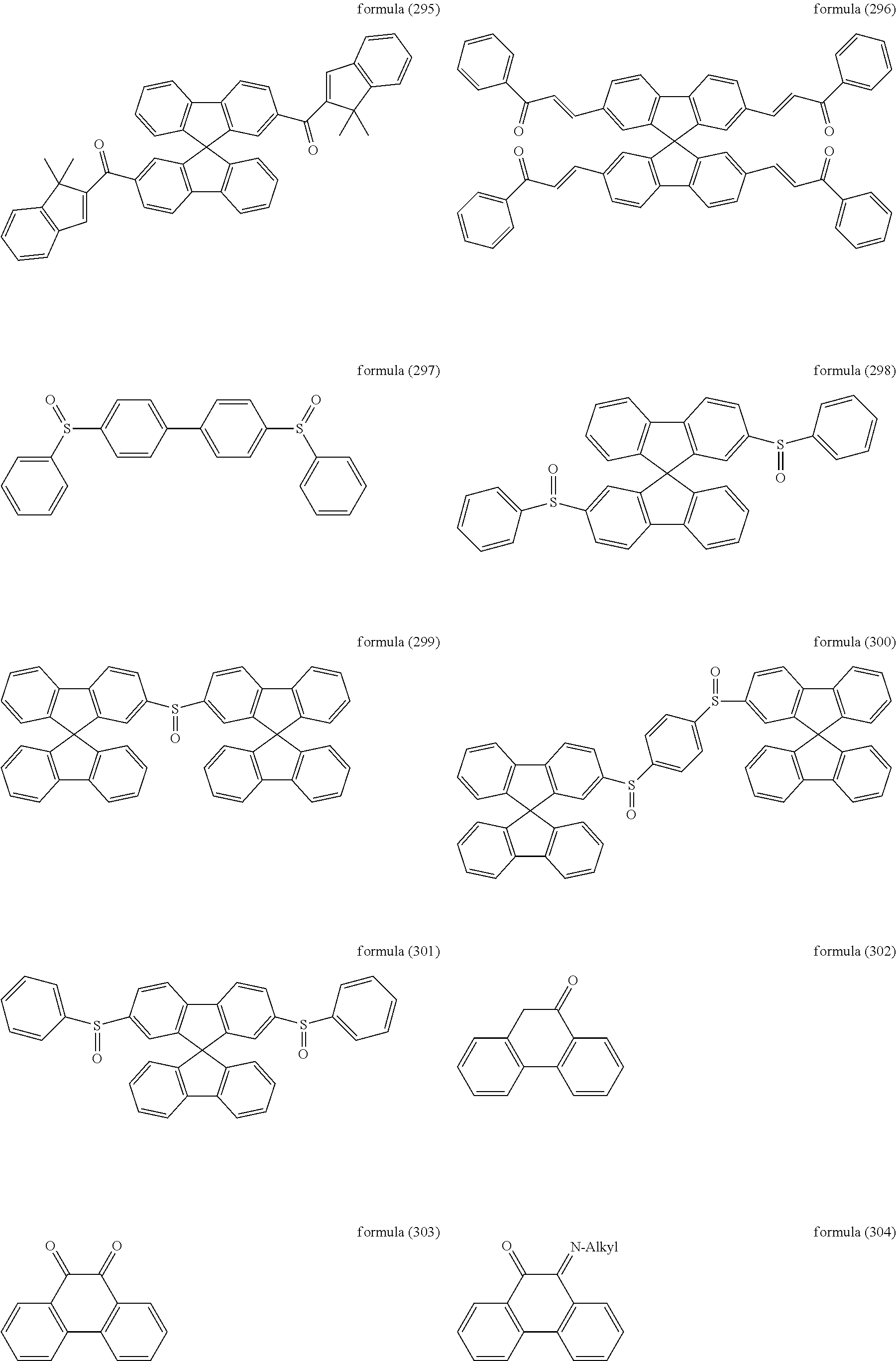

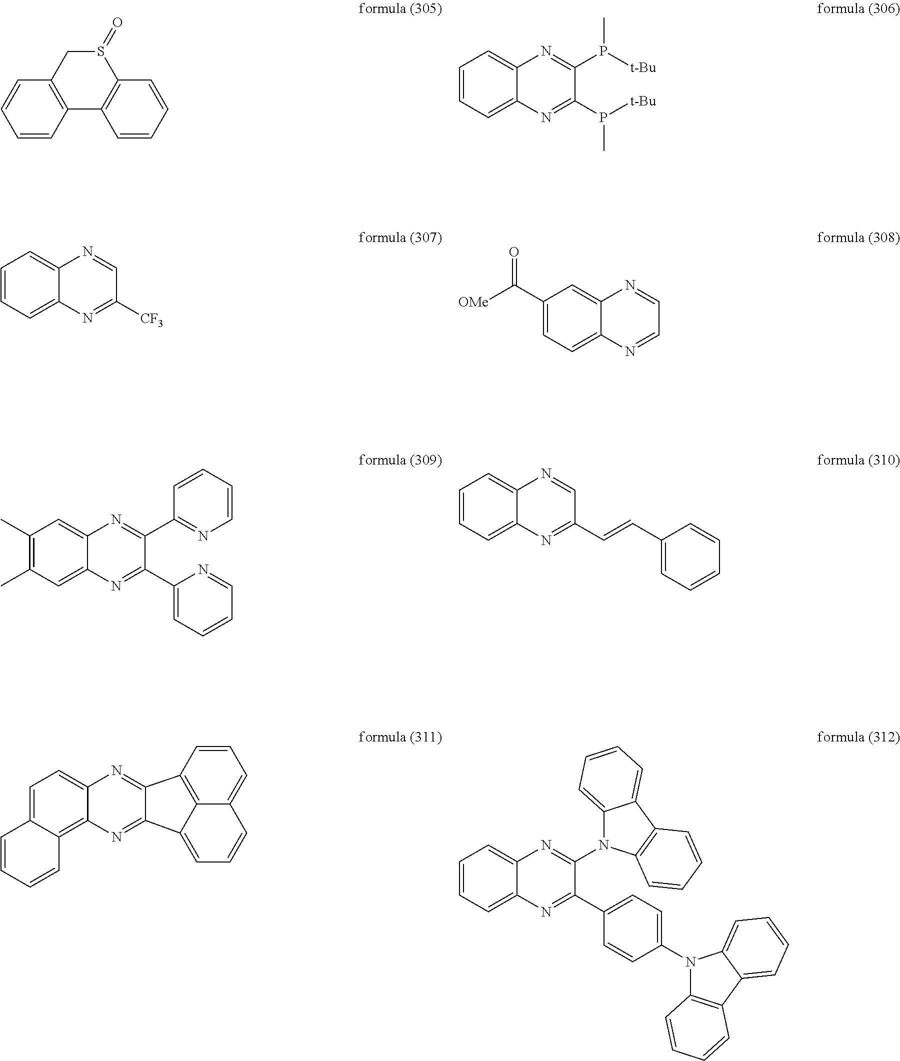

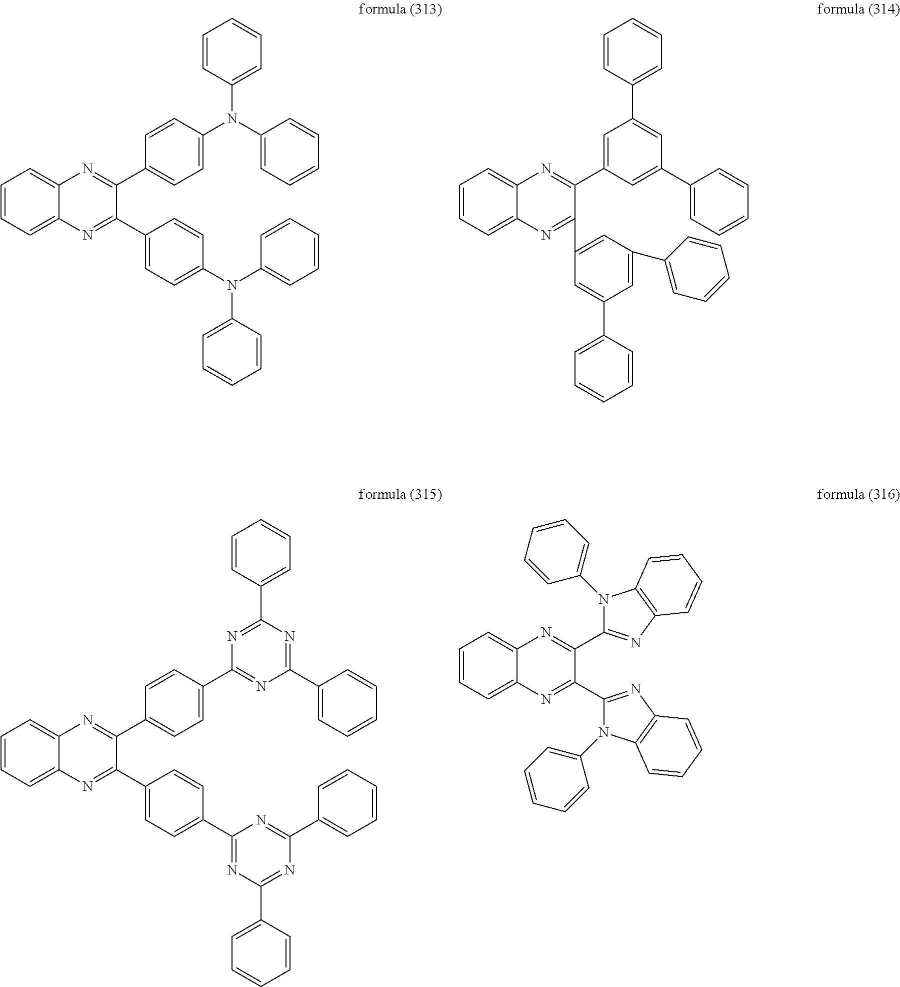 [SMILES: c1ccc(N(c2ccccc2)c2ccc(-c3nc4ccccc4nc3-c3ccc(N(c4ccccc4)c4ccccc4)cc3)cc2)cc1 ; c1ccc(-c2cc(-c3ccccc3)cc(-c3nc4ccccc4nc3-c3cc(-c4ccccc4)cc(-c4ccccc4)c3)c2)cc1 ; c1ccc(-c2nc(-c3ccccc3)nc(-c3ccc(-c4nc5ccccc5nc4-c4ccc(-c5nc(-c6ccccc6)nc(-c6ccccc6)n5)cc4)cc3)n2)cc1 ; c1ccc(-n2c(-c3nc4ccccc4nc3-c3nc4ccccc4n3-c3ccccc3)nc3ccccc32)cc1]

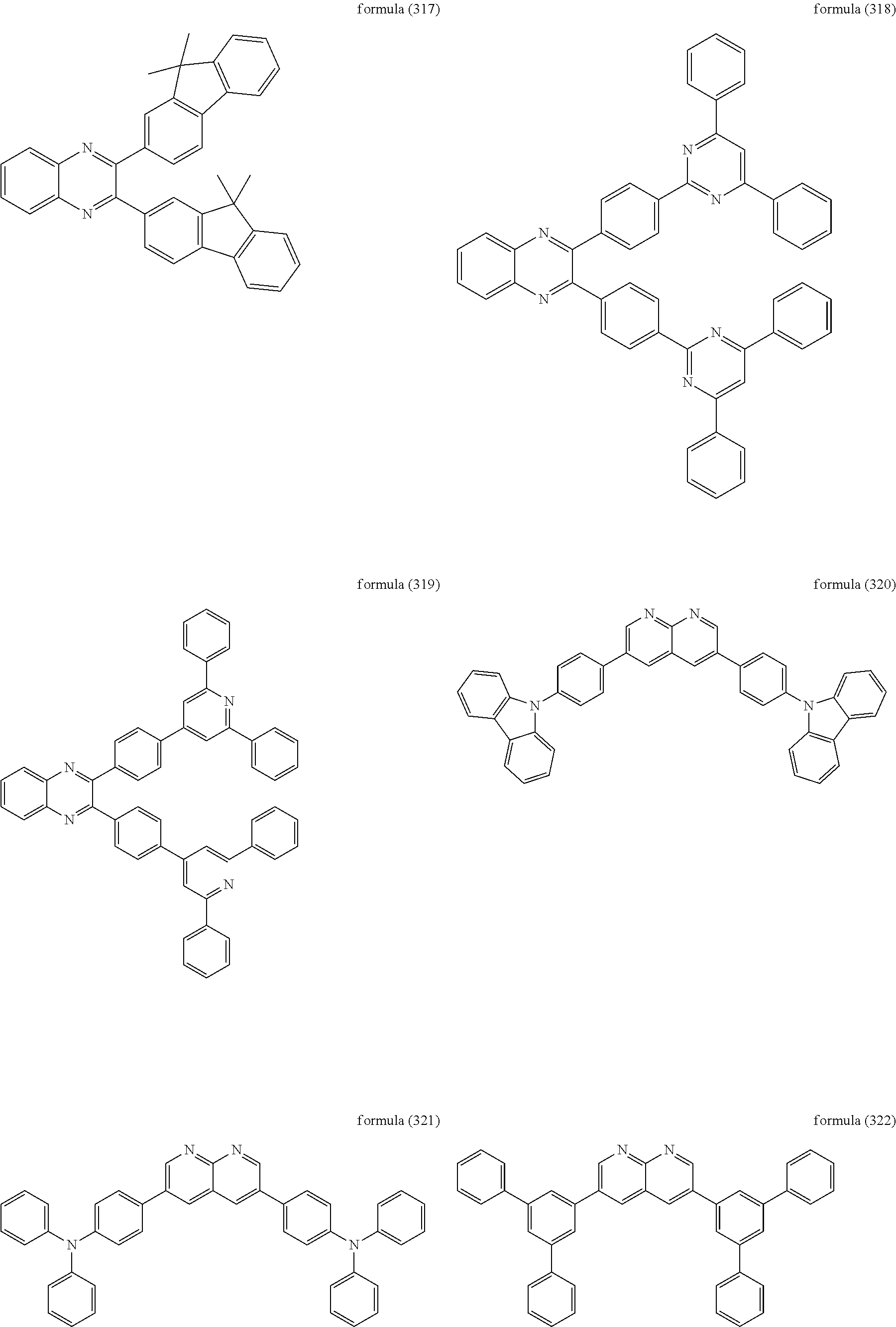

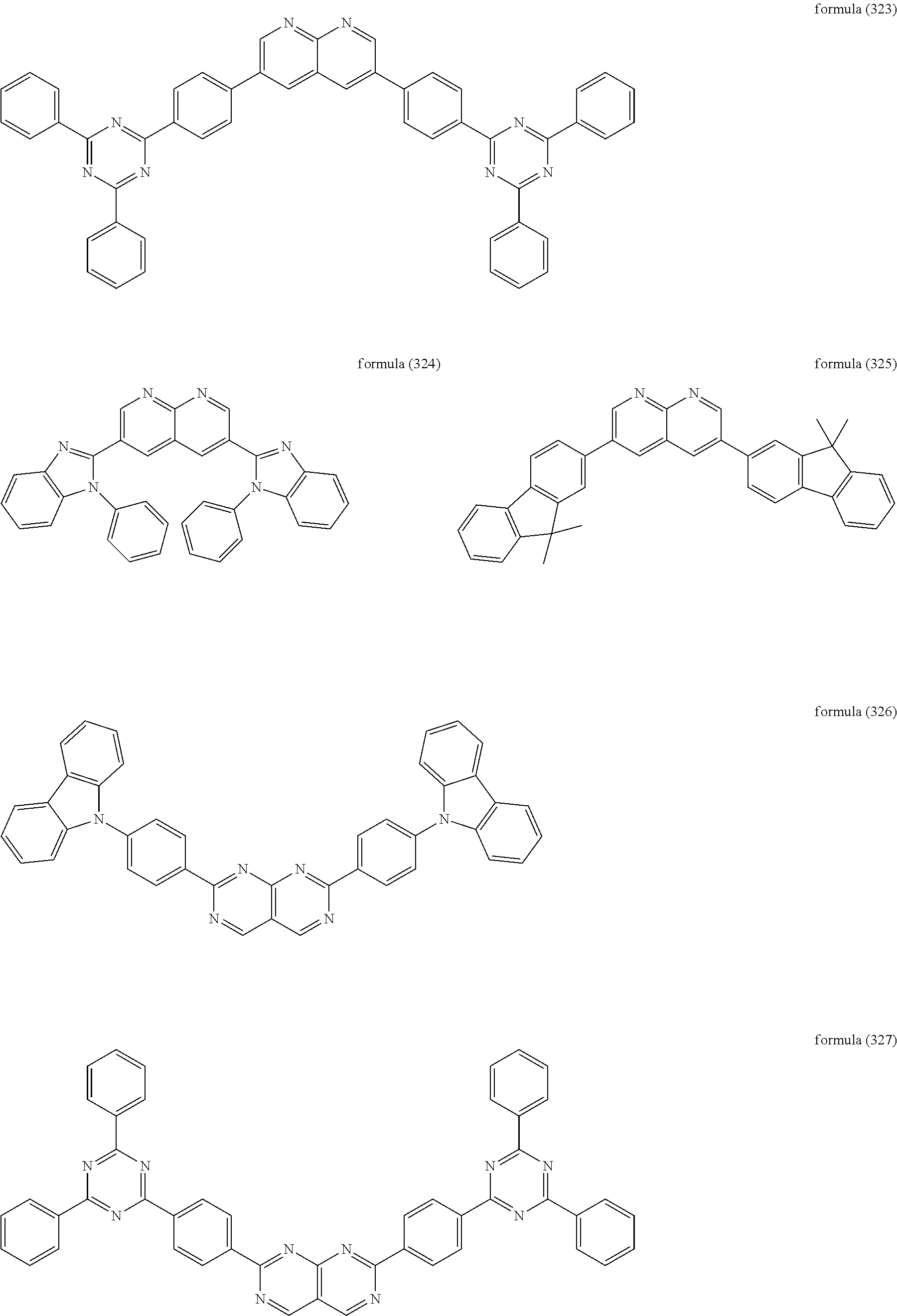 [SMILES: c1ccc(-c2nc(-c3ccccc3)nc(-c3ccc(-c4cnc5ncc(-c6ccc(-c7nc(-c8ccccc8)nc(-c8ccccc8)n7)cc6)cc5c4)cc3)n2)cc1 ; c1ccc(-n2c(-c3cnc4ncc(-c5nc6ccccc6n5-c5ccccc5)cc4c3)nc3ccccc32)cc1 ; CC1(C)c2ccccc2-c2ccc(-c3cnc4ncc(-c5ccc6c(c5)C(C)(C)c5ccccc5-6)cc4c3)cc21 ; c1ccc2c(c1)c1ccccc1n2-c1ccc(-c2ncc3cnc(-c4ccc(-n5c6ccccc6c6ccccc65)cc4)nc3n2)cc1 ; c1ccc(-c2nc(-c3ccccc3)nc(-c3ccc(-c4ncc5cnc(-c6ccc(-c7nc(-c8ccccc8)nc(-c8ccccc8)n7)cc6)nc5n4)cc3)n2)cc1]

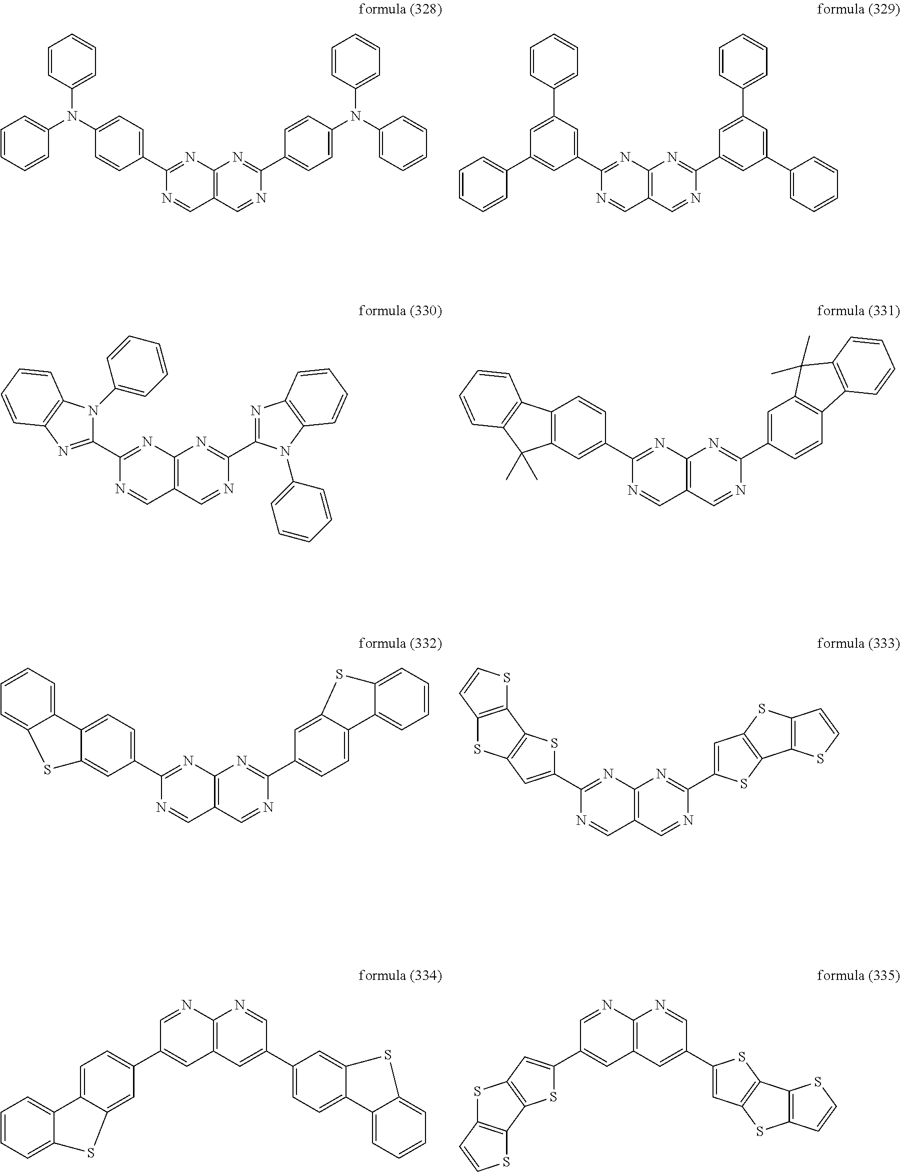

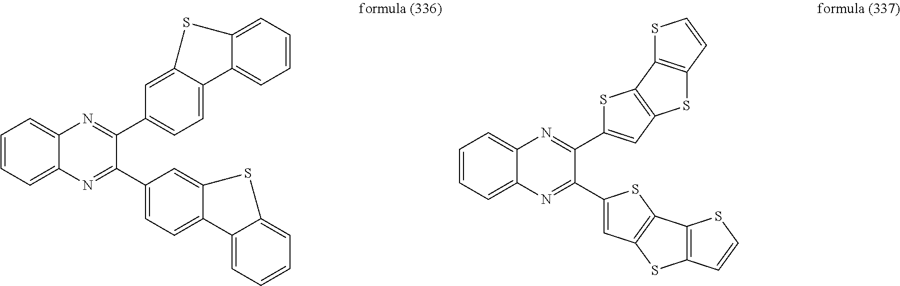 [SMILES: c1ccc2nc(-c3ccc4c(c3)sc3ccccc34)c(-c3ccc4c(c3)sc3ccccc34)nc2c1 ; c1ccc2nc(-c3cc4sc5ccsc5c4s3)c(-c3cc4sc5ccsc5c4s3)nc2c1]

Besides the at least one sensitiser, the compositions according to the invention comprise at least one fluorescent emitter which is not a metal complex.

Preference is given to a composition according to the invention comprising 3, very preferably 2 and very particularly preferably one sensitiser.

Preference is furthermore given to compositions according to the invention comprising 3, very preferably 2 and very particularly preferably one fluorescent emitter.

High preference is given to compositions according to the invention comprising 2 sensitisers and 3, preferably 2 and very preferably one fluorescent emitter.

Very particular preference is given to compositions according to the invention comprising one sensitiser and two fluorescent emitters.

Especial preference is given to compositions according to the invention comprising one sensitiser and one fluorescent emitter.

The proportion by weight of the sensitiser in the composition according to the invention is 1.0% by weight to 97% by weight, preferably 5% by weight to 95% by weight, very preferably 10% by weight to 93% by weight, and very particularly preferably 20% by weight to 93% by weight.

Fluorescent emitters which can be employed in the compositions and devices according to the invention for TTA-UpC are described as follows.

In a preferred embodiment, the emitter is a blue or UV emitter.

In the context of the present invention, the terms singlet emitters, singlet dopants, fluorescent emitters and fluorescent dopants have the same meaning.

Suitable dopants are selected from the class of the monostyrylamines, the distyrylamines, the tristyrylamines, the tetrastyrylamines, the styrylphosphines, the styryl ethers and the arylamines. A monostyrylamine is taken to mean a compound which contains one substituted or unsubstituted styryl group and at least one, preferably aromatic, amine. A distyrylamine is taken to mean a compound which contains two substituted or unsubstituted styryl groups and at least one, preferably aromatic, amine. A tristyrylamine is taken to mean a compound which contains three substituted or unsubstituted styryl groups and at least one, preferably aromatic, amine. A tetrastyrylamine is taken to mean a compound which contains four substituted or unsubstituted styryl groups and at least one, preferably aromatic, amine. The styryl groups are particularly preferably stilbenes, which may also be further substituted. Corresponding phosphines and ethers are defined analogously to the amines. An arylamine or aromatic amine in the sense of this invention is taken to mean a compound which contains three substituted or unsubstituted aromatic or heteroaromatic ring systems bonded directly to the nitrogen. At least one of these aromatic or heteroaromatic ring systems is preferably a condensed ring system, preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 2- or 9-position. An aromatic anthracenediamine is taken to mean a compound in which two diarylamino groups are bonded directly to an anthracene group, preferably in the 2,6- or 9,10-position. Aromatic pyrenamines, pyrenediamines, chrysenamines and chrysenediamines are defined analogously thereto, Where the diarylamino groups are preferably bonded to the pyrene in the 1-position or in the 1,6-position. Further preferred dopants are selected from indenofluorenamines or indenofluorenediamines, for example in accordance with WO 2006/122630, benzoindenofluorenamines or benzoindenofluorenediamines, for example in accordance with WO 2008/006449, and dibenzoindenofluorenamines or dibenzoindenofluorenediamines, for example in accordance with WO 2007/140847. Examples of dopants from the class of the styrylamines are substituted or unsubstituted tristilbenamines or the dopants described in the patent applications WO 2006/000388, WO 2006/058737, WO 2006/000389, WO 2007/065549 and WO 2007/115610. Preference is furthermore given to bridged aromatic hydrocarbons, such as, for example, the compounds disclosed in WO 2010/012328.

Preferred fluorescent dopants are the compounds of the following formulae (338) and (339)

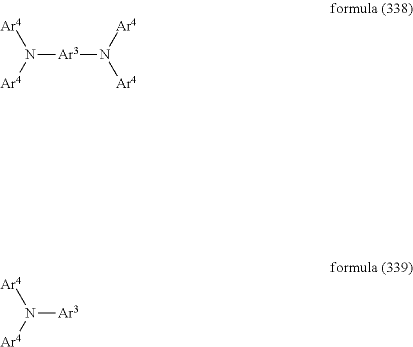

where the following applies to the symbols used:
Ar³ is a condensed aryl or heteroaryl group or a condensed aromatic or heteroaromatic ring system having 10 to 40 aromatic ring atoms, which may be substituted by one or more radicals R²;
Ar⁴ is on each occurrence, identically or differently, an aromatic or heteroaromatic ring system having 5 to 30 aromatic ring atoms, which may be substituted by one or more radicals R⁴; two radicals Ar⁴ here which are bonded to the same nitrogen atom may also be linked to one another by a single bond or a bridge selected from B(R⁴), C(R⁴)₂, Si(R⁴)₂, C═O, C═NR⁴, C═C(R⁴)₂, O, S, S═O, SO₂, N(R⁴), P(R⁴) and P(═O) R⁴;
R⁴ is on each occurrence, identically or differently, H, D, F, Cl, Br, I, CHO, N(R⁵)₂, C(═O)R⁵, P(═O)(R⁵)₂, S(═O)R⁵, S(═O)₂R⁵, CR⁵═C(R⁵)₂, CN, NO₂, Si(R⁵)₃, B(OR⁵)₂, B(R⁵)₂, B(N(R⁵)₂)₂, OSO₂R⁵, a straight-chain alkyl, alkoxy or thioalkoxy group having 1 to 40 C atoms or a branched or cyclic alkyl, alkoxy or thioalkoxy group having 3 to 40 C atoms or an alkenyl or alkynyl group having 2 to 40 C atoms, where the alkyl, alkoxy, thioalkoxy, alkenyl or alkynyl group may in each case be substituted by one or more radicals R⁵, where one or more non-adjacent CH₂ groups may be replaced by R⁵C═CR⁵, C≡C, Si(R⁵)₂, C═O, C═S, C═NR⁵, P(═O)(R⁵), SO, SO₂, NR⁵, O, S or CONR⁵ and where one or more H atoms may be replaced by D, F, CI, Br, I, CN or NO₂, or an aromatic or heteroaromatic ring system having 5 to 30 aromatic ring atoms, which may in each case be substituted by one or more radicals R⁵, or an aryloxy or heteroaryloxy group having 5 to 30 aromatic ring atoms, Which may be substituted by one or more radicals R⁵; two or more adjacent substituents R⁵ here may also form a mono- or polycyclic, aliphatic or aromatic ring system with one another;
R⁵ is on each occurrence, identically or differently, H, D or an aliphatic, aromatic and/or heteroaromatic hydrocarbon radical having 1 to 20 C atoms, in which, in addition, H atoms may be replaced by D, CN or F; two or more adjacent substituents R⁵ here may also form a mono- or polycyclic, aliphatic or aromatic ring system with one another.

In a preferred embodiment of the invention, Ar³ is a condensed aryl group or a condensed aromatic ring system. Preferred condensed aryl groups or aromatic ring systems Ar³ are selected from the group consisting of anthracene, pyrene, fluoranthene, naphthacene, chrysene, benzanthracene, benzofluorene, triphenylene, perylene, cis- or trans-monobenzoindenofluorene and cis- or trans-dibenzoindenofluorene, each of which may be substituted by one or more radicals R⁴.

In a preferred embodiment of the invention, Ar⁴ is an aromatic ring system. Preferred aromatic ring systems Ar⁴ are selected, identically or differently on each occurrence, from the group consisting of phenyl, 1- or 2-naphthyl, ortho-, meta- or para-biphenyl, 2-fluorenyl or 2-spirobifluorenyl, each of which may be substituted by one or more radicals R⁴.

Preferred radicals R⁴ are selected, identically or differently on each occurrence, from the group consisting of H, D, F, CN, straight-chain alkyl groups having 1 to 10 C atoms or branched alkyl groups having 3 to 10 C atoms.

Further preferred fluorescent dopants are the compounds of the following formula (340).

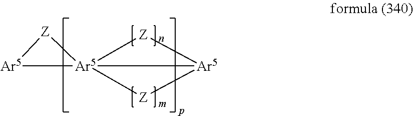

where R² has the above-mentioned meaning and the following applies to the other symbols and indices used:
Ar⁵ is on each occurrence, identically or differently, an aryl or heteroaryl group having 5 to 30 aromatic ring atoms, which may be substituted by one or more radicals R², with the proviso that at least one group Ar⁵ stands for a condensed aryl or heteroaryl group having 10 to 30 aromatic ring atoms;
Z is selected on each occurrence, identically or differently, from the group consisting of BR⁴, C(R⁴)₂, Si(R⁴)₂, C═O, C═NR⁴, C═C(R⁴)₂, O, S, S═O, SO₂, NR⁴, PR⁴ and P(═O) R⁴;
m, n is 0 or 1, with the proviso that m+n=1;
p is 1, 2 or 3;
   in each case two groups Ar⁵ and Z together form a five-membered ring or a six-membered ring, preferably in each case a five-membered ring.

In a preferred embodiment of the invention, the sum of all n-electrons in the groups Ar⁵ is at least 28 if p=1, and is at least 34 if p=2, and is at least 40 if p=3.

In a preferred embodiment of the invention, at least one group Ar⁵ stands for a condensed aryl group having 10 to 18 C atoms, in particular selected from the group consisting of naphthalene, phenanthrene, anthracene, pyrene, fluoranthene, naphthacene, chrysene, benzanthracene, benzophenanthrene and triphenylene and the other two groups Ar⁵ stand, identically or differently on each occurrence, for an aryl group having 6 having 18 C atoms, preferably, identically or differently on each occurrence, for phenyl or naphthyl.

In a further preferred embodiment of the invention, Z is selected, identically or differently on each occurrence, from the group consisting of C(R⁴)₂, C═O, NR⁴, O and S, particularly preferably, identically or differently on each occurrence, C(R⁴)₂ or NR⁴, very particularly preferably C(R⁴)₂.

Suitable fluorescent dopants are furthermore the structures depicted below, and the structures disclosed in JP 06/001973, WO 2004/047499, WO 2006/098080, WO 2007/065678, US 2005/0260442 and WO 2004/092111.

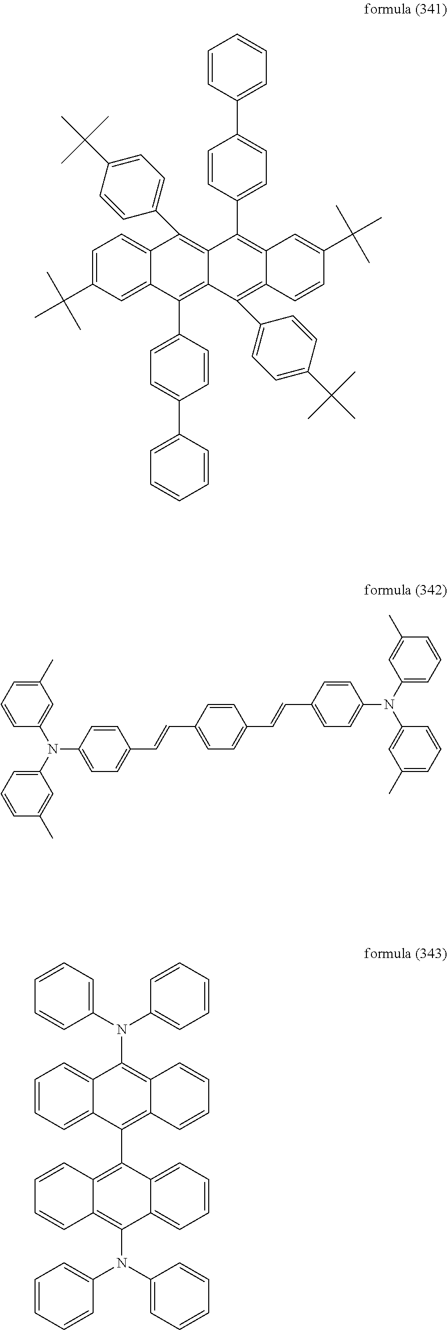

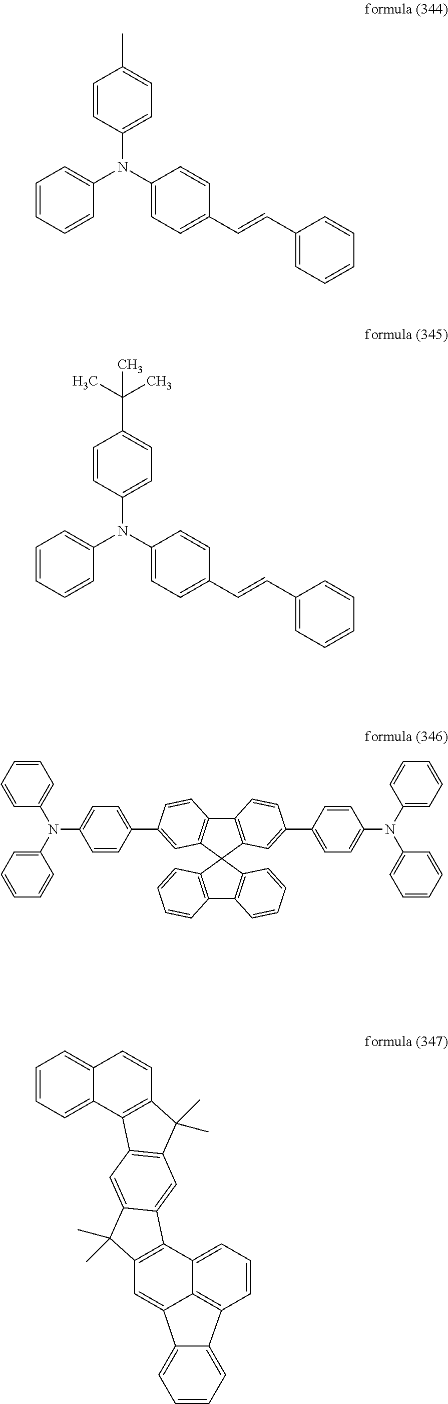

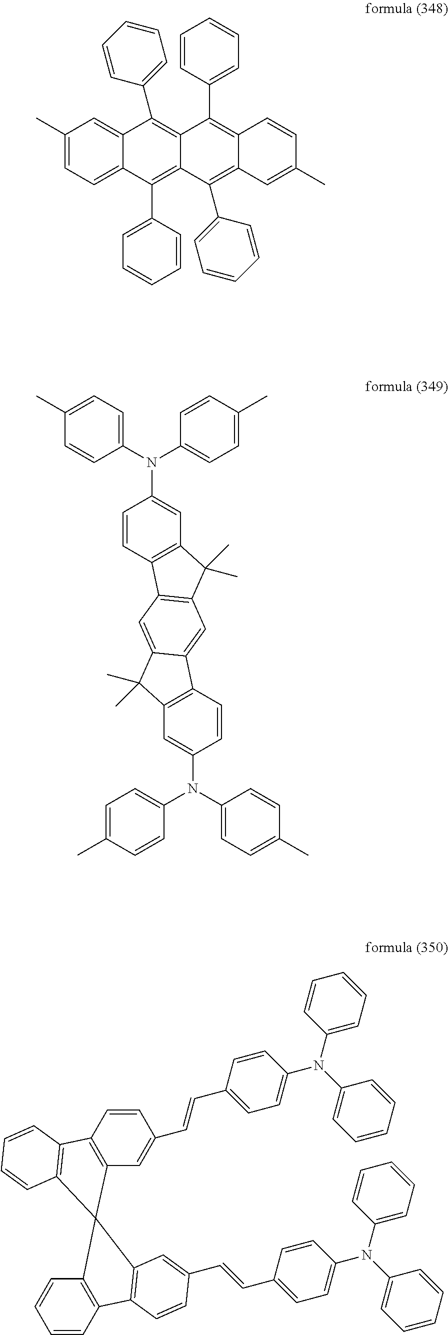

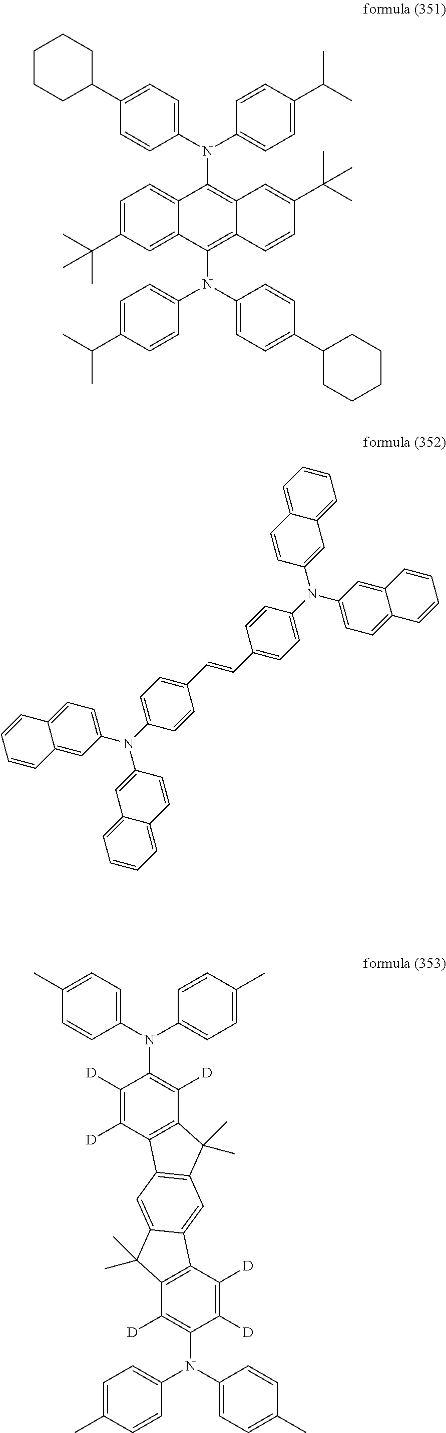

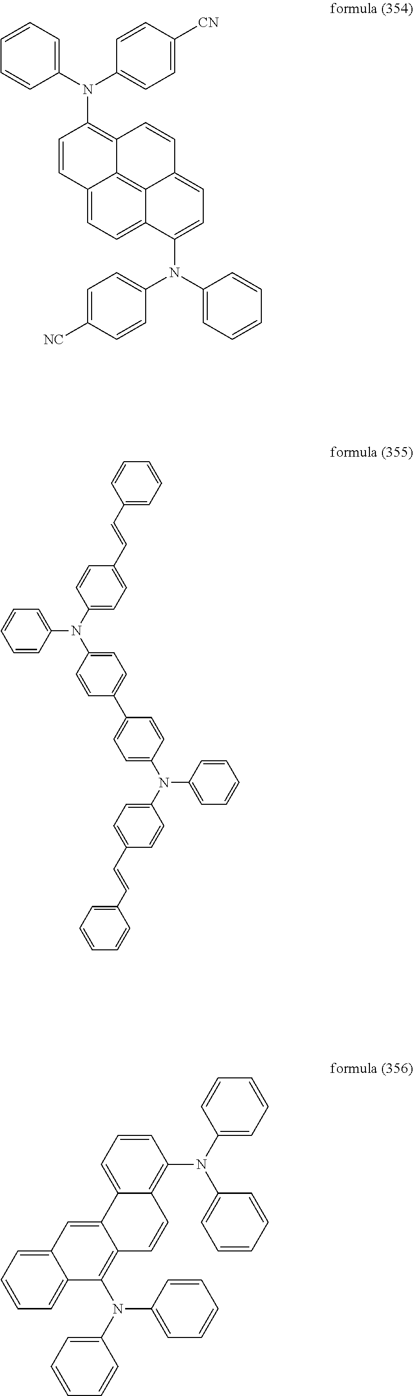 [SMILES: N#Cc1ccc(N(c2ccccc2)c2ccc3ccc4c(N(c5ccccc5)c5ccc(C#N)cc5)ccc5ccc2c3c54)cc1 ; C(=C\c1ccc(N(c2ccccc2)c2ccc(-c3ccc(N(c4ccccc4)c4ccc(/C=C/c5ccccc5)cc4)cc3)cc2)cc1)/c1ccccc1 ; c1ccc(N(c2ccccc2)c2cccc3c2ccc2c(N(c4ccccc4)c4ccccc4)c4ccccc4cc23)cc1]

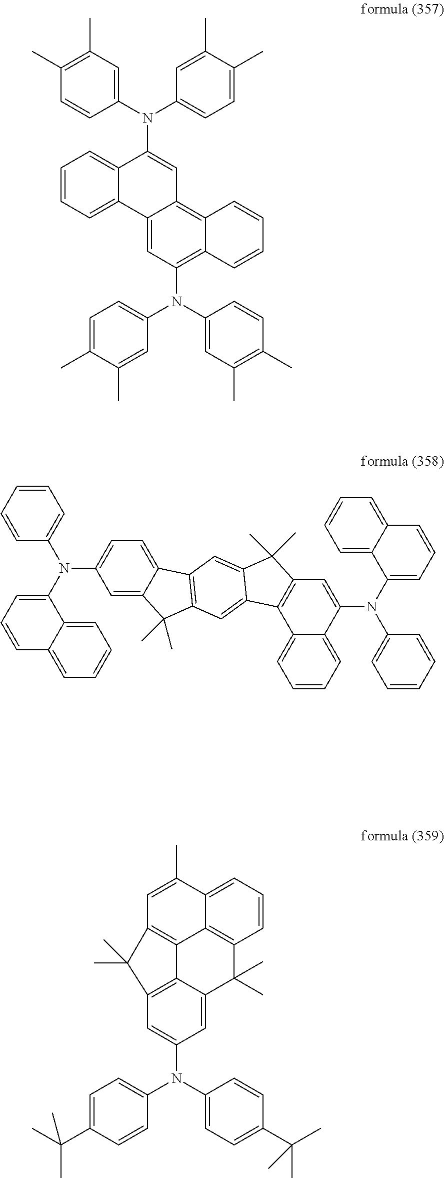

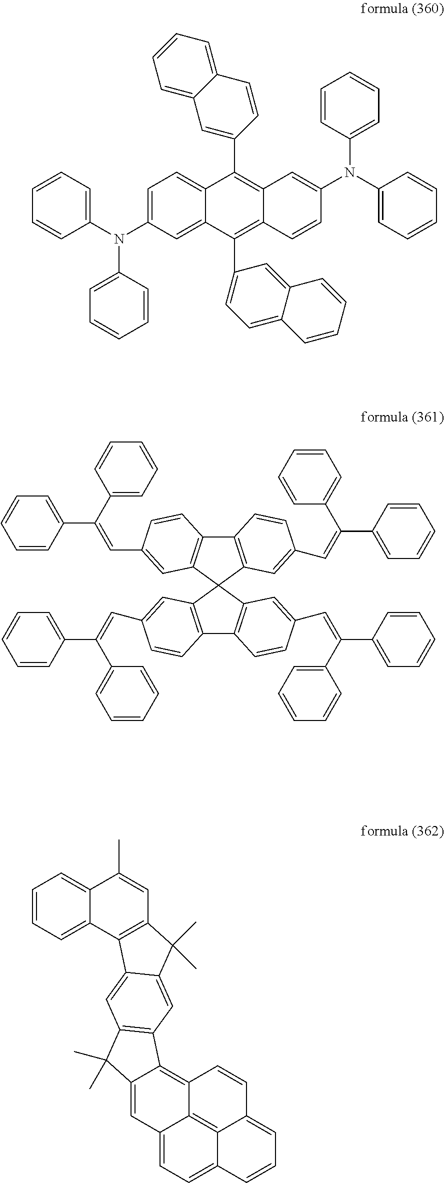

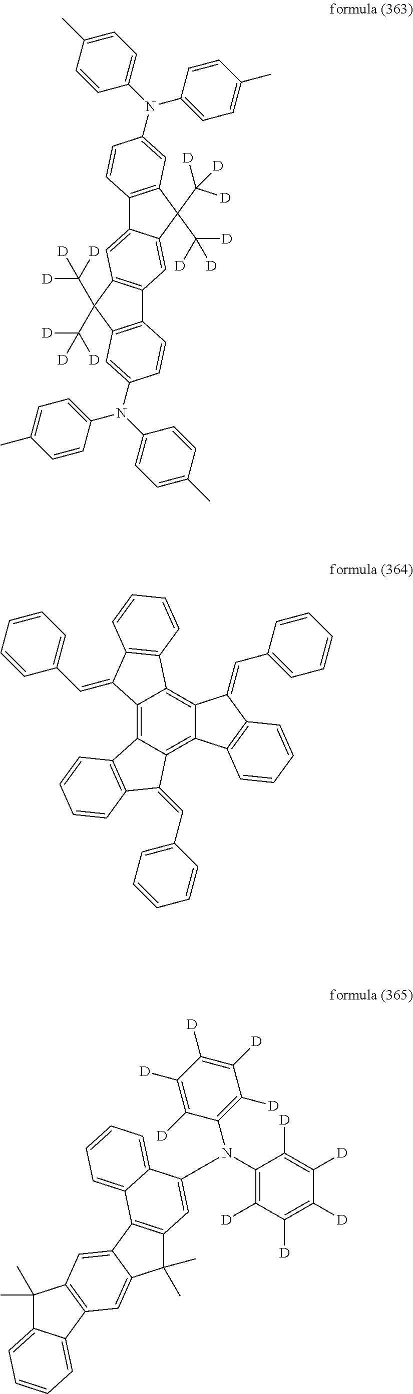

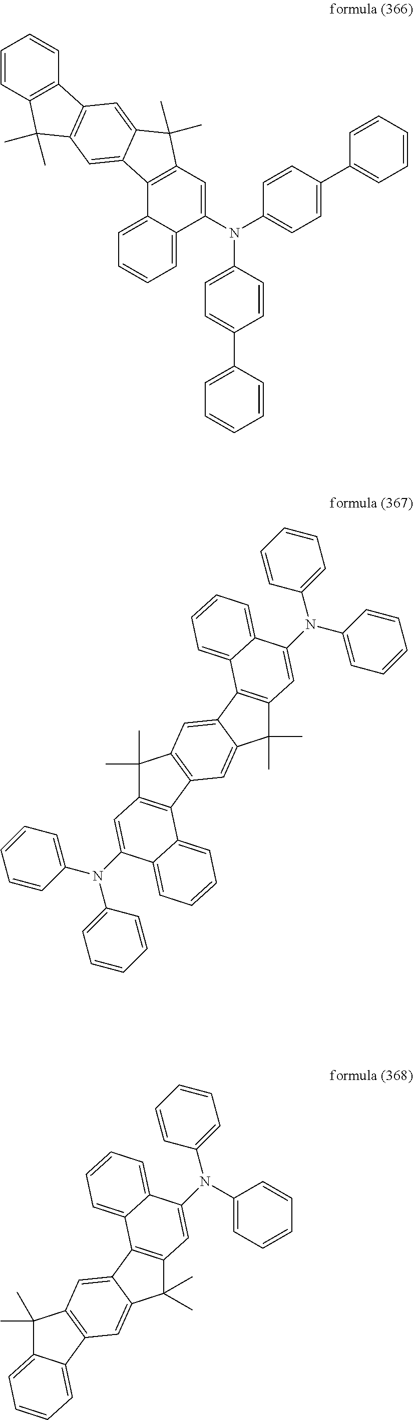 [SMILES: CC1(C)c2ccccc2-c2cc3c(cc21)-c1c(cc(N(c2ccc(-c4ccccc4)cc2)c2ccc(-c4ccccc4)cc2)c2ccccc12)C3(C)C ; CC1(C)c2cc3c(cc2-c2c1cc(N(c1ccccc1)c1ccccc1)c1ccccc21)C(C)(C)c1cc(N(c2ccccc2)c2ccccc2)c2ccccc2c1-3 ; CC1(C)c2ccccc2-c2cc3c(cc21)-c1c(cc(N(c2ccccc2)c2ccccc2)c2ccccc12)C3(C)C]

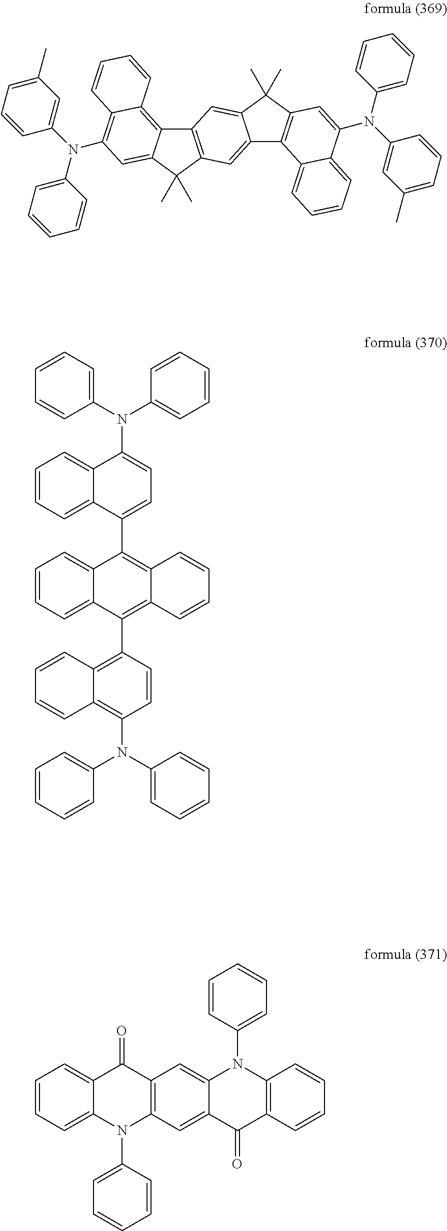 [SMILES: Cc1cccc(N(c2ccccc2)c2cc3c(c4ccccc24)-c2cc4c(cc2C3(C)C)-c2c(cc(N(c3ccccc3)c3cccc(C)c3)c3ccccc23)C4(C)C)c1 ; c1ccc(N(c2ccccc2)c2ccc(-c3c4ccccc4c(-c4ccc(N(c5ccccc5)c5ccccc5)c5ccccc45)c4ccccc34)c3ccccc23)cc1 ; O=c1c2ccccc2n(-c2ccccc2)c2cc3c(=O)c4ccccc4n(-c4ccccc4)c3cc12]

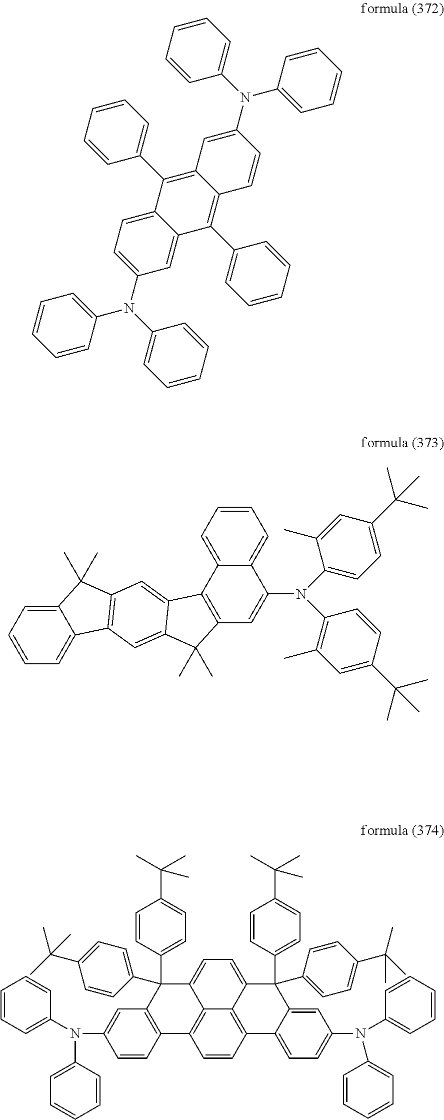 [SMILES: c1ccc(-c2c3ccc(N(c4ccccc4)c4ccccc4)cc3c(-c3ccccc3)c3ccc(N(c4ccccc4)c4ccccc4)cc23)cc1 ; Cc1cc(C(C)(C)C)ccc1N(c1ccc(C(C)(C)C)cc1C)c1cc2c(c3ccccc13)-c1cc3c(cc1C2(C)C)-c1ccccc1C3(C)C ; CC(C)(C)c1ccc(C2(c3ccc(C(C)(C)C)cc3)c3cc(N(c4ccccc4)c4ccccc4)ccc3-c3ccc4c5c(ccc2c35)C(c2ccc(C(C)(C)C)cc2)(c2ccc(C(C)(C)C)cc2)c2cc(N(c3ccccc3)c3ccccc3)ccc2-4)cc1]

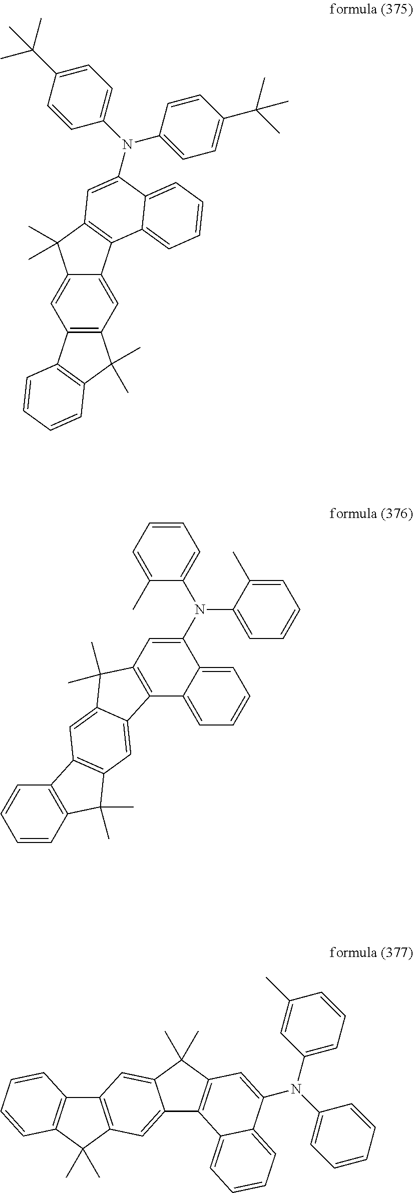 [SMILES: CC(C)(C)c1ccc(N(c2ccc(C(C)(C)C)cc2)c2cc3c(c4ccccc24)-c2cc4c(cc2C3(C)C)-c2ccccc2C4(C)C)cc1 ; Cc1ccccc1N(c1ccccc1C)c1cc2c(c3ccccc13)-c1cc3c(cc1C2(C)C)-c1ccccc1C3(C)C ; Cc1cccc(N(c2ccccc2)c2cc3c(c4ccccc24)-c2cc4c(cc2C3(C)C)-c2ccccc2C4(C)C)c1]

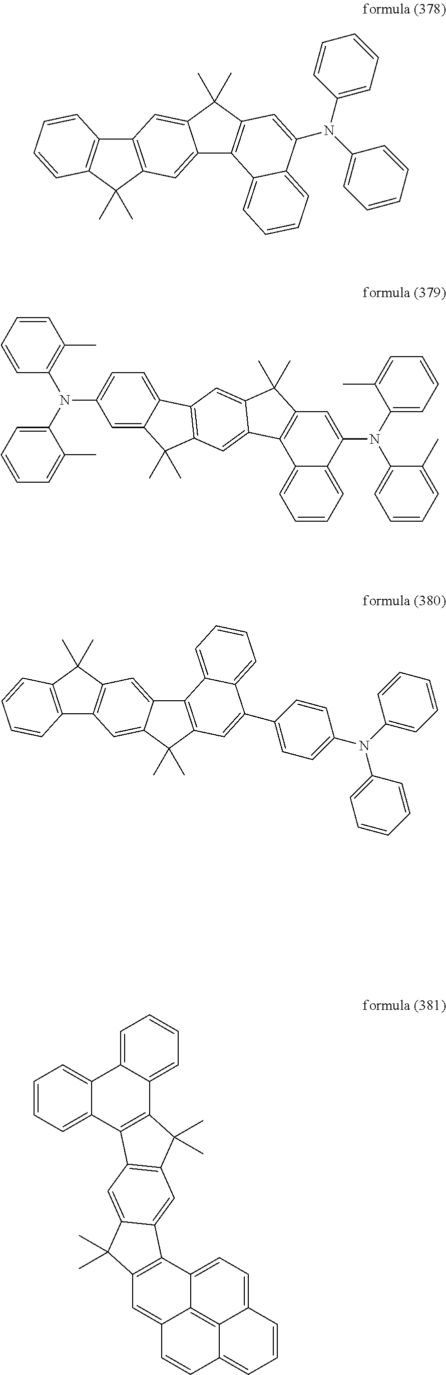

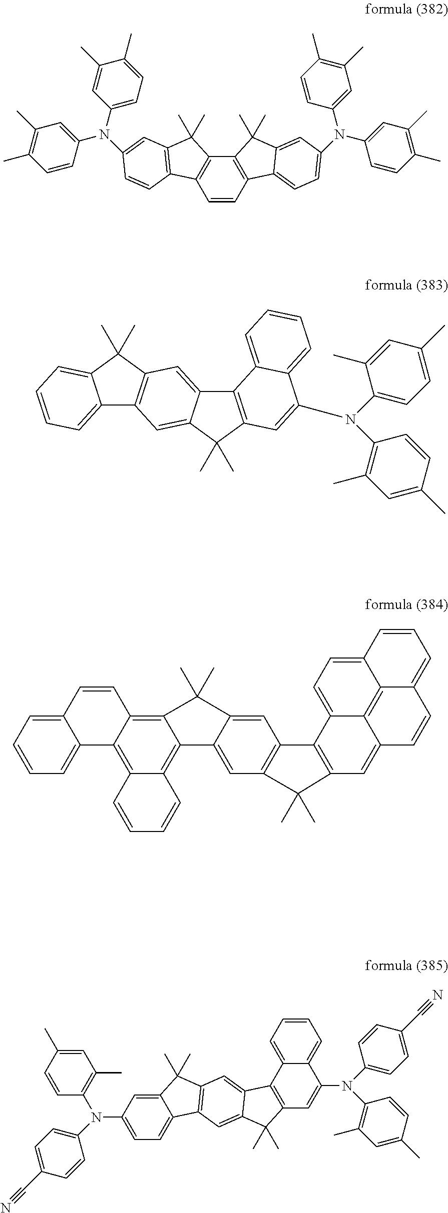

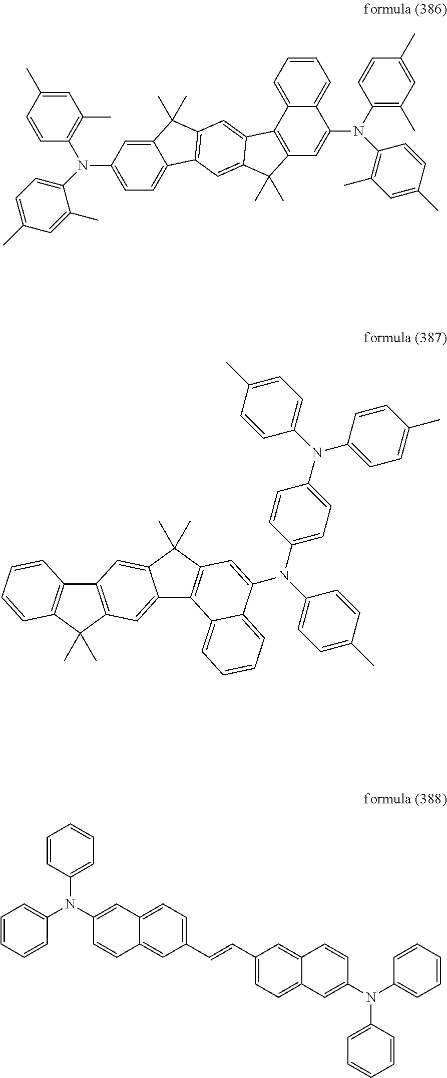

The compositions according to the invention are characterised in that the triplet level of the sensitiser T₁(S) is greater than the triplet level of the emitter T₁(E).

In an embodiment, the compositions according to the invention are characterised in that the singlet level of the emitter S₁(E) is higher than the singlet level of the sensitiser S₁(S) (FIG. 1).

In a further preferred embodiment, the compositions according to the invention are characterised in that the singlet level of the emitter S₁(E) is lower than the singlet level of the sensitiser S₁(S) (FIG. 2).

The ISC rate of the sensitiser here should be higher than the emission rate of the sensitiser from S₁(S). The ISC rate of an organic compound can be determined by means of “Zeeman phosphorescence microwave double resonance (PMDR) Spectroscopy, as Zinsli et. al. in Chem. Phys. Lett. Vol 34, 403(1975) have described—The ISC rate of quinoxaline has also been determined therein. The very high ISC rate of naphthyridine, phthalazine and quinoxaline has already been confirmed by Boldridge et al. (J. Phys. Chem. 86, 1976, 1982) and by Komorowski et al. (J. Photochem. 30, 141, 1985).

The compositions according to the invention are characterised in that the quantum yield of the phosphorescence of the sensitiser at 20° C. or higher temperatures is very low, preferably not more than 2%, very preferably not more than 1%, very particularly preferably not more than 0.2%. The sensitiser especially preferably exhibits neither fluorescence nor phosphorescence at 20° C.

As already explained above, the compositions according to the invention are suitable for UpC. The present invention therefore furthermore relates to the use of the composition according to the invention comprising at least one compound of the general formula (1) or of the general formula (2) and at least one fluorescent emitter for UpC, in particular for UpC in electroluminescent devices.

The compositions according to the invention are employed here in the emission layer. The present invention therefore also relates to an emission layer comprising the compositions according to the invention.

The present technical teaching can be generalised further to all up-conversion systems or compositions which can be employed for the purpose of up-conversion in order to develop electroluminescent devices which emit light in the blue region of the spectrum or UV radiation.

The present invention therefore also relates to the use of a composition for up-conversion in electroluminescent devices for the generation of light or radiation in the UV region.

The devices are preferably organic electroluminescent devices.

In the present application, blue light should preferably be taken to mean light having a wavelength in the range from 380 and 490 nm.

UV radiation in the sense of the present invention is preferably radiation having a wavelength in the range from 200 and 380 nm. Especial preference is given to the emission of UV-A radiation (315 to 380 nm) and/or of UV-B radiation (280 to 315 nm).

The present invention furthermore relates to electroluminescent device comprising one or more compositions for up-conversion for the generation of light or radiation in the UV region.

The electroluminescent device is preferably an organic electroluminescent device. The device preferably emits in the above-mentioned preferred wavelength ranges.

The present invention furthermore relates to optical and/or electronic devices for up-conversion comprising at least one composition according to the invention.

The devices here can be selected from the group consisting of organic electroluminescent devices, such as, for example, organic light-emitting diodes (OLED), organic light-emitting transistors, organic light-emitting electrochemical cells, organic light-emitting electrochemical transistors, or an organic laser, where an OLED is particularly preferred.

The organic electroluminescent device comprises cathode, anode and at least one emitting layer. Apart from these layers, it may also comprise further layers, for example in each case one or more hole-injection layers, hole-transport layers, hole-blocking layers, electron-transport layers, electron-injection layers, exciton-blocking layers and/or charge-generation layers. It is likewise possible for interlayers, which have, for example, an exciton-blocking function, to be introduced between two emitting layers. However, it should be pointed out that each of these layers does not necessarily have to be present. A possible layer structure is, for example, the following: cathode/EML/interlayer/buffer layer/anode, where EML represents the emitting layer. The organic electroluminescent device here may comprise one emitting layer, or it may comprise a plurality of emitting layers.

In a further embodiment of the invention, the organic electroluminescent device according to the invention does not comprise a separate hole-injection layer and/or hole-transport layer and/or hole-blocking layer and/or electron-transport layer, i.e. the emitting layer is directly adjacent to the hole-injection layer or the anode, and/or the emitting layer is directly adjacent to the electron-transport layer or the electron-injection layer or the cathode, as described, for example, in WO 2005/053051.

Preference is furthermore given to an organic electroluminescent device, characterised in that one or more layers are coated by means of a sublimation process, in which the materials are vapour-deposited in vacuum sublimation units at an initial pressure of less than 10⁻⁵ mbar, preferably less than 10⁻⁶ mbar. However, it is also possible for the initial pressure to be even lower, for example less than 10⁻⁷ mbar.

Preference is likewise given to an organic electroluminescent device, characterised in that one or more layers are coated by means of the OVPD (organic vapour phase deposition) process or with the aid of carrier-gas sublimation, in which the materials are applied at a pressure between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, offset printing, LITI (light induced thermal imaging, thermal transfer printing), inkjet printing or nozzle printing. Soluble compounds, which are obtained, for example, by suitable substitution, are necessary for this purpose. These processes are also suitable, in particular, for oligomers, dendrimers and polymers.

A further embodiment of the present invention relates to formulations comprising one or more of the compositions according to the invention and one or more solvents. The formulation is highly suitable for the production of layers from solution.

Suitable and preferred solvents are, for example, toluene, anisole, xylenes, methyl benzoate, dimethylanisoles, trimethylbenzenes, tetralin, veratrols, tetrahydrofuran, cyclohexanone, chlorobenzene or dichlorobenzenes and mixtures thereof.

These processes are generally known to the person skilled in the art and can be applied by him without inventive step to organic electroluminescent devices comprising the compounds according to the invention.

The organic electroluminescent device according to the invention can be used, for example, in displays or for lighting purposes, but also for medical or cosmetic applications.

The compositions according to the invention are suitable for use in light-emitting devices. These compounds can thus be employed in a very versatile manner. Some of the main areas of application here are display or lighting technologies. It is furthermore particularly advantageous to employ the compositions and devices comprising the compositions in the area of phototherapy.

The present invention therefore furthermore relates to the use of the compositions according to the invention and devices comprising the compositions for the treatment, prophylaxis and diagnosis of diseases. The present invention still furthermore relates to the use, of the compositions according to the invention and devices comprising the compositions in cosmetics.

The present invention furthermore relates to the compositions according to the invention and devices comprising the compositions for the production of equipment, i.e. irradiation equipment, for the therapy, prophylaxis and/or diagnosis of therapeutic diseases.

The present invention furthermore relates to devices comprising the compositions according to the invention for use for the treatment of the skin using phototherapy.

The present invention also relates to the use of the device comprising the compositions according to the invention in cosmetics.

Phototherapy or light therapy is used in many medical and/or cosmetic areas. The compositions according to the invention and the devices comprising the compositions can therefore be employed for the therapy and/or prophylaxis and/or diagnosis of all diseases and/or in cosmetic applications for which the person skilled in the art considers the use of phototherapy. Besides simple irradiation, the term phototherapy also includes photodynamic therapy (PDT) as well as disinfection, sterilisation and preservation in general. It is not only humans or animals that can be treated by means of phototherapy or light therapy, but also any other type of living or non-living materials. These include, for example, fungi, bacteria, microbes, viruses, eukaryotes, prokaryotes, foods, drinks, water and drinking water. Containers for keeping food or other articles fresh can also be provided with the devices according to the invention.

The term phototherapy also includes any type of combination of light therapy and other types of therapy, such as, for example, treatment with active compounds. Many light therapies have the aim of irradiating or treating exterior parts of an object, such as the skin of humans and animals, wounds, mucous membranes, the eye, hair, nails, the nail bed, gums and the tongue. In addition, the treatment or irradiation according to the invention can also be carried out inside an object in order, for example, to treat internal organs (heart, lung, etc.) or blood vessels or the breast.

The therapeutic and/or cosmetic areas of application according to the invention are preferably selected from the group of skin diseases and skin-associated diseases or changes or conditions, such as, for example, psoriasis, skin ageing, skin wrinkling, skin rejuvenation, enlarged skin pores, cellulite, oily/greasy skin, folliculitis, actinic keratosis, precancerous actinic keratosis, skin lesions, sun-damaged and sun-stressed skin, crows' feet, skin ulcers, acne, acne rosacea, scars caused by acne, acne bacteria, photomodulation of greasy/oily sebaceous glands and their surrounding tissue, jaundice, jaundice of the newborn, vitiligo, skin cancer, skin tumours, Crigler-Najjar, dermatitis, atopic dermatitis, diabetic skin ulcers, and desensitisation of the skin.

Particular preference is given for the purposes of the invention to the treatment and/or prophylaxis of psoriasis, acne, cellulite, skin wrinkling, skin ageing, jaundice and vitiligo.

Further areas of application according to the invention for the compositions and/or devices comprising the compositions according to the invention are selected from the group of inflammatory diseases, rheumatoid arthritis, pain therapy, treatment of wounds, neurological diseases and conditions, oedema, Paget's disease, primary and metastasising tumours, connective-tissue diseases or changes, changes in the collagen, fibroblasts and cell level originating from fibroblasts in tissues of mammals, irradiation of the retina, neovascular and hypertrophic diseases, allergic reactions, irradiation of the respiratory tract, sweating, ocular neovascular diseases, viral infections, particularly infections caused by herpes simplex or HPV (human papillomaviruses) for the treatment of warts and genital warts.

Particular preference is given for the purposes of the invention to the treatment and/or prophylaxis of rheumatoid arthritis, viral infections and pain.

Further areas of application according to the invention for the compositions and/or devices comprising the compositions according to the invention are selected from winter depression, sleeping sickness, irradiation for improving the mood, the reduction in pain particularly muscular pain caused by, for example, tension or joint pain, elimination of the stiffness of joints and the whitening of the teeth (bleaching).

Further areas of application according to the invention for the compositions and/or devices comprising the compositions according to the invention are selected from the group of disinfections. The compositions according to the invention and/or the devices comprising the compositions according to the invention can be used for the treatment of any type of objects (non-living materials) or subjects (living materials such as, for example, humans and animals) for the purposes of disinfection, sterilisation or preservation. This includes, for example, the disinfection of wounds, the reduction in bacteria, the disinfection of surgical instruments or other articles, the disinfection or preservation of foods, of liquids, in particular water, drinking water and other drinks, the disinfection of mucous membranes and gums and teeth. Disinfection here is taken to mean the reduction in the living microbiological causative agents of undesired effects, such as bacteria and germs.

For the purposes of the phototherapy mentioned above, devices comprising the compounds according to the invention preferably emit light having a wavelength between 280 and 1000 nm, particularly preferably between 290 and 800 nm and especially preferably between 380 and 600 nm.

The compositions and/or devices comprising the compositions according to the invention are particularly advantageous owing to the fact that UV emission is also possible by means of UpC. This is important for certain areas of application and is not yet possible by means of devices from the prior art. Thus, for example, psoriasis is treated by irradiation with radiation of wavelength around 311 nm.

In a particularly preferred embodiment of the present invention, the compositions according to the invention are employed in an organic light-emitting diode (OLED) or an organic light-emitting electrochemical cell (OLEC) for the purposes of phototherapy. Both the OLED and the OLEC can have a planar or fibre-like structure having any desired cross section (for example round, oval, polygonal, square) with a single- or multilayered structure. These OLECs and/or OLEDs can be installed in other devices which comprise further mechanical, adhesive and/or electronic elements (for example battery and/or control unit for adjustment of the irradiation times, intensities and wavelengths). These devices comprising the OLECs and/or OLEDs according to the invention are preferably selected from the group comprising plasters, pads, tapes, bandages, sleeves, blankets, hoods, sleeping bags, textiles and stents.

The use of the said devices for the said therapeutic and/or cosmetic purpose is particularly advantageous compared with the prior art, since homogeneous irradiation with low irradiation intensity is possible at virtually any site and at any time of day with the aid of the devices according to the invention using the OLEDs and/or OLECs. The irradiation can be carried out as an inpatient, as an outpatient and/or by the patient themselves, i.e. without initiation by medical or cosmetic specialists. Thus, for example, plasters can be worn under clothing, so that irradiation is also possible during working hours, in leisure time or during sleep. Complex inpatient/outpatient treatments can in many cases be avoided or their frequency reduced. The devices according to the invention may be intended for re-use or be disposable articles, which can be disposed of after use once, twice or three times.

Further advantages over the prior art are, for example, lower evolution of heat and emotional aspects. Thus, newborn being treated owing to jaundice typically have to be irradiated blindfolded in an incubator without physical contact with the parents, which represents an emotional stress situation for parents and newborn. With the aid of a blanket according to the invention comprising the OLEDs and/or OLECs according to the invention, the emotional stress can be reduced significantly. In addition, better temperature control of the child is possible due to reduced heat production of the devices according to the invention compared with conventional irradiation equipment.

The compositions according to the invention and/or devices comprising the compositions according to the invention, in particular organic electroluminescent devices, are distinguished by the following surprising advantages over the prior art:
1. The organic sensitisers, the compositions and devices comprising same are significantly more stable, especially to atmospheric oxygen and other environmental influences, than metal complexes from the prior art.
2. The organic sensitisers, compositions and formulations comprising same are very simple to prepare and are also particularly suitable for mass production.
3. The devices according to the invention provide higher efficiencies than all previous electroluminescent devices for UpC.
4. The devices according to the invention enable efficient utilisation of triplet excitons without compounds containing a heavy-metal atom being used. Without the use of heavy-metal atoms, triplet excitons normally cannot be utilised (FIGS. 3 and 4).
5. The devices according to the invention enable the achievement of UV emissions using standard electroluminescent devices.
6. The operating voltage of the devices according to the invention is very low.
7. The devices according to the invention exhibit no residual emission in the long-wave region, which is frequently evident in compositions in accordance with the prior art.
8. Many compositions according to the invention can be processed from solution. Thus, layers can be formed in a simple manner and produced inexpensively.

The said advantages are not accompanied by an impairment in the other electronic properties.

Even without further comments, it is assumed that a person skilled in the art will be able to utilise the above description in the broadest scope.

It should be pointed out that variations of the embodiments described in the present invention fall within the scope of this invention. Each feature disclosed in the present invention can, unless explicitly excluded, be replaced by alternative features which serve the same, an equivalent or a similar purpose. Thus, each feature disclosed in the present invention should, unless stated otherwise, be regarded as an example of a generic series or as an equivalent or similar feature.

All features of the present invention can be combined with one another in any way, unless certain features and/or steps are mutually exclusive. This applies, in particular, to preferred features of the present invention. Equally, features of non-essential combinations can be used separately (and not in combination).

It should furthermore be pointed out that many of the features, and in particular those of the preferred embodiments of the present invention, should be regarded as inventive themselves and not merely as part of the embodiments of the present invention. Independent protection may be granted for these features in addition or as an alternative to each invention claimed at present. The teaching regarding technical action disclosed with the present invention can be abstracted and combined with other examples. The invention is explained in greater detail by the following examples and figure, without wishing it to be restricted thereby.

### BRIEF DESCRIPTION OF THE FIGURES

FIG. 1: Simplified Jablonski diagram for illustration of up-conversion by means of TTA (triplet-triplet annihilation) with optical excitation. The sensitiser (I) is excited from the ground state S₀ into the excited singlet state S₁ by means of the energy Eᵢₙ (in the form of photon). Intersystem crossing (ISC), i.e. the transition into the first excited triplet state T₁ with spin inversion, occurs. The energy from T₁ of the sensitiser is then transferred to the T₁ level of the acceptor (II) (TTET—triplet-triplet energy transfer), with phosphorescence hν₁ from T₁ of the sensitiser being possible as a competing process. Owing to the purely organic character of the sensitisers of this invention, this competing process is greatly suppressed here compared with heavy-metal-containing sensitisers in accordance with the prior art. Finally, a bimolecular impact between two acceptors, both of which are in the excited T₁ state, results in the first acceptor being converted into the excited Sn state and the other acceptor being converted into the electronic ground state S₀. After relaxation (IC—internal conversion) from Sₙ to S₁, the emission hνₒᵤₜ of the acceptor occurs from the S₁ state

FIG. 2: An embodiment with optical excitation, where the first excited singlet level of the emitter S₁(E) is lower than that of the sensitiser S₁(S).

FIG. 3: An embodiment with electrical excitation, where the first excited singlet level of the emitter S₁(E) is higher than that of the sensitiser S₁(S). The energy Eᵢₙ represents the energy of the electron/hole pair, where the electrons have been injected from the cathode and the holes from the anode. The electron/hole pair recombines on the sensitiser. The excited states S₁ and T₁ form. The further process corresponds to that in FIG. 1.

FIG. 4: An embodiment with electrical excitation, where the first excited singlet level of the emitter S₁(E) is lower than that of the sensitiser S₁(S).

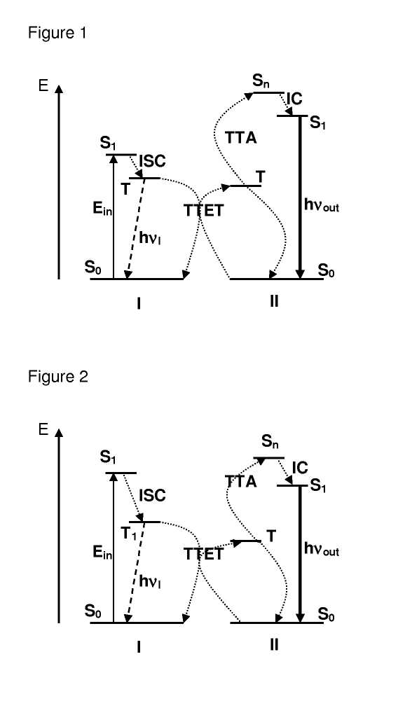

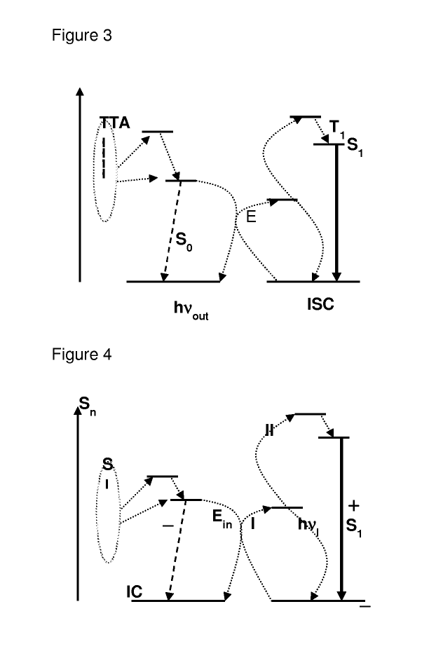

### EXAMPLES

### Example 1

### Materials and Synthesis

Polymer H1, which contains monomers (M1-M4) in the mole percentages below, is prepared by SUZUKI coupling in accordance with WO 2003/048225. H1 is used as sensitiser according to the invention.

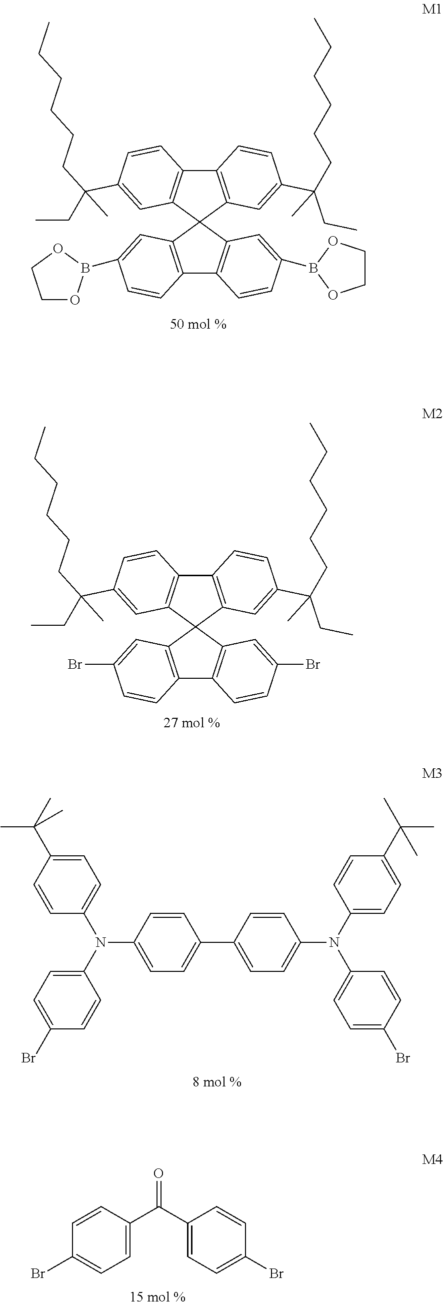

H2 is prepared in accordance with WO 2004/093207.

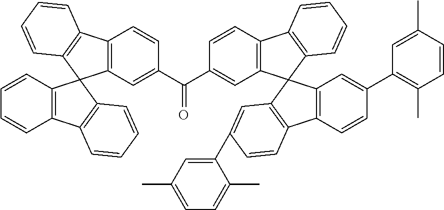 [SMILES: Cc1ccc(C)c(-c2ccc3c(c2)C2(c4ccccc4-c4ccc(C(=O)c5ccc6c(c5)C5(c7ccccc7-c7ccccc75)c5ccccc5-6)cc42)c2cc(-c4cc(C)ccc4C)ccc2-3)c1]

H1 and H2 are used as sensitisers. Their PL spectra (photoluminescence) exhibit a weak signal in the case of H1 and only noise for H2 for excitation at 325 nm. This is evidence of a high intersystem crossing rate of the two sensitisers. Emitter1 is prepared in accordance with WO 2008/006449 and Emitter2 in accordance with DE 102008035413.

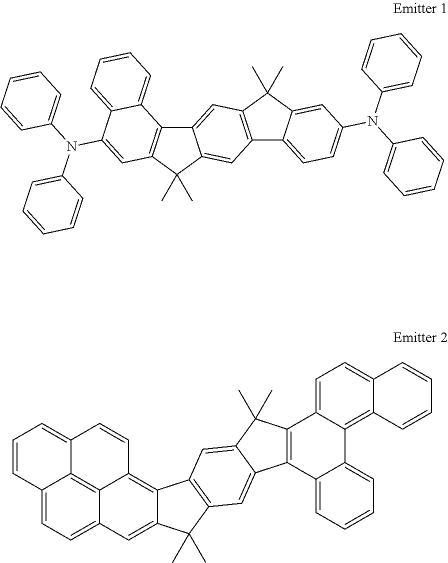

The reference materials employed are organic sensitisers which are known as sensitisers for UpC in photoluminescence (In accordance with J. Phys. Chem. A, Vol. 113, 2009 5913 and RSC Advances, 2011, 1, 937):

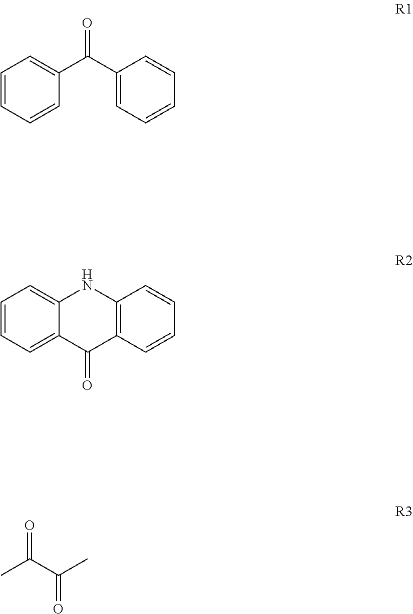

R3 is unsuitable for electroluminescent devices owing to its fluid character.

### Example 2

### Quantum-Chemical Simulations of the Materials

The HOMO (highest occupied molecular orbital) and LUMO (lowest unoccupied molecular orbital) positions and the triplet/singlet level of the organic organic compounds are determined via quantum-chemical calculations. To this end, the “Gaussian03W” program package (Gaussian Inc.) is used. In order to calculate organic substances without metals, firstly a geometry optimisation is carried out with the aid of a semi-empirical “Ground State/Semi-empirical/Default Spin/AM1” method (Charge 0/Spin Singlet). An energy calculation is subsequently carried out on the basis of the optimised geometry. In this, the “TD-SCF/DFT/Default Spin/B3PW91” (time dependent−self consistent field/density functional theory) method with the “6-31 G(d)” base set (Charge 0/Spin Singlet) is used. The most important results are HOMO/LUMO levels and energies for the triplet and singlet excited states. The first excited triplet and singlet states, T1 and S1, are the most important here. The energy calculation gives the HOMO HEh or LUMO LEh in hartree units. The HOMO and LUMO values in electron volts (eV) are determined therefrom as follows, where these relationships arise from the calibration with reference to cyclic voltammetry measurements (CV):

HOMO(eV)=((HEh*27.212)−0.9899)/1.1206

LUMO(eV)=((LEh*27.212)−2.0041)/1.385

These values are to be regarded for the purposes of this application as the energetic position of the HOMO level or LUMO level of the materials. As an example, an HOMO of −0.20435 hartrees and an LUMO of −0.06350 hartrees are obtained from the calculation for compound H2 (Table 1), which [lacuna] a calibrated HOMO of −5.85 eV, a calibrated LUMO of −2.69 eV.

TABLE 1

| | Homo corr. | Lumo corr. | Triplet T1 | Singlet S1 |
|---|---|---|---|---|
| Material | [eV] | [eV] | [eV] | [eV] |
| H2 | −5.85 | −2.69 | 2.70 | 3.35 |
| Emitter1 | −5.12 | −2.60 | 1.98 | 2.76 |
| Emitter2 | −5.37 | −2.86 | 1.81 | 2.81 |

For polymers, in particular conjugated polymers, the calculations are restricted to trimers, i.e. for a polymer containing monomers M1 and M2 the trimers M2−M1−M2 and/or M1−M2−M1 are calculated, with polymerisable groups being removed. Furthermore, long alkyl chains are reduced to a short chain. This will be illustrated by way of example in the following description with reference to polymer H1. The good agreement between CV measurements and simulations of polymers is disclosed in WO 2008/011953 A1.

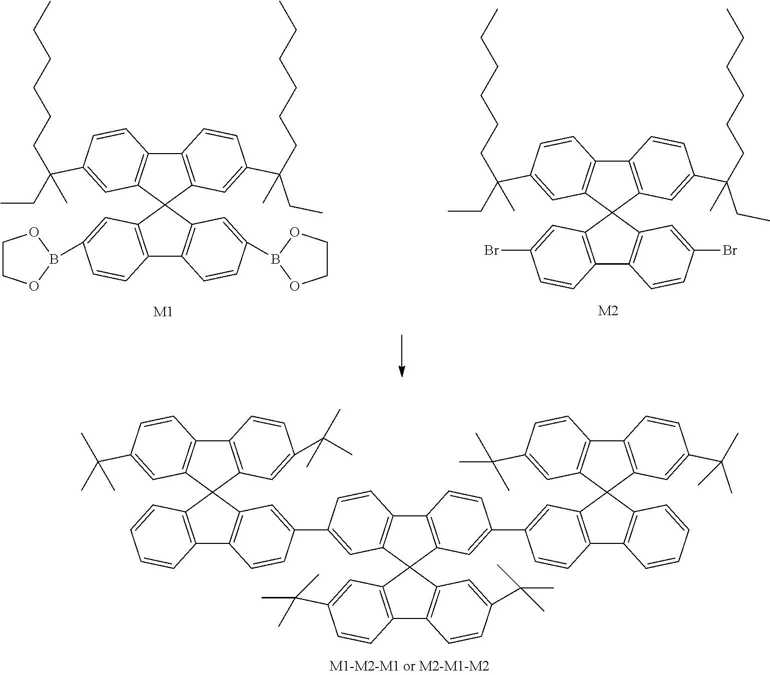

These values are to be regarded for the purposes of this application as the energetic position of the HOMO level or LUMO level of the materials. As an example, an HOMO of −0.19301 hartrees and an LUMO of −0.05377 hartrees are obtained by means of simulation for polymer P1 (M1−M2−M1 in Table 2), which corresponds to a calibrated HOMO of −5.57 eV, a calibrated LUMO of −2.50 eV.

TABLE 2
Energy level of polymer H1

| | Homo corr. | Lumo corr. | Singlet S1 | Triplet T1 |
|---|---|---|---|---|
| | [eV] | [eV] | [eV] | [eV] |
| M1-M2-M1 | −5.57 | −2.50 | 3.04 | 2.48 |
| M1-M3-M1 | −5.03 | −2.32 | 3.02 | 2.47 |
| M1-M4-M1 | −5.86 | −2.82 | 3.26 | 2.60 |

It is evident from the results from Table 1 and 2 that H1 and H2 have a T1 and S1 level which is higher than that of Emitter1 and Emitter2.

### Example 3

### Solutions and Compositions Comprising Sensitisers and Emitters 1 or 2

Solutions as summarised as in Table 3 are prepared as follows: firstly, the sensitisers and the emitters are dissolved in the concentration indicated in 10 ml of chlorobenzene and stirred until the solution is clear. The solution is filtered using a Millipore Millex LS, hydrophobic PTFE 5.0 μm filter.

TABLE 3

| | | Ratio | |
|---|---|---|---|
| | | (based on | |
| | Composition | weight) | Concentration |
| Solution 1 | H1 + Emitter1 | 93%:7% | 12 mg/ml |
| Solution 2 | H1 + Emitter2 | 93%:7% | 12 mg/ml |
| Solution 3 | H2 + Emitter1 | 93%:7% | 24 mg/ml |
| Solution 4 | H2 + Emitter2 | 93%:7% | 24 mg/ml |
| Solution 5 | R1 + Emitter1 | 93%:7% | 24 mg/ml |
| Solution 6 | R2 + Emitter1 | 93%:7% | 24 mg/ml |

The solutions are used in order to coat the emitting layer of OLEDs. The corresponding solids composition can be obtained by evaporating the solvent of the solutions. This can be used for the preparation of further formulations.

### Example 4

### Production of the OLEDs

OLED1 to OLED6 having the typical layer sequence, ITO/PEDOT/interlayer/EML/cathode (ITO—indium tin oxide anode; EML—emission layer), are produced as follows using the corresponding solutions from Table 3, i.e. OLED1 is produced by means of solution 1, OLED2 by means of solution 2, etc.
1. Application of 80 nm of PEDOT (Baytron P Al 4083) to an ITO-coated glass substrate by spin coating. Subsequent drying by heating at 120° C. for 10 minutes.
2. Application of 20 nm of an interlayer by spin coating of a toluene solution of HIL-012 (Merck KGaA) (concentration 0.5% by weight) in a glove box.
3. Drying of the interlayer by heating at 180° C. for 1 h in a glove box.
4. Application of 80 nm of the emitting layer by spin coating of one of the solution from Table 3.
5. Drying of the device by heating at 180° C. for 10 min.
6. Application of a Ba/Al cathode by vapour deposition (3 nm+150 nm).
7. Encapsulation of the device.

Only techniques which are well known to the person skilled in the art are employed in the production of the devices.

OLED5 and OLED6 serve as comparative examples.

### Example 5

### Characterisation of the OLEDs

The OLEDs obtained in this way are characterised by standard methods which are well known to the person skilled in the art in the area. The following properties are measured here: UIL characteristics, electroluminescence spectrum, colour coordinates, efficiency and operating voltage. The results are summarised in Table 4, Where OLED5 and OLED6 serve as comparison in accordance with the prior art. In Table 4, U(100) stands for the voltage at 100 cd/m², and U(1000) stands for the voltage at 1000 cd/m². The data for the two OLEDs 5 and 6 cannot be determined since they have not exhibited any electroluminescence.

TABLE 4

| | Max. | U | U | | | Max. |
|---|---|---|---|---|---|---|
| | eff. | (1000) | (100) | CIEx @ | CIEy @ | EQE |
| | [cd/A] | [V] | [V] | 100 cd/m² | 100 cd/m² | % |
| OLED1 | 1.3 | 5.7 | 4.3 | 0.19 | 0.31 | 0.63 |
| OLED2 | 1.2 | 5.8 | 4.3 | 0.16 | 0.22 | 0.71 |
| OLED3 | 1.2 | 9.3 | 6.3 | 0.19 | 0.34 | 0.52 |
| OLED4 | 1.2 | 8.4 | 5.8 | 0.16 | 0.26 | 0.67 |
| OLED5 | — | — | — | — | — | — |
| OLED6 | — | — | — | — | — | — |

All sensitisers H1-H2 contain benzophenones or derivatives.

As Table 4 shows, surprisingly good OLEDs can be produced with the sensitisers and compositions according to the invention (OLED1, 2, 3, and 4). It should be taken into account here that the devices have not yet been optimised for electroluminescence. The person skilled in the art will be able to improve them further by means of routine experiments without inventive step using techniques which are well known to him.

Furthermore, the absolute PL efficiency (photoluminescence) of the emitting layer of OLED1 and OLED2 is measured. The efficiencies of both are less than 0.5%, which is even lower than the corresponding EQE. Sensitiser H2 does not exhibit a PL signal in the layer. In this connection, the mechanism of the devices according to the invention can best be explained by means of the TTA-UpC proposed. Comparative Examples OLED5 and 6 have not functioned.

### Example 6

### TTA-UpC by Means of Quinolxalines of the Formula (2)

Analogously to Examples 3, 4 and 5 above, compositions and OLEDs comprising sensitisers of the formula (2) can be prepared and characterised. For example, the two following compounds, H3 and H4, can be used for this purpose.

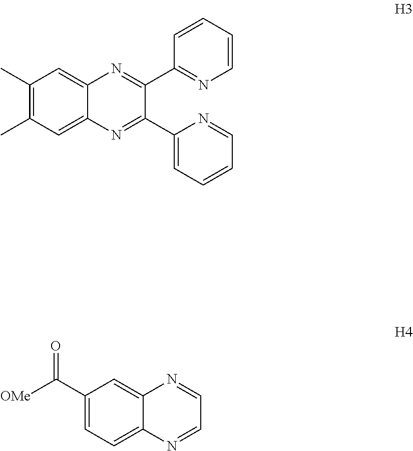 [SMILES: Cc1cc2nc(-c3ccccn3)c(-c3ccccn3)nc2cc1C ; COC(=O)c1ccc2nccnc2c1]

Both compounds are commercially available (Sigma Aldrich).

The energy levels of H3 and H4 are listed in Table 5 below.

TABLE 5

| | Homo corr. | Lumo corr. | Triplet T1 | Singlet S1 |
|---|---|---|---|---|
| Material | [eV] | [eV] | [eV] | [eV] |
| H3 | −6.51 | −2.81 | 2.70 | 3.44 |
| H4 | −7.16 | −3.24 | 2.71 | 3.38 |
| Emitter1 | −5.12 | −2.60 | 1.98 | 2.76 |

It is found that OLEDs comprising H3 or H4 as sensitiser (93 wt %) and Emitter1 as emitter (7 wt %) in the EML, are particularly advantageous, where H3 exhibits particularly good results. The electroluminescent devices comprising sensitisers of the formula (2) exhibit significant increases in efficiency compared with the prior art.

1-21. (canceled)

22. A composition for up-conversion comprising at least one sensitizer, which is a polymer, oligomer, dendrimer or small molecule, and at least one fluorescent organic emitter which is not an organic metal complex, wherein the sensitizer contains one or more structural units selected from the following compounds having the general formulae (1) and (2) and the triplet level T₁(S) of the sensitizer is higher, than the triplet level of the emitter T₁(E) 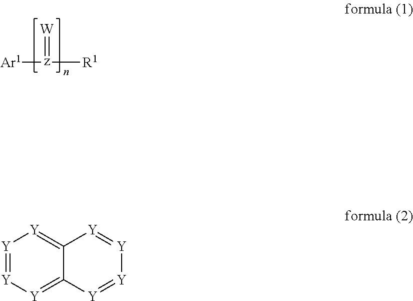
where the following applies to the symbols used:
n is either 1, 2 or 3;
W is, identically or differently on each occurrence, equal to O, S or Se;
Ar¹ is an aromatic or heteroaromatic ring or an aromatic or heteroaromatic ring system, where the rings is optionally substituted by one or more radicals R¹, with the proviso that Ar¹ contains at least 9 ring atoms;
Y is, identically or differently on each occurrence, equal to N or C(R¹), where at least 2 of the Y are equal to N;
Z is, identically or differently on each occurrence, C or S;
R¹ is, identically or differently on each occurrence, H, D, F, Cl, Br, I, N(R²)₂, CN, NO₂, Si(R²)₃, B(OR²)₂, C(═O)R², P(═O)(R²)₂, S(═O)R², S(═O)₂R², OSO₂R², a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which is optionally substituted by one or more radicals R², where one or more non-adjacent CH₂ groups is optionally replaced by R²C═CR², C≡C, Si(R²)₂, Ge(R²)₂, Sn(R²)₂, C═O, C═S, C═Se, C═NR², P(═O)(R²), SO, SO₂, NR², O, S or CONR² and where one or more H atoms is optionally replaced by D, F, Cl, Br, I, CN or NO₂, or an aromatic or heteroaromatic ring system having 5 to 60 aromatic ring atoms, which may in each case be substituted by one or more radicals R², or an aryloxy, arylalkoxy or heteroaryloxy group having 5 to 60 aromatic ring atoms, which is optionally substituted by one or more radicals R², or a diarylamino group, diheteroarylamino group or arylheteroarylamino group having 10 to 40 aromatic ring atoms, which is optionally substituted by one or more radicals R², or a combination of two or more of these groups or a crosslinkable group Q;
R² is, identically or differently on each occurrence, H, D, F, Cl, Br, I, N(R³)₂, CN, NO₂, Si(R³)₃, B(OR³)₂, C(═O)R³, P(═O)(R³)₂, S(═O)R³, S(═O)₂R³, OSO₂R³, a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which is optionally substituted by one or more radicals R³, where one or more non-adjacent CH₂ groups is optionally replaced by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³ and where one or more H atoms is optionally replaced by D, F, Cl, Br, I, CN or NO₂, or an aromatic or heteroaromatic ring system having 5 to 60 aromatic ring atoms, which may in each case be substituted by one or more radicals R³, or an aryloxy, arylalkoxy or heteroaryloxy group having 5 to 60 aromatic ring atoms, which is optionally substituted by one or more radicals R³, or a diarylamino group, diheteroarylamino group or arylheteroarylamino group having 10 to 40 aromatic ring atoms, which is optionally substituted by one or more radicals R³, or a combination of two or more of these groups; two or more adjacent radicals R² here may form a mono- or polycyclic, aliphatic or aromatic ring system with one another;
R³ is, identically or differently on each occurrence, H, D, F or an aliphatic, aromatic and/or heteroaromatic hydrocarbon radical having 1 to 20 C atoms, in which, in addition, one or more H atoms is optionally replaced by F; two or more substituents R³ here may also form a mono- or polycyclic, aliphatic or aromatic ring system with one another.

23. The composition according to claim 22, wherein the structural units of the formula (1) are selected from those of the formulae (3) to (7) 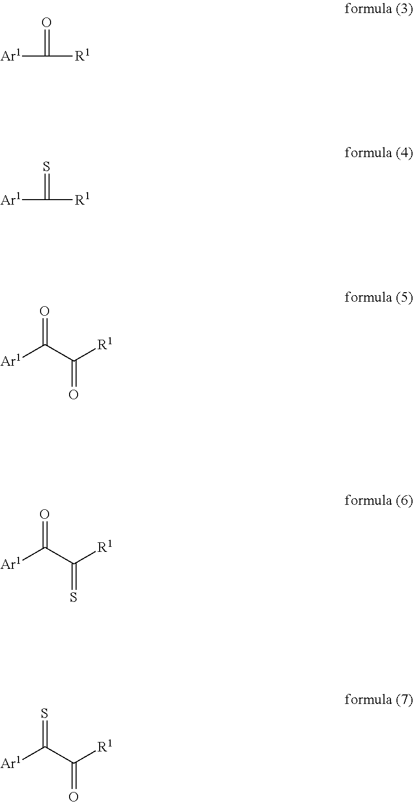
where the definitions of claim 22 apply to the symbols used.

24. The composition according to claim 22, wherein Ar¹ is selected from the groups of the formulae (8) to (14) 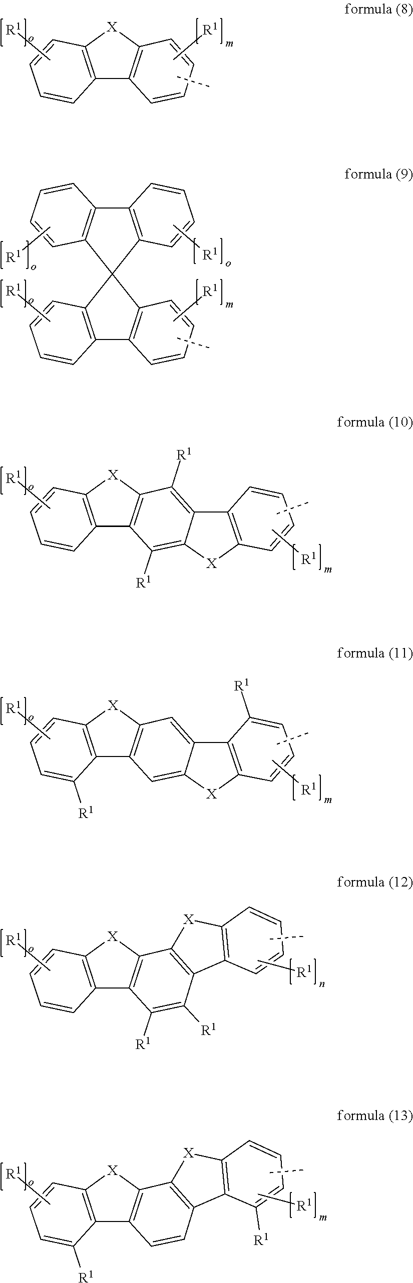 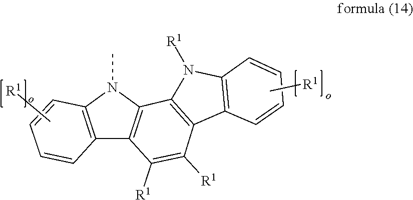
where R¹ has the same meaning described in claim 22, the dashed bond represents the linking position and furthermore:
X is, identically or differently on each occurrence, a divalent bridge selected from B(R¹), C(R¹)₂, Si(R¹)₂, C═O, C═NR¹, C═C(R¹)₂, O, S, S═O, SO₂, N(R¹), P(R¹) and P(═O)R¹;
m is on each occurrence, identically or differently, 0, 1, 2 or 3;
o is on each occurrence, identically or differently, 0, 1, 2, 3 or 4.

25. The composition according to claim 22, wherein the structural units of the formula (1) are selected from those of the formula (24) 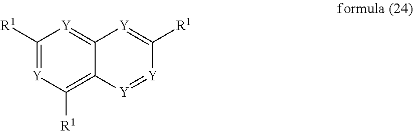
where the symbols are defined as in claim 22.

26. The composition according to claim 22, wherein the structural units of the formula (1) are selected from those of the formula (25) 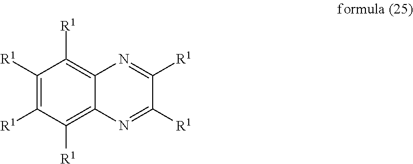
where R¹ is defined as in claim 22.

27. The composition according to claim 24, wherein R¹ is selected from formula (8) to (23) and one of the following formulae (34) to (222), where the compounds having the formulae (34) to (222) indicated is optionally substituted by one or more, identical or different radicals R²,
R¹ is, identically or differently on each occurrence, H, D, F, Cl, Br, I, N(R³)₂, CN, NO₂, Si(R³)₃, B(OR³)₂, C(═O)R³, P(═O)(R³)₂, S(═O)R³, S(═O)₂R³, OSO₂R³, a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which is optionally substituted by one or more radicals R³, where one or more non-adjacent CH₂ groups is optionally replaced by R³C═CR³, Si(R³)₂, Ge(R³)₂, Sn(R³)₂, C═O, C═S, C═Se, C═NR³, P(═O)(R³), SO, SO₂, NR³, O, S or CONR³ and where one or more 14 atoms is optionally replaced by D, F, Cl, Br, I, CN or NO₂, or an aromatic or heteroaromatic ring system having 5 to 60 aromatic ring atoms, which may in each case be substituted by one or more radicals R³, or an aryloxy, arylalkoxy or heteroaryloxy group having 5 to 60 aromatic ring atoms, which is optionally substituted by one or more radicals R³, or a diarylamino group, diheteroarylamino group or arylheteroarylamino group having 10 to 40 aromatic ring atoms, which is optionally substituted by one or more radicals R³, or a combination of two or more of these groups; two or more adjacent radicals R² here may form a mono- or polycyclic, aliphatic or aromatic ring system with one another;
R³ is, identically or differently on each occurrence, H, D, F or an aliphatic, aromatic and/or heteroaromatic hydrocarbon radical having 1 to 20 C atoms, in which, in addition, one or more H atoms is optionally replaced by F; two or more substituents R³ here may also form a mono- or polycyclic, aliphatic or aromatic ring system with one another;

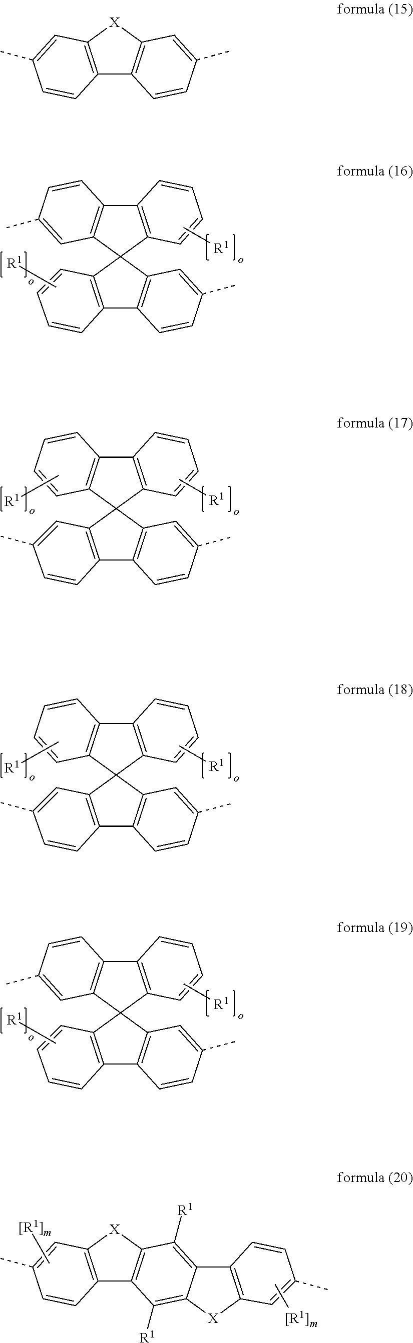

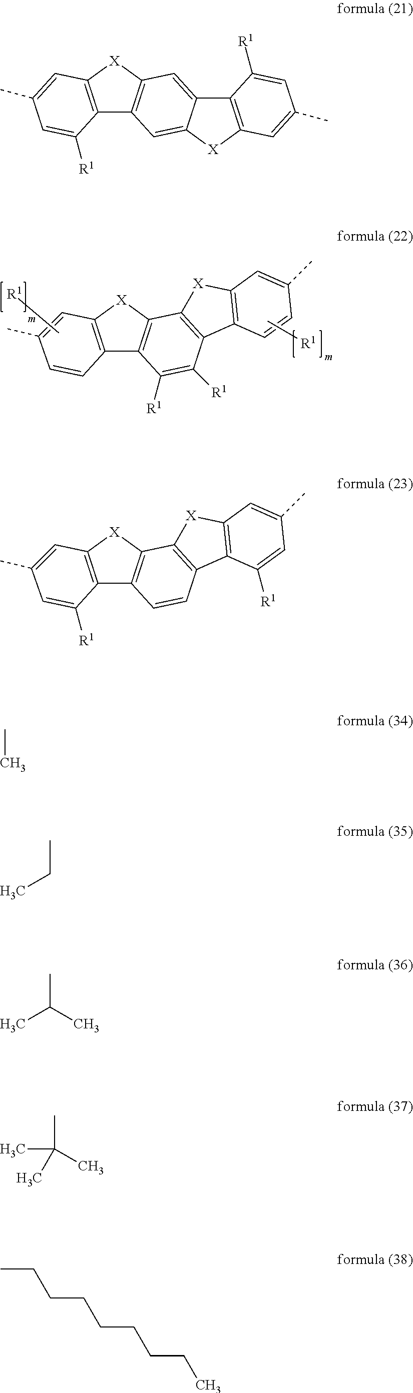

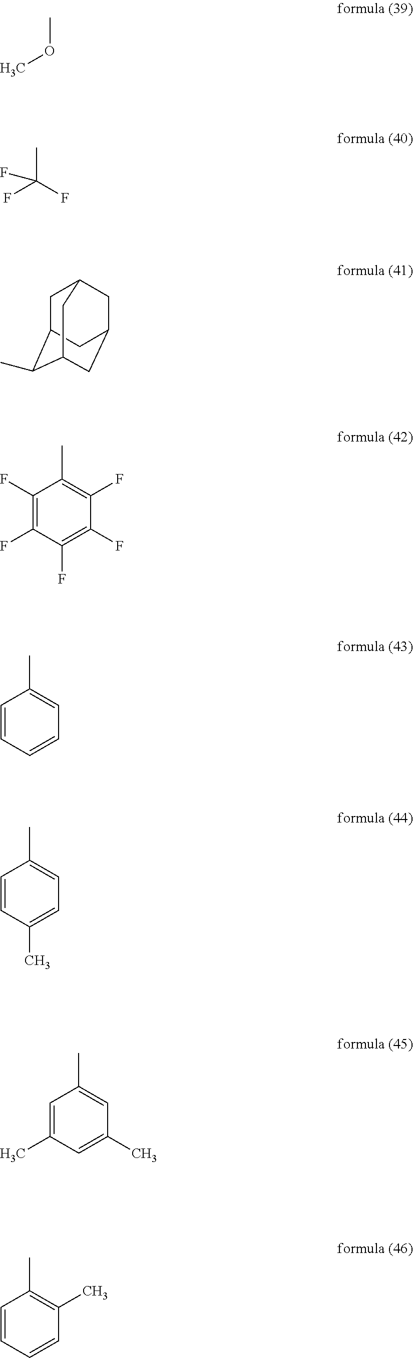

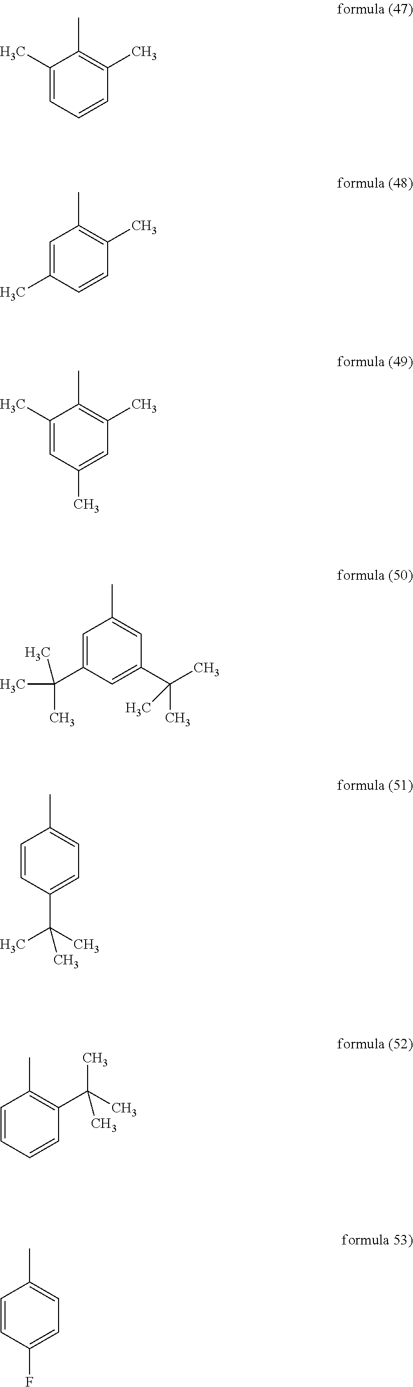

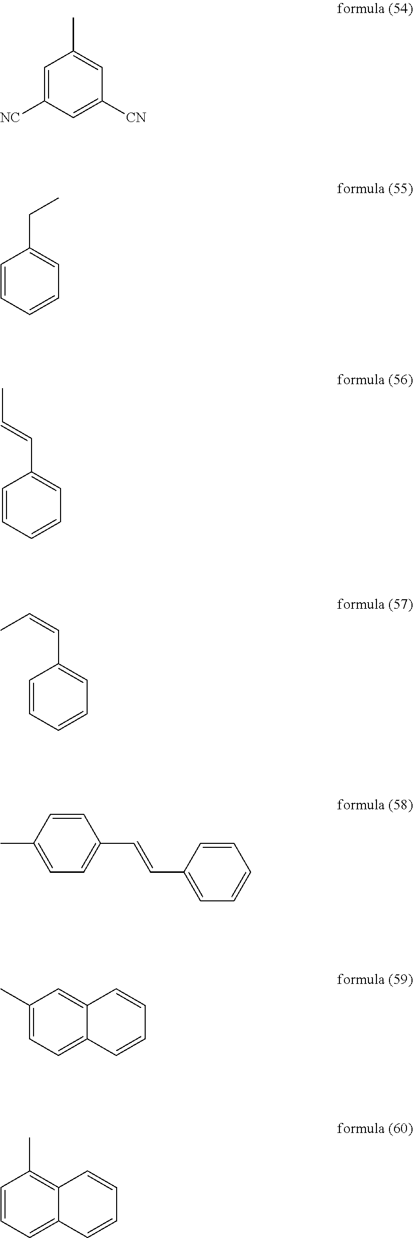

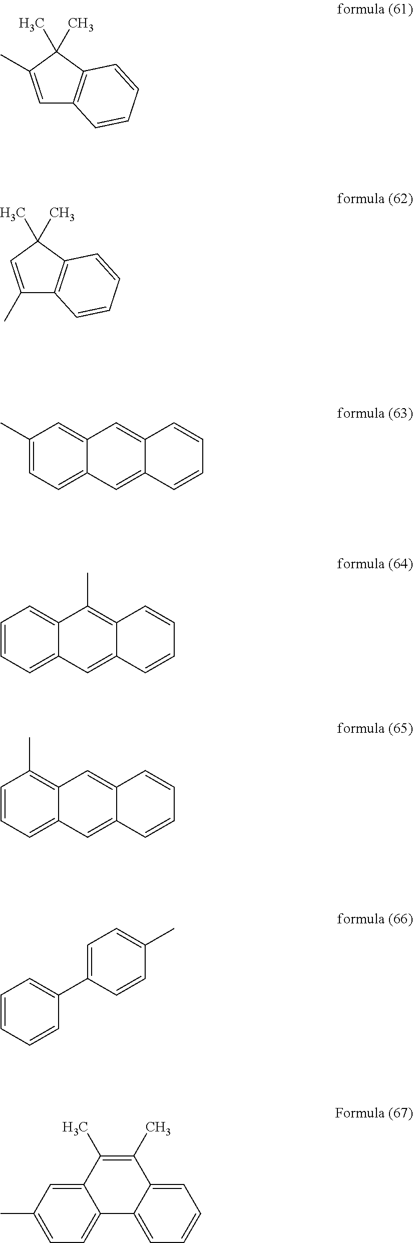

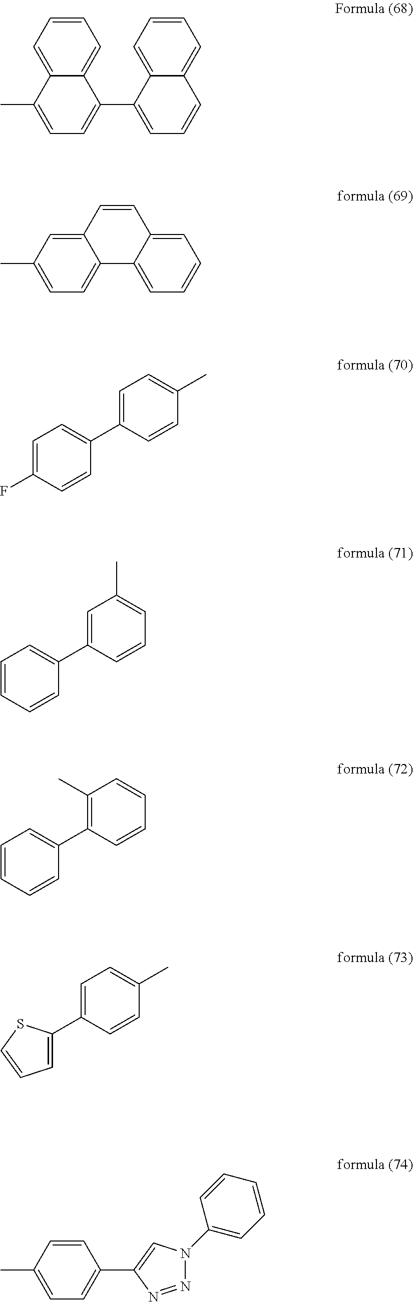

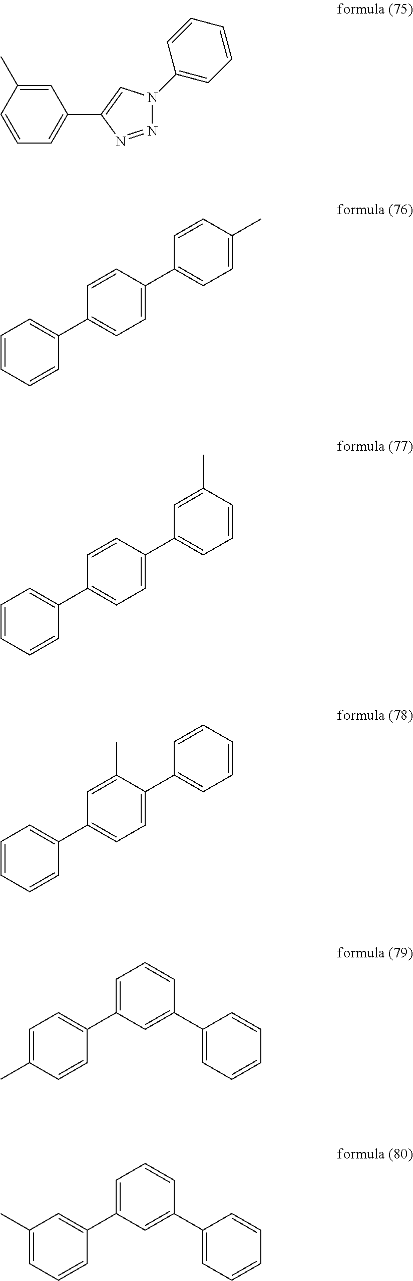

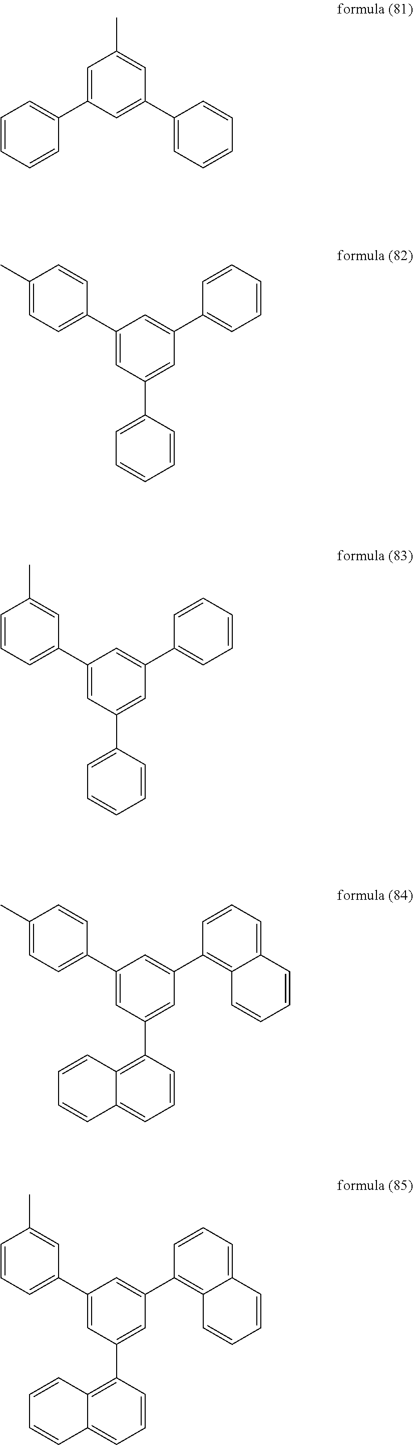

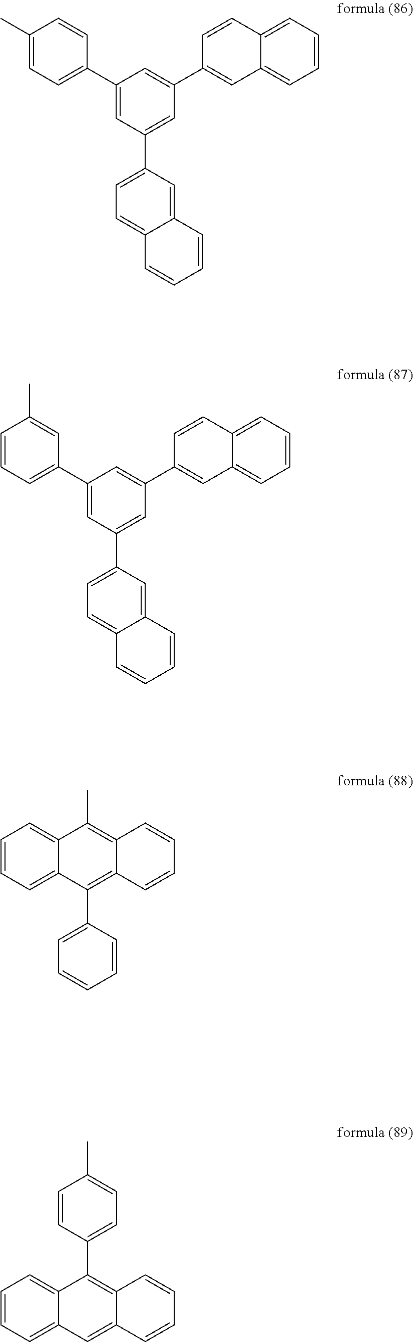

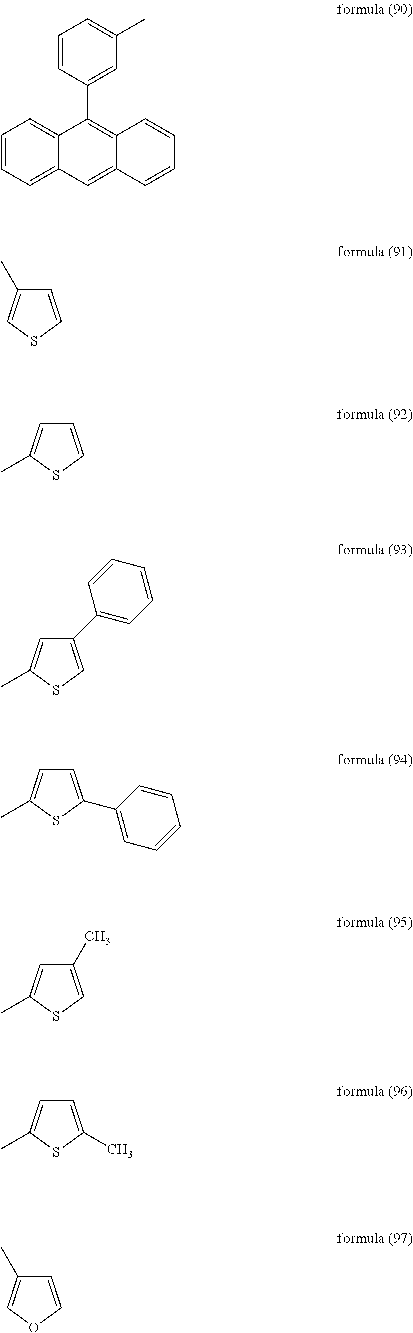

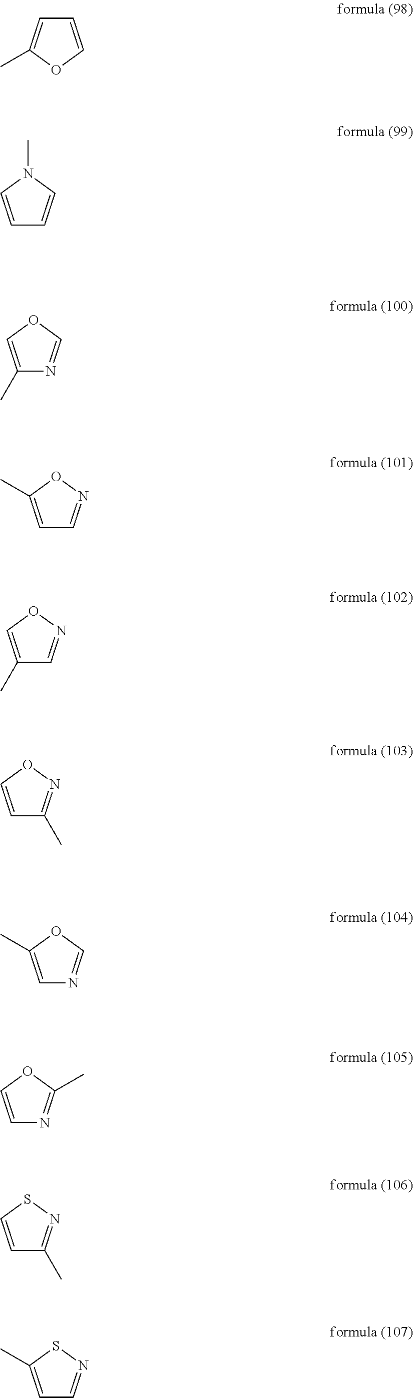

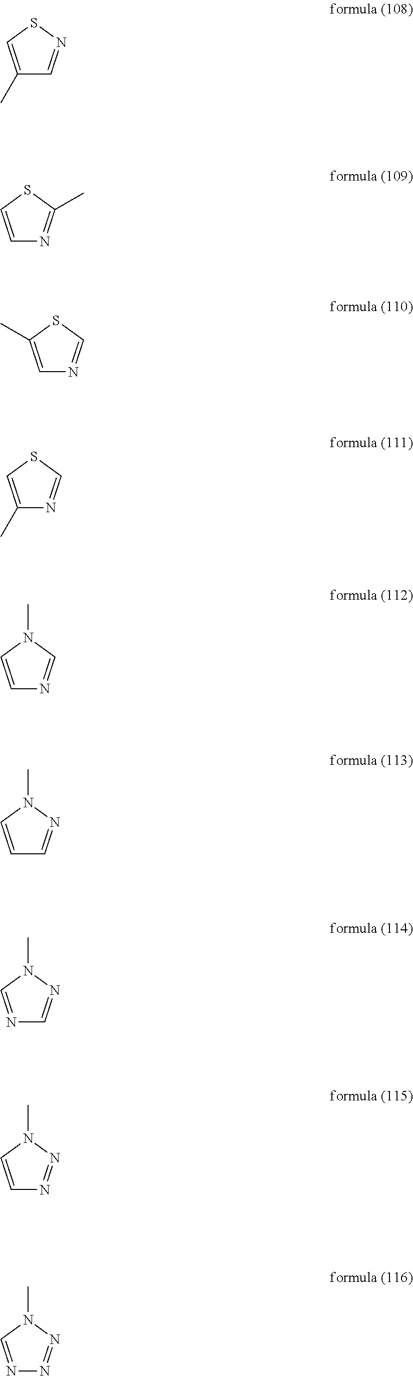

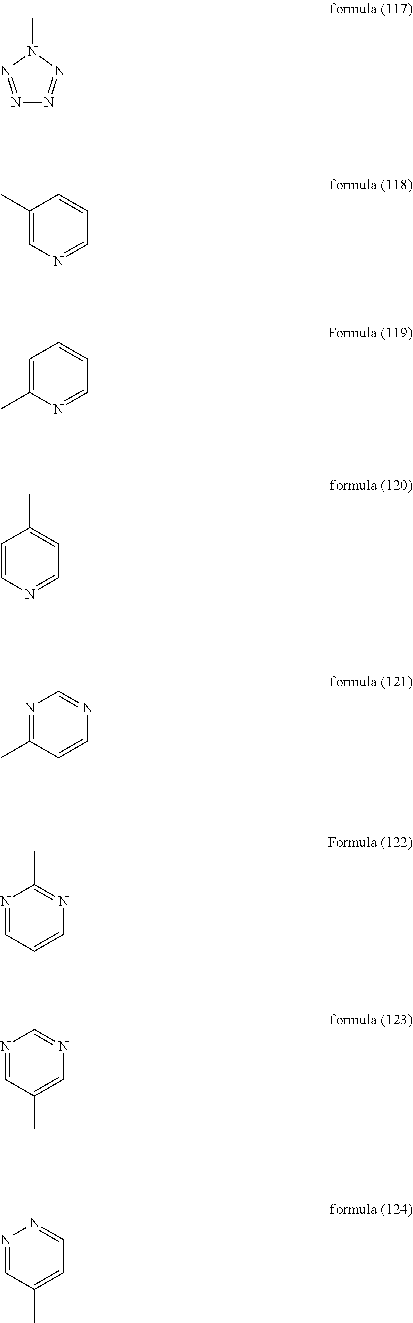

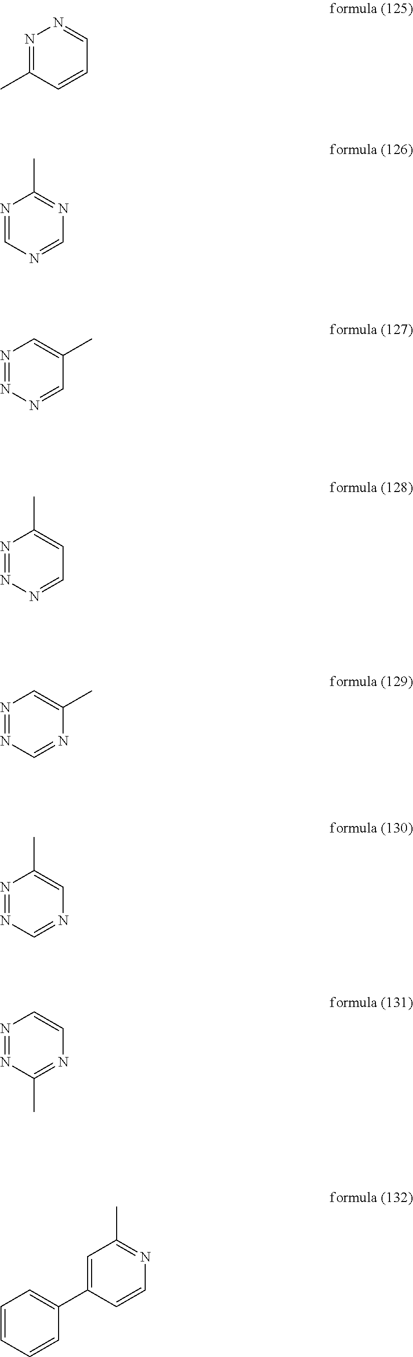

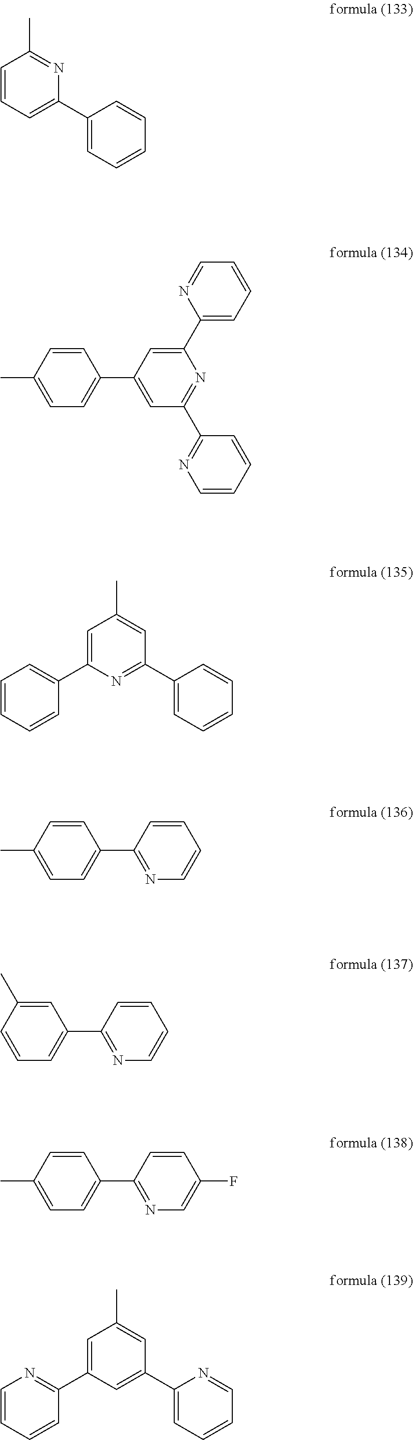

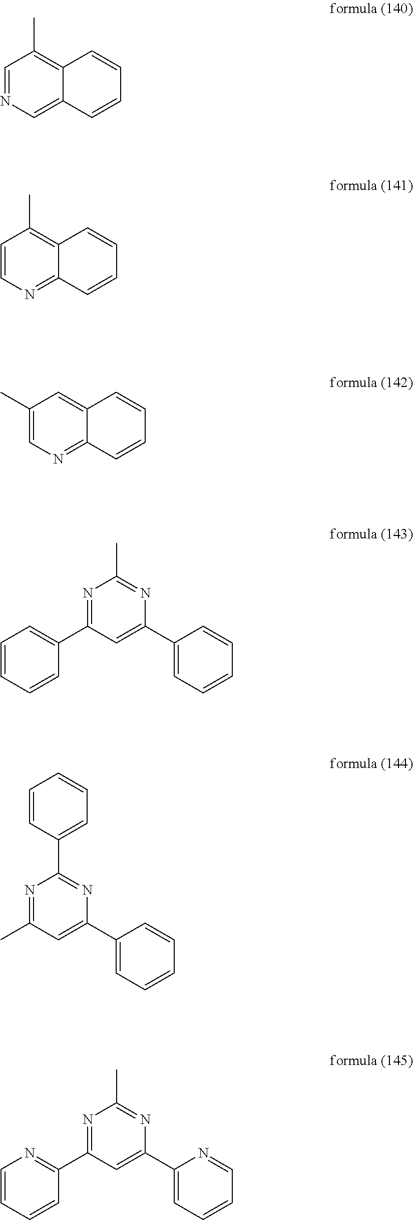 [SMILES: Cc1cncc2ccccc12 ; Cc1ccnc2ccccc12 ; Cc1cnc2ccccc2c1 ; Cc1nc(-c2ccccc2)cc(-c2ccccc2)n1 ; Cc1cc(-c2ccccc2)nc(-c2ccccc2)n1 ; Cc1nc(-c2ccccn2)cc(-c2ccccn2)n1]

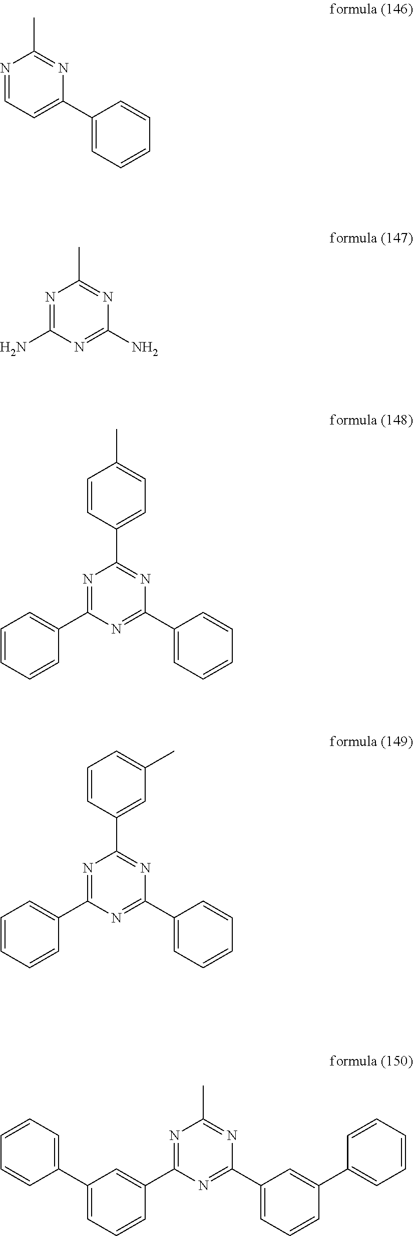

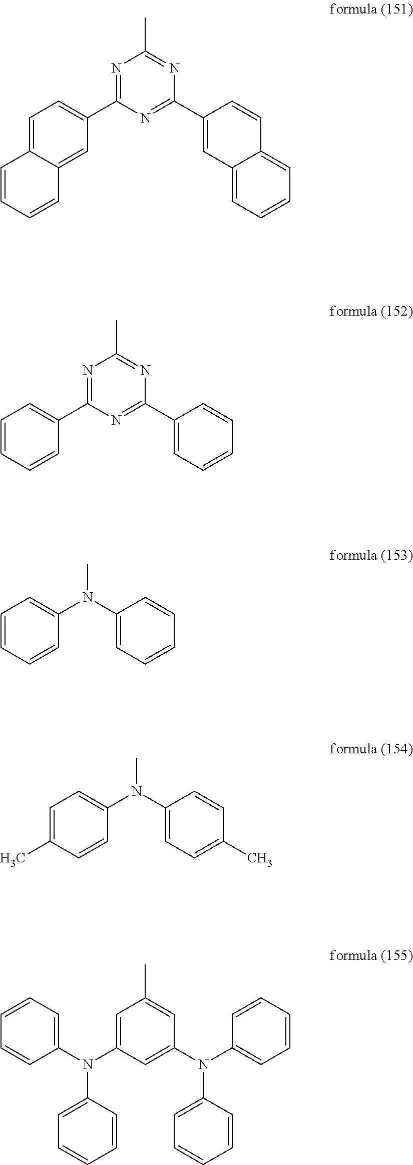

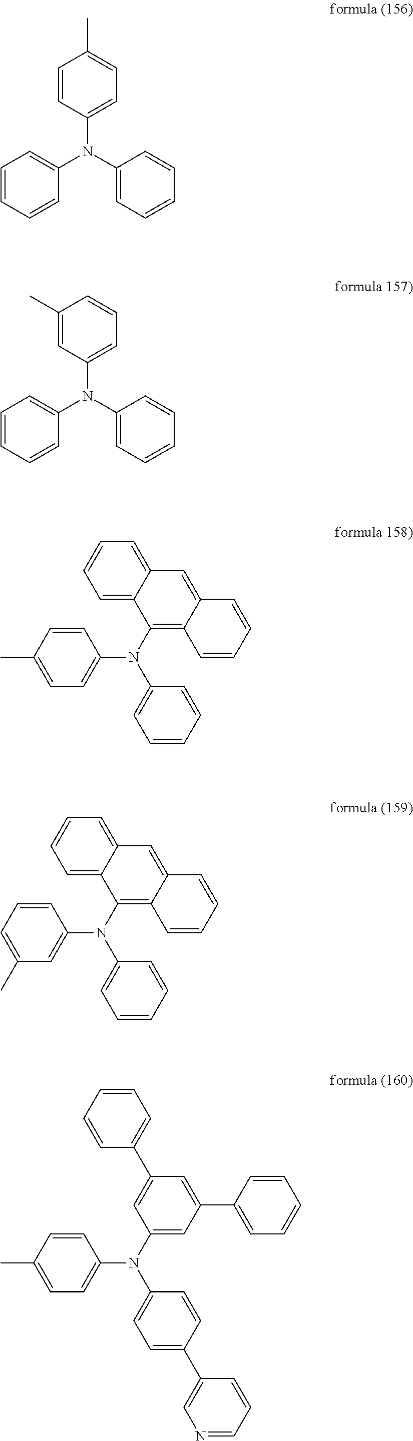 [SMILES: Cc1ccc(N(c2ccccc2)c2ccccc2)cc1 ; Cc1cccc(N(c2ccccc2)c2ccccc2)c1 ; Cc1ccc(N(c2ccccc2)c2c3ccccc3cc3ccccc23)cc1 ; Cc1cccc(N(c2ccccc2)c2c3ccccc3cc3ccccc23)c1 ; Cc1ccc(N(c2ccc(-c3cccnc3)cc2)c2cc(-c3ccccc3)cc(-c3ccccc3)c2)cc1]

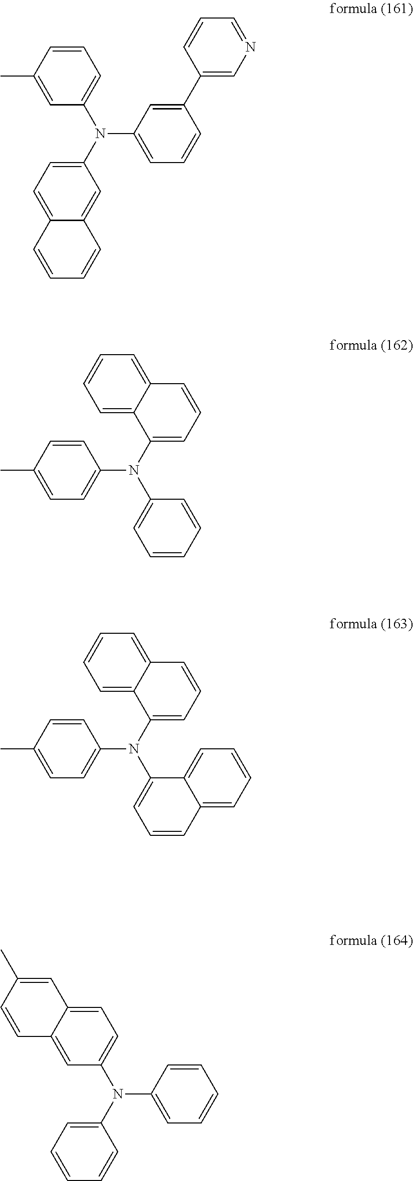 [SMILES: Cc1cccc(N(c2cccc(-c3cccnc3)c2)c2ccc3ccccc3c2)c1 ; Cc1ccc(N(c2ccccc2)c2cccc3ccccc23)cc1 ; Cc1ccc(N(c2cccc3ccccc23)c2cccc3ccccc23)cc1 ; Cc1ccc2cc(N(c3ccccc3)c3ccccc3)ccc2c1]

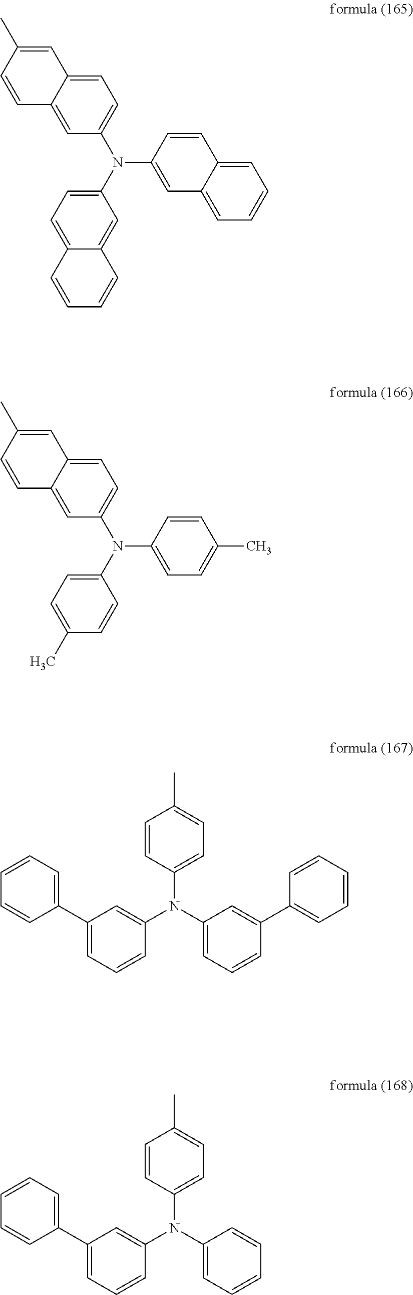 [SMILES: Cc1ccc2cc(N(c3ccc4ccccc4c3)c3ccc4ccccc4c3)ccc2c1 ; Cc1ccc(N(c2ccc(C)cc2)c2ccc3cc(C)ccc3c2)cc1 ; Cc1ccc(N(c2cccc(-c3ccccc3)c2)c2cccc(-c3ccccc3)c2)cc1 ; Cc1ccc(N(c2ccccc2)c2cccc(-c3ccccc3)c2)cc1]

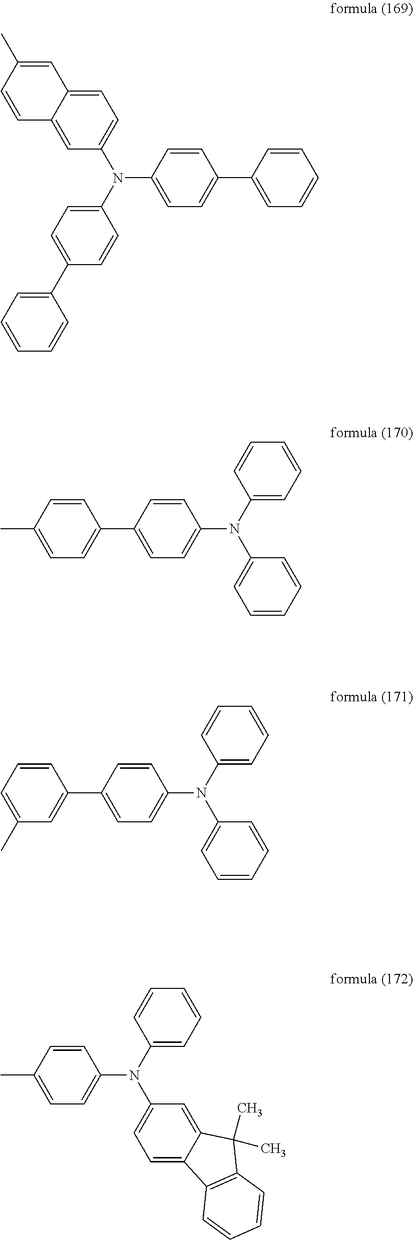

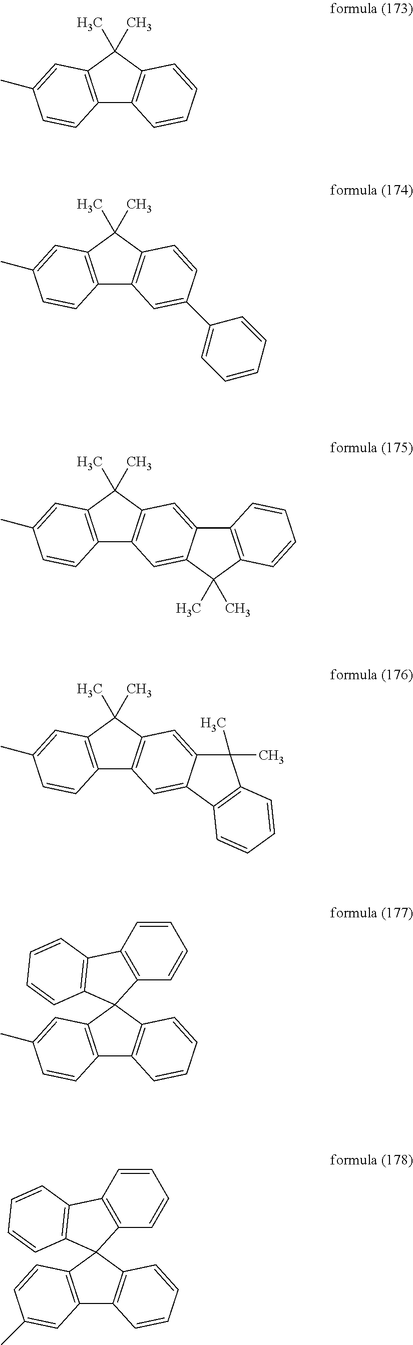

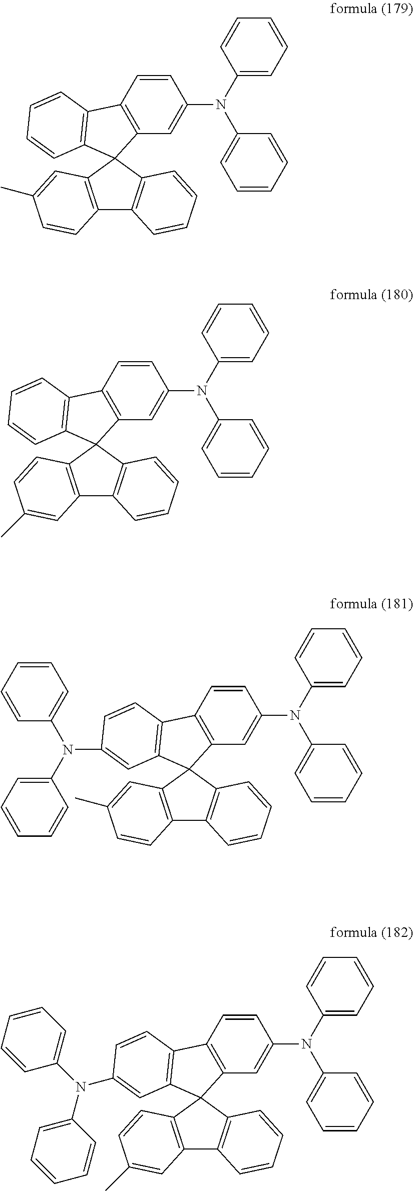 [SMILES: Cc1ccc2c(c1)C1(c3ccccc3-2)c2ccccc2-c2ccc(N(c3ccccc3)c3ccccc3)cc21 ; Cc1ccc2c(c1)-c1ccccc1C21c2ccccc2-c2ccc(N(c3ccccc3)c3ccccc3)cc21 ; Cc1ccc2c(c1)C1(c3ccccc3-2)c2cc(N(c3ccccc3)c3ccccc3)ccc2-c2ccc(N(c3ccccc3)c3ccccc3)cc21 ; Cc1ccc2c(c1)-c1ccccc1C21c2cc(N(c3ccccc3)c3ccccc3)ccc2-c2ccc(N(c3ccccc3)c3ccccc3)cc21]

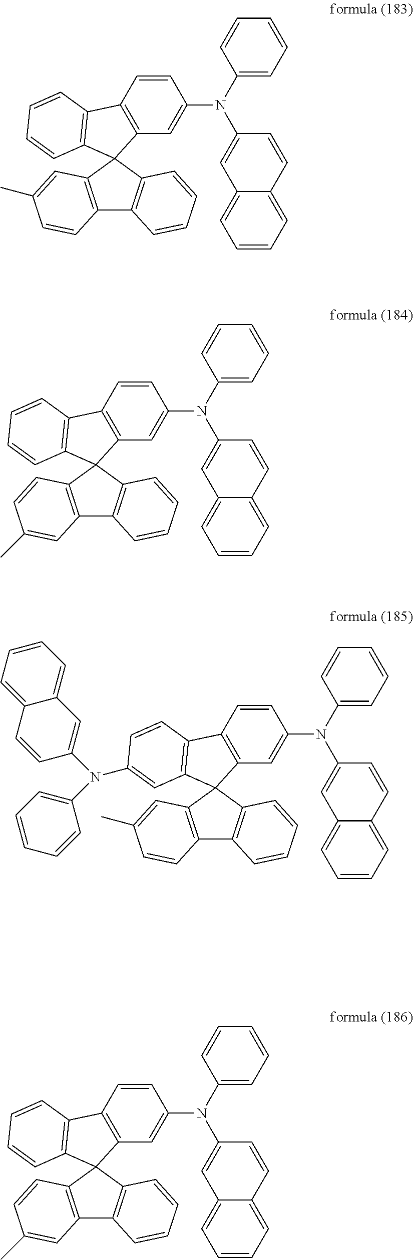 [SMILES: Cc1ccc2c(c1)C1(c3ccccc3-2)c2ccccc2-c2ccc(N(c3ccccc3)c3ccc4ccccc4c3)cc21 ; Cc1ccc2c(c1)-c1ccccc1C21c2ccccc2-c2ccc(N(c3ccccc3)c3ccc4ccccc4c3)cc21 ; Cc1ccc2c(c1)C1(c3ccccc3-2)c2cc(N(c3ccccc3)c3ccc4ccccc4c3)ccc2-c2ccc(N(c3ccccc3)c3ccc4ccccc4c3)cc21 ; Cc1ccc2c(c1)-c1ccccc1C21c2ccccc2-c2ccc(N(c3ccccc3)c3ccc4ccccc4c3)cc21]

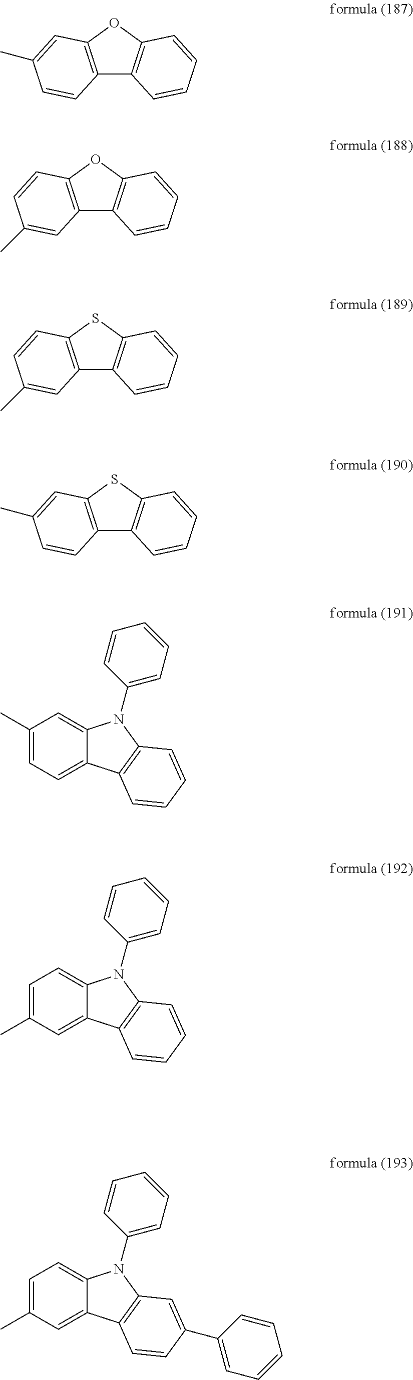

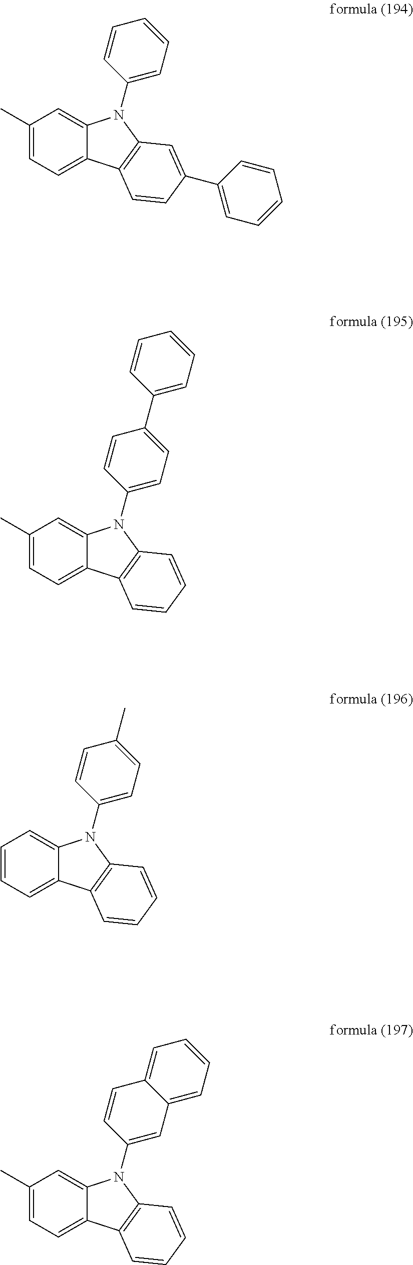 [SMILES: Cc1ccc2c3ccc(-c4ccccc4)cc3n(-c3ccccc3)c2c1 ; Cc1ccc2c3ccccc3n(-c3ccc(-c4ccccc4)cc3)c2c1 ; Cc1ccc(-n2c3ccccc3c3ccccc32)cc1 ; Cc1ccc2c3ccccc3n(-c3ccc4ccccc4c3)c2c1]

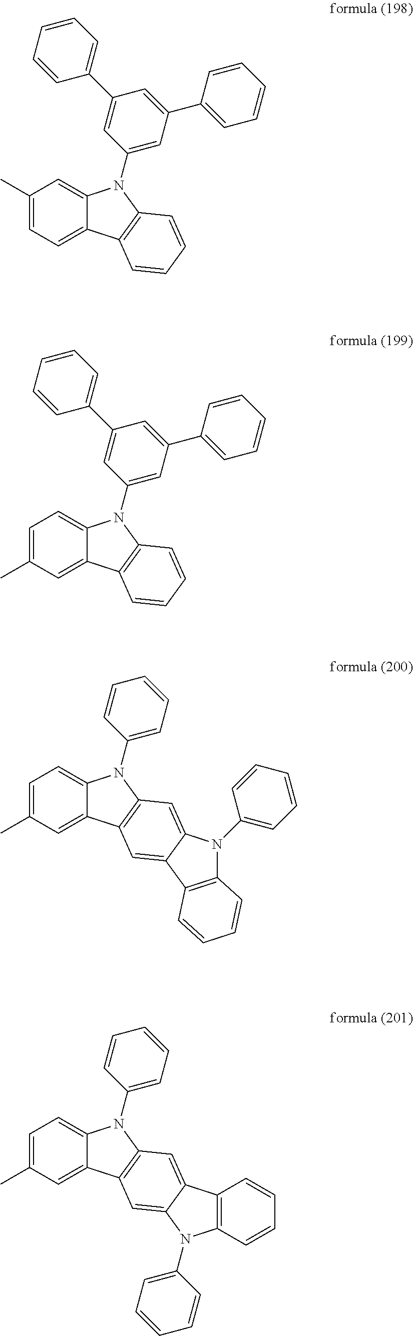 [SMILES: Cc1ccc2c3ccccc3n(-c3cc(-c4ccccc4)cc(-c4ccccc4)c3)c2c1 ; Cc1ccc2c(c1)c1ccccc1n2-c1cc(-c2ccccc2)cc(-c2ccccc2)c1 ; Cc1ccc2c(c1)c1cc3c4ccccc4n(-c4ccccc4)c3cc1n2-c1ccccc1 ; Cc1ccc2c(c1)c1cc3c(cc1n2-c1ccccc1)c1ccccc1n3-c1ccccc1]

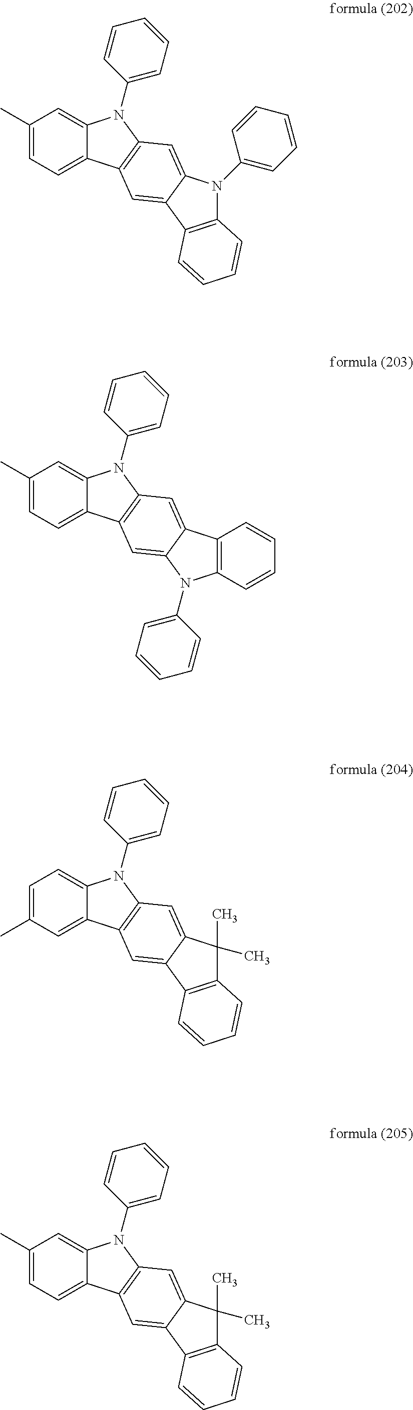

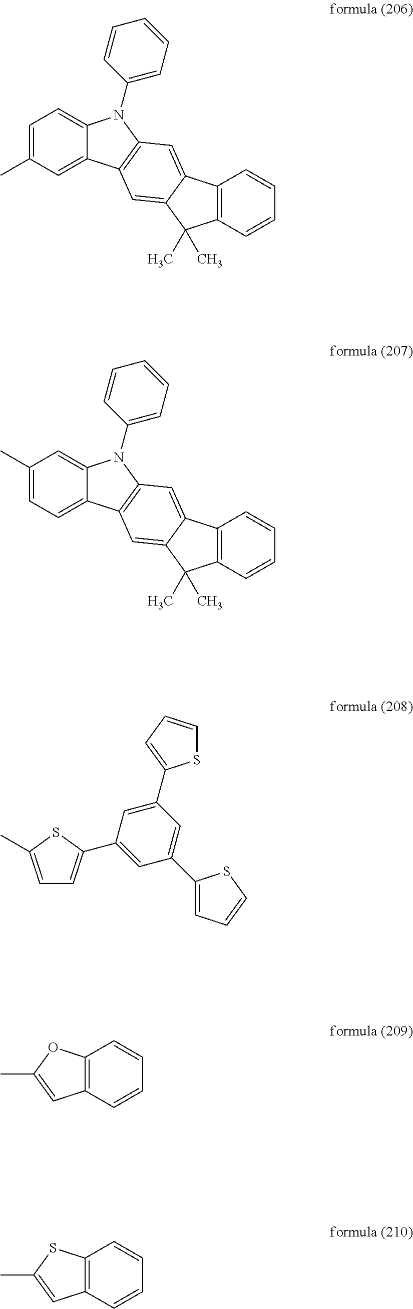

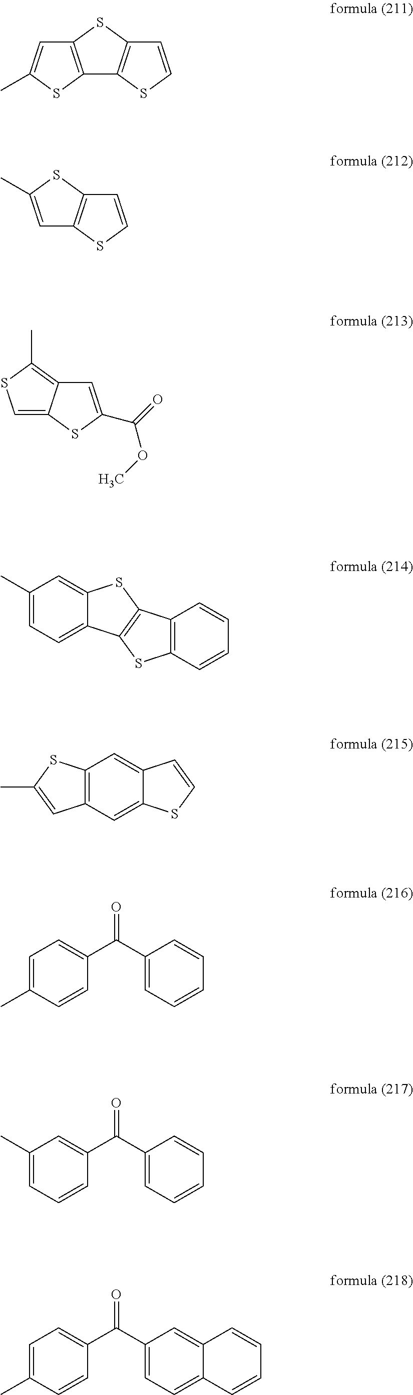

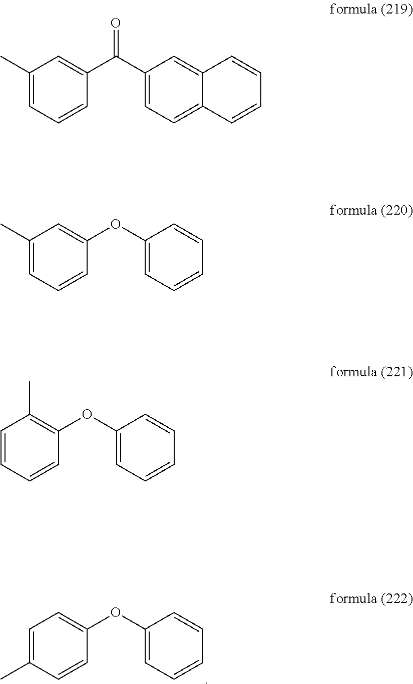 [SMILES: Cc1cccc(C(=O)c2ccc3ccccc3c2)c1 ; Cc1cccc(Oc2ccccc2)c1 ; Cc1ccccc1Oc1ccccc1 ; Cc1ccc(Oc2ccccc2)cc1]

28. The composition according to claim 22, wherein the first excited singlet level of the emitter S₁(E) is lower than that of the sensitizer S₁(S).

29. A process for up-conversion which comprises utilizing the composition according to claim 22.

30. The process according to claim 29, wherein blue light or radiation in UV region is generated.

31. The process according claim 29 wherein the up-conversion is in electroluminescent devices.

32. Use of a composition for up-conversion in electroluminescent devices for the generation of light or radiation in the UV region.

33. An electroluminescent device comprising one or more compositions for up-conversion for the generation of light or radiation in the UV region.

34. An optical and/or electronic device comprising at least one composition according to claim 22.

35. The device according to claim 34, wherein the device is an organic light-emitting diodes (OLED), organic light-emitting transistors, an organic light-emitting electrochemical cells (OLEC, LEC or LEEC), an organic light-emitting electrochemical transistors or an organic laser.

36. The device according to claim 34, wherein the device is for use in medical phototherapy.

37. The device according to claim 36 wherein the device is for use for the treatment of the skin by means of phototherapy.

38. The device according to claim 36, wherein psoriasis, vitiligo, jaundice of the newborn, dermatitis and atopic dermatitis, skin cancer, changes in the connective tissue is treated.

39. The device according to claim 36, wherein the treatment is carried out with a wavelength less than 400 nm.

40. A process for the cosmetic irradiation of the skin by means of phototherapy which comprises the device according to claim 34.

41. The process according to claim 40, wherein the cosmetic application is an application in the area of acne, cellulite, skin reddening, skin wrinkling or skin rejuvenation.

42. A method for the treatment of the skin which comprises treating the skin by means of phototherapy through the use of a luminescent comprising the composition according to claim 22.
